SEQUENCE LISTING (22 sequences)

SEQ ID NO: 1 - DNA, 1938 bp, Homo sapiens
  source 1..1938: mol_type = genomic DNA
  CDS 153..1742
cagaggccgcgcctgctgctgagcagatgcagtagccgaaactgcgcggaggcacagaggccggggagagcgttctgggtccgagggtccaggtaggggttgagccaccatctgaccgcaagctgcgtcgtgtcgccggttctgcaggcaccatgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgagcgccgcgttgtcgcgagctaccggcgcagaagtggtggcacacgggcgccctctaccgcatcggcgaccttcaggccttccagggccacggcgcgggcaacctggcgggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgagaactcgtggttctccactcaggttgacactgtggccaccaaggtgaaggatgctctggagttttggctgcaagctggcgtggatgggttccaggttcgggacatagagaatctgaaggatgcatcctcattcttggctgagtggcaaaatatcaccaagggcttcagtgaagacaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagtttgtctcaggcaaggctcctgacttccttcttgccggctcaacttctccgactctaccagctgatgctcttcaccctgccagggacccctgttttcagctacggggatgagattggcctggatgcagctgcccttcctggacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctgacttcagcctgacatggacccactacccttctcctttccttcccaggccctttggcttctgatttttctcttttttaaaaacaaacaaacaaactgttgcagattatgagtgaacccccaaatagggtgttttctgccttcaaataaaagtcacccctgcatggtgaagtcttccctctgcttctctcataaaaaaa

SEQ ID NO: 2 - protein, 529 aa, Homo sapiens
MSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 3 - DNA, 4543 bp, Homo sapiens
  source 1..4543: mol_type = genomic DNA
  CDS 73..1596
cggcgggcggcgcgcacactgctcgctgggccgcggctcccgggtgtcccaggcccggccggtgcgcagagcatggcgggtgcgggcccgaagcggcgcgcgctagcggcgccggcggccgaggagaaggaagaggcgcgggagaagatgctggccgccaagagcgcggacggctcggcgccggcaggcgagggcgagggcgtgaccctgcagcggaacatcacgctgctcaacggcgtggccatcatcgtggggaccattatcggctcgggcatcttcgtgacgcccacgggcgtgctcaaggaggcaggctcgccggggctggcgctggtggtgtgggccgcgtgcggcgtcttctccatcgtgggcgcgctctgctacgcggagctcggcaccaccatctccaaatcgggcggcgactacgcctacatgctggaggtctacggctcgctgcccgccttcctcaagctctggatcgagctgctcatcatccggccttcatcgcagtacatcgtggccctggtcttcgccacctacctgctcaagccgctcttccccacctgcccggtgcccgaggaggcagccaagctcgtggcctgcctctgcgtgctgctgctcacggccgtgaactgctacagcgtgaaggccgccacccgggtccaggatgcctttgccgccgccaagctcctggccctggccctgatcatcctgctgggcttcgtccagatcgggaagggtgatgtgtccaatctagatcccaacttctcatttgaaggcaccaaactggatgtggggaacattgtgctggcattatacagcggcctctttgcctatggaggatggaattacttgaatttcgtcacagaggaaatgatcaacccctacagaaacctgcccctggccatcatcatctccctgcccatcgtgacgctggtgtacgtgctgaccaacctggcctacttcaccaccctgtccaccgagcagatgctgtcgtccgaggccgtggccgtggacttcgggaactatcacctgggcgtcatgtcctggatcatccccgtcttcgtgggcctgtcctgcttcggctccgtcaatgggtccctgttcacatcctccaggctcttcttcgtggggtcccgggaaggccacctgccctccatcctctccatgatccacccacagctcctcacccccgtgccgtccctcgtgttcacgtgtgtgatgacgctgctctacgccttctccaaggacatcttctccgtcatcaacttcttcagcttcttcaactggctctgcgtggccctggccatcatcggcatgatctggctgcgccacagaaagcctgagcttgagcggcccatcaaggtgaacctggccctgcctgtgttcttcatcctggcctgcctcttcctgatcgccgtctccttctggaagacacccgtggagtgtggcatcggcttcaccatcatcctcagcgggctgcccgtctacttcttcggggtctggtggaaaaacaagcccaagtggctcctccagggcatcttctccacgaccgtcctgtgtcagaagctcatgcaggtggtcccccaggagacatagccaggaggccgagtggctgccggaggagcatgcgcagaggccagttaaagtagatcacctcctcgaacccactccggttccccgcaacccacagctcagctgcccatcccagtccctcgccgtccctcccaggtcgggcagtggaggctgctgtgaaaactctggtacgaatctcatccctcaactgagggccagggacccaggtgtgcctgtgctcctgcccaggagcagcttttggtctccttgggccctttttcccttccctcctttgtttacttatatatatattttttttaaacttaaattttgggtcaacttgacaccactaagatgattttttaaggagctgggggaaggcaggagccttcctttctcctgccccaagggcccagaccctgggcaaacagagctactgagacttggaacctcattgctaccacagacttgcactgaagccggacagctgcccagacacatgggcttgtgacattcgtgaaaaccaaccctgtgggcttatgtctctgccttagggtttgcagagtggaaactcagccgtagggtggcactgggagggggtgggggatctgggcaaggtgggtgattcctcccaggaggtgcttgaggccccgatggactcctgaccataatcctagccccgagacaccatcctgagccagggaacagccccagggttggggggtgccggcatctcccctagctcaccaggcctggcctctgggcagtgtggcctcttggctatttctgtgtccagttttggaggctgagttctggttcatgcagacaaagccctgtccttcagtcttctagaaacagagacaagaaaggcagacacaccgcggccaggcacccatgtgggcgcccaccctgggctccacacagcagtgtcccctgccccagaggtcgcagctaccctcagcctccaatgcattggcctctgtaccgcccggcagccccttctggccggtgctgggttcccactcccggcctaggcacctccccgctctccctgtcacgctcatgtcctgtcctggtcctgatgcccgttgtctaggagacagagccaagcactgctcacgtctctgccgcctgcgtttggaggcccctgggctctcacccagtccccacccgcctgcagagagggaactagggcaccccttgtttctgttgttcccgtgaatttttttcgctatgggaggcagccgaggcctggccaatgcggcccactttcctgagctgtcgctgcctccatggcagcagccagggacccccagaacaagaagaccccgcaggatccctcctgagctcggggggctctgccttctcaggccccgggcttcccttctccccagccagaggtggagccaagtggtccagcgtcactccagtgctcagctgtggctggaggagctggcctgtggcacagccctgagtgtcccaagccgggagccaacgaagccggacacggcttcactgaccagcggctgctcaagccgcaagctctcagcaagtgcccagtggagcctgccgcccccgcctgggcaccgggaccccctcaccatccagtgggcccggagaaacctgatgaacagtttggggactcaggaccagatgtccgtctctcttgcttgaggaatgaagacctttattcacccctgccccgttgcttcccgctgcacatggacagacttcacagcgtctgctcataggacctgcatccttcctggggacgaattccactcgtccaagggacagcccacggtctggaggccgaggaccaccagcaggcaggtggactgactgtgttgggcaagacctcttccctctgggcctgttctcttggctgcaaataaggacagcagctggtgccccacctgcctggtgcattgctgtgtgaatccaggaggcagtggacatcgtaggcagccacggccccgggtccaggagaagtgctccctggaggcacgcaccactgcttcccactggggccggcggggcccacgcacgacgtcagcctcttaccttcccgcctcggctaggggtcctcgggatgccgttctgttccaacctcctgctctgggacgtggacatgcctcaaggatacagggagccggcggcctctcgacggcacgcacttgcctgttggctgctgcggctgtgggcgagcatgggggctgccagcgtctgttgtggaaagtagctgctagtgaaatggctggggccgctggggtccgtcttcacactgcgcaggtctcttctgggcgtctgagctggggtgggagctcctccgcagaaggttggtggggggtccagtctgtgatccttggtgctgtgtgccccactccagcctggggaccccacttcagaaggtaggggccgtgtcccgcggtgctgactgaggcctgcttccccctccccctcctgctgtgctggaattccacagggaccagggccaccgcaggggactgtctcagaagacttgatttttccgtccctttttctccacactccactgacaaacgtccccagcggtttccacttgtgggcttcaggtgttttcaagcacaacccaccacaacaagcaagtgcattttcagtcgttgtgcttttttgttttgtgctaacgtcttactaatttaaagatgctgtcggcaccatgtttatttatttccagtggtcatgctcagccttgctgctctgcgtggcgcaggtgccatgcctgctccctgtctgtgtcccagccacgcagggccatccactgtgacgtcggccgaccaggctggacaccctctgccgagtaatgacgtgtgtggctgggaccttctttattctgtgttaatggctaacctgttacactgggctgggttgggtagggtgttctggcttttttgtggggtttttatttttaaagaaacactcaatcatccta

SEQ ID NO: 4 - protein, 507 aa, Homo sapiens
MAGAGPKRRALAAPAAEEKEEAREKMLAAKSADGSAPAGEGEGVTLQRNITLLNGVAIIVGTIIGSGIFVTPTGVLKEAGSPGLALVVWAACGVFSIVGALCYAELGTTISKSGGDYAYMLEVYGSLPAFLKLWIELLIIRPSSQYIVALVFATYLLKPLFPTCPVPEEAAKLVACLCVLLLTAVNCYSVKAATRVQDAFAAAKLLALALIILLGFVQIGKGDVSNLDPNFSFEGTKLDVGNIVLALYSGLFAYGGWNYLNFVTEEMINPYRNLPLAIIISLPIVTLVYVLTNLAYFTTLSTEQMLSSEAVAVDFGNYHLGVMSWIIPVFVGLSCFGSVNGSLFTSSRLFFVGSREGHLPSILSMIHPQLLTPVPSLVFTCVMTLLYAFSKDIFSVINFFSFFNWLCVALAIIGMIWLRHRKPELERPIKVNLALPVFFILACLFLIAVSFWKTPVECGIGFTIILSGLPVYFFGVWWKNKPKWLLQGIFSTTVLCQKLMQVVPQET

SEQ ID NO: 5 - DNA, 4884 bp, Homo sapiens
  source 1..4884: mol_type = genomic DNA
  CDS 697..2409
gcacgcggttctccctgatcccggagctgggctcagggctcggactcagtcctgcagcgcctctaggctgcggatccgcgcttcaaccacctgctttgcgctgcgtccggggaagtggggaggagacgggagggagggaggaggcggggagaggaggaaagaggcagcttacacacgccttccagtccctctactcagagcagcccggagaccgctgccgccgctgccgctgctaccaccgctgccacctgaggagacccgccgcccccccgtcgccgcctcctgcgagtccttcttagcacctggcgtttcatgcacattgccactgccattattattatcattccaatacaaggaaaataaaagaagataccagcgaaaagaaccgcttacacctttccgaattactcaagtgtctcctggaaacagagggtcgttgtccccggaggagcagccgaagggcccgtgggctggtgttgaccgggagggaggaggagttgggggcattgcgtggtggaaagttgcgtgcggcagagaaccgaaggtgcagcgccacagcccaggggacggtgtgtctgggagaagacgctgcccctgcgtcgggacccgccagcgcgcgggcaccgcggggcccgggacgacgccccctcctgcggcgtggactccgtcagtggcccaccaagaaggaggaggaatatggaatccaagggggccagttcctgccgtctgctcttctgcctcttgatctccgccaccgtcttcaggccaggccttggatggtatactgtaaattcagcatatggagataccattatcataccttgccgacttgacgtacctcagaatctcatgtttggcaaatggaaatatgaaaagcccgatggctccccagtatttattgccttcagatcctctacaaagaaaagtgtgcagtacgacgatgtaccagaatacaaagacagattgaacctctcagaaaactacactttgtctatcagtaatgcaaggatcagtgatgaaaagagatttgtgtgcatgctagtaactgaggacaacgtgtttgaggcacctacaatagtcaaggtgttcaagcaaccatctaaacctgaaattgtaagcaaagcactgtttctcgaaacagagcagctaaaaaagttgggtgactgcatttcagaagacagttatccagatggcaatatcacatggtacaggaatggaaaagtgctacatccccttgaaggagcggtggtcataatttttaaaaaggaaatggacccagtgactcagctctataccatgacttccaccctggagtacaagacaaccaaggctgacatacaaatgccattcacctgctcggtgacatattatggaccatctggccagaaaacaattcattctgaacaggcagtatttgatatttactatcctacagagcaggtgacaatacaagtgctgccaccaaaaaatgccatcaaagaaggggataacatcactcttaaatgcttagggaatggcaaccctcccccagaggaatttttgttttacttaccaggacagcccgaaggaataagaagctcaaatacttacacactgacggatgtgaggcgcaatgcaacaggagactacaagtgttccctgatagacaaaaaaagcatgattgcttcaacagccatcacagttcactatttggatttgtccttaaacccaagtggagaagtgactagacagattggtgatgccctacccgtgtcatgcacaatatctgctagcaggaatgcaactgtggtatggatgaaagataacatcaggcttcgatctagcccgtcattttctagtcttcattatcaggatgctggaaactatgtctgcgaaactgctctgcaggaggttgaaggactaaagaaaagagagtcattgactctcattgtagaaggcaaacctcaaataaaaatgacaaagaaaactgatcccagtggactatctaaaacaataatctgccatgtggaaggttttccaaagccagccattcaatggacaattactggcagtggaagcgtcataaaccaaacagaggaatctccttatattaatggcaggtattatagtaaaattatcatttcccctgaagagaatgttacattaacttgcacagcagaaaaccaactggagagaacagtaaactccttgaatgtctctgctaatgaaaacagagaaaaggtgaatgaccaggcaaaactaattgtgggaatcgttgttggtctcctccttgctgcccttgttgctggtgtcgtctactggctgtacatgaagaagtcaaagactgcatcaaaacatgtaaacaaggacctcggtaatatggaagaaaacaaaaagttagaagaaaacaatcacaaaactgaagcctaagagagaaactgtcctagttgtccagagataaaaatcatatagaccaattgaagcatgaacgtggattgtatttaagacataaacaaagacattgacagcaattcatggttcaagtattaagcagttcattctaccaagctgtcacaggttttcagagaattatctcaagtaaaacaaatgaaatttaattacaaacaataagaacaagttttggcagccatgataataggtcatatgttgtgtttggttcaattttttttccgtaaatgtctgcactgaggatttctttttggtttgccttttatgtaaattttttacgtagctatttttatacactgtaagctttgttctgggagttgctgttaatctgatgtataatgtaatgtttttatttcaattgtttatatggataatctgagcaggtacatttctgattctgattgctatcagcaatgccccaaactttctcataagcacctaaaacccaaaggtggcagcttgtgaagattggggacactcatattgccctaattaaaaactgtgatttttatcacaagggaggggaggccgagagtcagactgatagacaccataggagccgactctttgatatgccaccagcgaactctcagaaataaatcacagatgcatatagacacacatacataatggtactcccaaactgacaattttacctattctgaaaaagacataaaacagaatttggtagcacttacctctacagacacctgctaataaattattttctgtcaaaagaaaaaacacaagcatgtgtgagagacagtttggaaaaatcatggtcaacattcccattttcatagatcacaatgtaaatcactataattacaaattggtgttaaatcctttgggttatccactgccttaaaattatacctatttcatgtttaaaaagatatcaatcagaattggagtttttaacagtggtcattatcaaagctgtgttattttccacagaatatagaatatatatttttttcgtgtgtgtttttgttaactaccctacagatattgaatgcaccttgagataatttagtgtttttaactgatacataatttatcaagcagtacatgaaagtgtaataataaaatgtctatgtatctttagttacattcaaatttgtaactttataaacatgttttatgcttgaggaaatttttaaggtggtagtataaatggaaactttttgaagtagaccagatatgggctacttgtgactagacttttaaactttgctctttcaagcagaagcctggtttctgggagaacactgcacagcgatttctttcccaggatttacacaactttaaagggaagataaatgaacatcagatttctaggtatagaactatgttattgaaaggaaaaggaaaactggtgtttgtttcttagactcatgaaataaaaaattatgaaggcaatgaaaaataaattgaaaattaaagtcagatgagaataggaataatactttgccacttctgcattatttagaaacatacgttattgtacatttgtaaaccatttactgtctgggcaatagtgactccgtttaataaaagcttccgtagtgcattggtatggattaaatgcataaaatattcttagactcgatgctgtataaaatattatgggaaaaaaagaaaatacgttattttgcctctaaacttttattgaagttttatttggcaggaaaaaaaattgaatcttggtcaacatttaaaccaaagtaaaaggggaaaaaccaaagttatttgttttgcatggctaagccattctgttatctctgtaaatactgtgatttcttttttattttctctttagaattttgttaaagaaattctaaaatttttaaacacctgctctccacaataaatcacaaacactaaaataaaattacttccatataaatattattttctcttttggtgtgggagatcaaaggtttaaagtctaacttctaagatatatttgcagaaagaagcaacatgacaatagagagagttatgctacaattatttcttggtttccacttgcaatggttaattaagtccaaaaacagctgtcagaacctcgagagcagaacatgagaaactcagagctctggaccgaaagcagaaagtttgccgggaaaaaaaaagacaacattattaccatcgattcagtgcctggataaagaggaaagcttacttgtttaatggcagccacatgcacgaagatgctaagaagaaaaagaattccaaatcctcaacttttgaggtttcggctctccaatttaactctttggcaacaggaaacaggttttgcaagttcaaggttcactccctatatgtgattataggaattgtttgtggaaatggattaacatacccgtctatgcctaaaagataataaaactgaaatatgtcttcacaggtctcccacaaaaaaaaaaaaaa

SEQ ID NO: 6 - protein, 570 aa, Homo sapiens
MESKGASSCRLLFCLLISATVFRPGLGWYTVNSAYGDTIIIPCRLDVPQNLMFGKWKYEKPDGSPVFIAFRSSTKKSVQYDDVPEYKDRLNLSENYTLSISNARISDEKRFVCMLVTEDNVFEAPTIVKVFKQPSKPEIVSKALFLETEQLKKLGDCISEDSYPDGNITWYRNGKVLHPLEGAVVIIFKKEMDPVTQLYTMTSTLEYKTTKADIQMPFTCSVTYYGPSGQKTIHSEQAVFDIYYPTEQVTIQVLPPKNAIKEGDNITLKCLGNGNPPPEEFLFYLPGQPEGIRSSNTYTLTDVRRNATGDYKCSLIDKKSMIASTAITVHYLDLSLNPSGEVTRQIGDALPVSCTISASRNATVVWMKDNIRLRSSPSFSSLHYQDAGNYVCETALQEVEGLKKRESLTLIVEGKPQIKMTKKTDPSGLSKTIICHVEGFPKPAIQWTITGSGSVINQTEESPYINGRYYSKIIISPEENVTLTCTAENQLERTVNSLNVSANENREKVNDQAKLIVGIVVGLLLAALVAGVVYWLYMKKSKTASKHVNKDLGNMEENKKLEENNHKTEA

SEQ ID NO: 7 - DNA, 4816 bp, Homo sapiens
  source 1..4816: mol_type = genomic DNA
  CDS 521..4198
gttcccggatttttgtgggcgcctgccccgcccctcgtccccctgctgtgtccatatatcgaggcgatagggttaagggaaggcggacgcctgatgggttaatgagcaaactgaagtgttttccatgatcttttttgagtcgcaattgaagtaccacctcccgagggtgattgcttccccatgcggggtagaacctttgctgtcctgttcaccactctacctccagcacagaatttggcttatgcctactcaatgtgaagatgatgaggatgaaaacctttgtgatgatccacttccacttaatgaatggtggcaaagcaaagctatattcaagaccacatgcaaagctactccctgagcaaagagtcacagataaaacgggggcaccagtagaatggccaggacaaacgcagtgcagcacagagactcagaccctggcagccatgcctgcgcaggcagtgatgagagtgacatgtactgttgtggacatgcacaaaagtgagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagcta

SEQ ID NO: 8 - protein, 1225 aa, Homo sapiens
MKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 9 - DNA, 1321 bp, Homo sapiens
  source 1..1321: mol_type = genomic DNA
  CDS 185..871
cttttcccggcacatgcgcaccgcagcgggtcgcgcgccctaaggagtggcactttttaaaagtgcagccggagaccagcctacagccgcctgcatctgtatccagcgccaggtcccgccagtcccagctgcgcgcgccccccagtcccgcacccgttcggcccaggctaagttagccctcaccatgccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctagagtcagcttacatccctgagcaggaaagtttacccatgaagattggtgggattttttgtttgtttgttttgttttgtttgttgtttgttgtttgtttttttgccactaattttagtattcattctgcattgctagataaaagctgaagttactttatgtttgtcttttaatgcttcattcaatattgacatttgtagttgagcggggggtttggtttgctttggtttatattttttcagttgtttgtttttgcttgttatattaagcagaaatcctgcaatgaaaggtactatatttgctagactctagacaagatattgtacataaaagaatttttttgtctttaaatagatacaaatgtctatcaactttaatcaagttgtaacttatattgaagacaatttgatacataataaaaaattatgacaatgtcctggactggtaaaaaaa

SEQ ID NO: 10 - protein, 228 aa, Homo sapiens
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 11 - DNA, 4267 bp, Homo sapiens
  source 1..4267: mol_type = genomic DNA
  CDS 69..3218
attcgcctctgggaggtttaggaagcggctccgggtcggtggccccaggacagggaagagcgggcgctatggggagccggacgccagagtcccctctccacgccgtgcagctgcgctggggcccccggcgccgacccccgctgctgccgctgctgttgctgctgctgccgccgccacccagggtcgggggcttcaacttagacgcggaggccccagcagtactctcggggcccccgggctccttcttcggattctcagtggagttttaccggccgggaacagacggggtcagtgtgctggtgggagcacccaaggctaataccagccagccaggagtgctgcagggtggtgctgtctacctctgtccttggggtgccagccccacacagtgcacccccattgaatttgacagcaaaggctctcggctcctggagtcctcactgtccagctcagagggagaggagcctgtggagtacaagtccttgcagtggttcggggcaacagttcgagcccatggctcctccatcttggcatgcgctccactgtacagctggcgcacagagaaggagccactgagcgaccccgtgggcacctgctacctctccacagataacttcacccgaattctggagtatgcaccctgccgctcagatttcagctgggcagcaggacagggttactgccaaggaggcttcagtgccgagttcaccaagactggccgtgtggttttaggtggaccaggaagctatttctggcaaggccagatcctgtctgccactcaggagcagattgcagaatcttattaccccgagtacctgatcaacctggttcaggggcagctgcagactcgccaggccagttccatctatgatgacagctacctaggatactctgtggctgttggtgaattcagtggtgatgacacagaagactttgttgctggtgtgcccaaagggaacctcacttacggctatgtcaccatccttaatggctcagacattcgatccctctacaacttctcaggggaacagatggcctcctactttggctatgcagtggccgccacagacgtcaatggggacgggctggatgacttgctggtgggggcacccctgctcatggatcggacccctgacgggcggcctcaggaggtgggcagggtctacgtctacctgcagcacccagccggcatagagcccacgcccacccttaccctcactggccatgatgagtttggccgatttggcagctccttgacccccctgggggacctggaccaggatggctacaatgatgtggccatcggggctccctttggtggggagacccagcagggagtagtgtttgtatttcctgggggcccaggagggctgggctctaagccttcccaggttctgcagcccctgtgggcagccagccacaccccagacttctttggctctgcccttcgaggaggccgagacctggatggcaatggatatcctgatctgattgtggggtcctttggtgtggacaaggctgtggtatacaggggccgccccatcgtgtccgctagtgcctccctcaccatcttccccgccatgttcaacccagaggagcggagctgcagcttagaggggaaccctgtggcctgcatcaaccttagcttctgcctcaatgcttctggaaaacacgttgctgactccattggtttcacagtggaacttcagctggactggcagaagcagaagggaggggtacggcgggcactgttcctggcctccaggcaggcaaccctgacccagaccctgctcatccagaatggggctcgagaggattgcagagagatgaagatctacctcaggaacgagtcagaatttcgagacaaactctcgccgattcacatcgctctcaacttctccttggacccccaagccccagtggacagccacggcctcaggccagccctacattatcagagcaagagccggatagaggacaaggctcagatcttgctggactgtggagaagacaacatctgtgtgcctgacctgcagctggaagtgtttggggagcagaaccatgtgtacctgggtgacaagaatgccctgaacctcactttccatgcccagaatgtgggtgagggtggcgcctatgaggctgagcttcgggtcaccgcccctccagaggctgagtactcaggactcgtcagacacccagggaacttctccagcctgagctgtgactactttgccgtgaaccagagccgcctgctggtgtgtgacctgggcaaccccatgaaggcaggagccagtctgtggggtggccttcggtttacagtccctcatctccgggacactaagaaaaccatccagtttgacttccagatcctcagcaagaatctcaacaactcgcaaagcgacgtggtttcctttcggctctccgtggaggctcaggcccaggtcaccctgaacggtgtctccaagcctgaggcagtgctattcccagtaagcgactggcatccccgagaccagcctcagaaggaggaggacctgggacctgctgtccaccatgtctatgagctcatcaaccaaggccccagctccattagccagggtgtgctggaactcagctgtccccaggctctggaaggtcagcagctcctatatgtgaccagagttacgggactcaactgcaccaccaatcaccccattaacccaaagggcctggagttggatcccgagggttccctgcaccaccagcaaaaacgggaagctccaagccgcagctctgcttcctcgggacctcagatcctgaaatgcccggaggctgagtgtttcaggctgcgctgtgagctcgggcccctgcaccaacaagagagccaaagtctgcagttgcatttccgagtctgggccaagactttcttgcagcgggagcaccagccatttagcctgcagtgtgaggctgtgtacaaagccctgaagatgccctaccgaatcctgcctcggcagctgccccaaaaagagcgtcaggtggccacagctgtgcaatggaccaaggcagaaggcagctatggcgtcccactgtggatcatcatcctagccatcctgtttggcctcctgctcctaggtctactcatctacatcctctacaagcttggattcttcaaacgctccctcccatatggcaccgccatggaaaaagctcagctcaagcctccagccacctctgatgcctgagtcctcccaatttcagactcccattcctgaagaaccagtccccccaccctcattctactgaaaaggaggggtctgggtacttcttgaaggtgctgacggccagggagaagctcctctccccagcccagagacatacttgaagggccagagccaggggggtgaggagctggggatccctcccccccatgcactgtgaaggacccttgtttacacataccctcttcatggatgggggaactcagatccagggacagaggccccagcctccctgaagcctttgcattttggagagtttcctgaaacaacttggaaagataactaggaaatccattcacagttctttgggccagacatgccacaaggacttcctgtccagctccaacctgcaaagatctgtcctcagccttgccagagatccaaaagaagcccccagctaagaacctggaacttggggagttaagacctggcagctctggacagccccaccctggtgggccaacaaagaacactaactatgcatggtgccccaggaccagctcaggacagatgccacacaaggatagatgctggcccagggcccagagcccagctccaaggggaatcagaactcaaatggggccagatccagcctggggtctggagttgatctggaacccagactcagacattggcacctaatccaggcagatccaggactatatttgggcctgctccagacctgatcctggaggcccagttcaccctgatttaggagaagccaggaatttcccaggaccctgaaggggccatgatggcaacagatctggaacctcagcctggccagacacaggccctccctgttccccagagaaaggggagcccactgtcctgggcctgcagaatttgggttctgcctgccagctgcactgatgctgcccctcatctctctgcccaacccttccctcaccttggcaccagacacccaggacttatttaaactctgttgcaagtgcaataaatctgacccagtgcccccactgaccagaactagaaaaaaaaaaaaaaaaaaa

SEQ ID NO: 12 - protein, 1049 aa, Homo sapiens
MGSRTPESPLHAVQLRWGPRRRPPLLPLLLLLLPPPPRVGGFNLDAEAPAVLSGPPGSFFGFSVEFYRPGTDGVSVLVGAPKANTSQPGVLQGGAVYLCPWGASPTQCTPIEFDSKGSRLLESSLSSSEGEEPVEYKSLQWFGATVRAHGSSILACAPLYSWRTEKEPLSDPVGTCYLSTDNFTRILEYAPCRSDFSWAAGQGYCQGGFSAEFTKTGRVVLGGPGSYFWQGQILSATQEQIAESYYPEYLINLVQGQLQTRQASSIYDDSYLGYSVAVGEFSGDDTEDFVAGVPKGNLTYGYVTILNGSDIRSLYNFSGEQMASYFGYAVAATDVNGDGLDDLLVGAPLLMDRTPDGRPQEVGRVYVYLQHPAGIEPTPTLTLTGHDEFGRFGSSLTPLGDLDQDGYNDVAIGAPFGGETQQGVVFVFPGGPGGLGSKPSQVLQPLWAASHTPDFFGSALRGGRDLDGNGYPDLIVGSFGVDKAVVYRGRPIVSASASLTIFPAMFNPEERSCSLEGNPVACINLSFCLNASGKHVADSIGFTVELQLDWQKQKGGVRRALFLASRQATLTQTLLIQNGAREDCREMKIYLRNESEFRDKLSPIHIALNFSLDPQAPVDSHGLRPALHYQSKSRIEDKAQILLDCGEDNICVPDLQLEVFGEQNHVYLGDKNALNLTFHAQNVGEGGAYEAELRVTAPPEAEYSGLVRHPGNFSSLSCDYFAVNQSRLLVCDLGNPMKAGASLWGGLRFTVPHLRDTKKTIQFDFQILSKNLNNSQSDVVSFRLSVEAQAQVTLNGVSKPEAVLFPVSDWHPRDQPQKEEDLGPAVHHVYELINQGPSSISQGVLELSCPQALEGQQLLYVTRVTGLNCTTNHPINPKGLELDPEGSLHHQQKREAPSRSSASSGPQILKCPEAECFRLRCELGPLHQQESQSLQLHFRVWAKTFLQREHQPFSLQCEAVYKALKMPYRILPRQLPQKERQVATAVQWTKAEGSYGVPLWIIILAILFGLLLLGLLIYILYKLGFFKRSLPYGTAMEKAQLKPPATSDA

SEQ ID NO: 13 - DNA, 5748 bp, Homo sapiens
  source 1..5748: mol_type = genomic DNA
  CDS 435..2663
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacagaaaaaaaaaaaaaaaaa

SEQ ID NO: 14 - protein, 742 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLNGEASKSQEMVHLVNKESSETPDQFMTADETRNLQNVDMKIGV

SEQ ID NO: 15 - DNA, 3936 bp, Homo sapiens
  source 1..3936: mol_type = genomic DNA
  CDS 557..2131
actcctcctctctgcccctcagctcgctcatctttcttcccgccccctctcttttccttctttggttctttgaagtgatgagctagcgcaaccacaaaccatacattccttttgtagaaaaacccgtgcctcgaatgaggcgagactcagagaggacccaggcgcggggcggacccctccaattccttcctcgcgcccccgaaagagcggcgcaccagcagccgaactgccggcgcccaggctccctggtccggccgggatgcggccggtacccgctccccgccgggaacaacctctccactcttcctgcagggagctggtgccagccgacagccgcgccagggccgctccgggtaccagggtcggatcgggtgacgtcgcgaacttgcgcctggccgccaagccggcctccaggctgaagaaggacccgccccggccttgacccgggccccgcccctccagccggggcaccgagccccggccctagctgctcgcccctactcgccggcactcgcccggctcgcccgctttcgcacccagttcacgcgccacagctatgtgtccccgagccgcgcgggcgcccgcgacgctactcctcgccctgggcgcggtgctgtggcctgcggctggcgcctgggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggcactgggaaatcatgaatttgataatggtgtggaaggactgatcgagccactcctcaaagaggccaaatttccaattctgagtgcaaacattaaagcaaaggggccactagcatctcaaatatcaggactttatttgccatataaagttcttcctgttggtgatgaagttgtgggaatcgttggatacacttccaaagaaaccccttttctctcaaatccagggacaaatttagtgtttgaagatgaaatcactgcattacaacctgaagtagataagttaaaaactctaaatgtgaacaaaattattgcactgggacattcgggttttgaaatggataaactcatcgctcagaaagtgaggggtgtggacgtcgtggtgggaggacactccaacacatttctttacacaggcaatccaccttccaaagaggtgcctgctgggaagtacccattcatagtcacttctgatgatgggcggaaggttcctgtagtccaggcctatgcttttggcaaatacctaggctatctgaagatcgagtttgatgaaagaggaaacgtcatctcttcccatggaaatcccattcttctaaacagcagcattcctgaagatccaagcataaaagcagacattaacaaatggaggataaaattggataattattctacccaggaattagggaaaacaattgtctatctggatggctcctctcaatcatgccgctttagagaatgcaacatgggcaacctgatttgtgatgcaatgattaacaacaacctgagacacacggatgaaatgttctggaaccacgtatccatgtgcattttaaatggaggtggtatccggtcgcccattgatgaacgcaacaatggaatccatgtggtgtatgatctttcccgaaaacctggagacagagtagtcaaattagatgttctttgcaccaagtgtcgagtgcccagttatgaccctctcaaaatggacgaggtatataaggtgatcctcccaaacttcctggccaatggtggagatgggttccagatgataaaagatgaattattaagacatgactctggtgaccaagatatcaacgtggtttctacatatatctccaaaatgaaagtaatttatccagcagttgaaggtcggatcaagttttccacaggaagtcactgccatggaagcttttctttaatatttctttcactttgggcagtgatctttgttttataccaatagccaaaaattctccttgcctttaatgtgtgaaactgcattttttcaagtgagattcaaatctgccttttaggacctggctttgtgacagcaaaaaccatctttacaggctcctagaagctgaaggttagagcattataaaatgaagagacagacatgattactcagggtcagcaacctagtgagttagaaaaaaaattaacatagggccctataaggagaaagccaactatgttaagtttacgtgtccaaattttaatgaaattttactaacaattttaaaccatatttttcttcttcatatccatttctaatccatcaaacagcttatgtttacataaaattttatcattcacaaggaagttttaagcacactgtctcatttgatatccacaacttatttttggtaggaaagagagatgtttttcccacctgtcagatgaaaaaactgaagctcaaaaagggttgacttgaccatacagctaatgctgacagatccaagacctagacctaggtcttttgaactcaagtccagcattctcaactatatcaagttactgttcagaatacttaatatctcctctcttcataattatcaatagccccaagctcatggatgacaaatctctgctttatttcttgtctctattttttcactttatagctcctgttataatagcaagtttaatggtataaacacaggataccatcctctcttgcaacacccatgtgcctttgatgagtcaggtagcaagctgtagtagataatgagaaaggccagaggctgcaaaagacagtcaaaggacacgagagaaaggaaggggaagaacaggactccaggactgttttatattatagaaaagcaagagctaaagagcatttacacatgttaaacagatacttgttaagcatagtgcctgacacacggcattagctgttattttatgagattccatcagctctgcctctgtcctctttcttctaacatgaaggtatcatgagaagagaaccttctaacataagctgtaattctaaacctgcacttgtccctctccagcaagaggctagcactgaattcattctactcatactacacacccagttatggaatgtccagagttctcgaagaaaataaatgactttaggaagaggtatacattttttaagtcgctctgcctccaaatctgaacagtcactgtaaatcattcttaagcccagatatgagaacttctgctggaaagtgggaccctctgagtgggtggtcagaaaatacccatgctgatgaaatgacctatgcccaaagaacaaatacttaacgtgggagtggaaccacatgagcctgctcagctctgcataagtaattcaagaaatgggaggcttcaccttaaaaacagtgtgcaaatggcagctagaggttttgataggaagtatgtttgtttcttagtgtttacaaatattaagtactcttgatacaaaatatacttttaaacttcataacctttttataaaagttgttgcagcaaaataatagcctcggttctatgcatatatggattagctataaaaaatgtcaataagattgtacaaggaaaattagagaaagtcacatttagggtttattttttacacttggccagtaaaatagggtaaatcctattagaattttttaaagaactttttttaagtttcctaaatctgtgtgtgtattgtgaagtggtataagaaatgactttgaaccactttgcaattgtagattcccaacaataaaattgaagataagctctttggtcaaaaaaaaaaaaaaaaaa

SEQ ID NO: 16 - protein, 524 aa, Homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ

SEQ ID NO: 17 - DNA, 23 bp, synthetic construct
  misc_feature 1..23: note = CD166-F primer
  source 1..23: mol_type = other DNA
ccccagaggaatttttgttttac

SEQ ID NO: 18 - DNA, 23 bp, synthetic construct
  misc_feature 1..23: note = CD166-R primer
  source 1..23: mol_type = other DNA
agcctgatgttatctttcatcca

SEQ ID NO: 19 - DNA, 22 bp, synthetic construct
  misc_feature 1..22: note = CD73-F primer
  source 1..22: mol_type = other DNA
gttcctgtagtccaggcctatg

SEQ ID NO: 20 - DNA, 22 bp, synthetic construct
  misc_feature 1..22: note = CD73-R primer
  source 1..22: mol_type = other DNA
acatttcatccgtgtgtctcag

SEQ ID NO: 21 - DNA, 20 bp, synthetic construct
  misc_feature 1..20: note = GAPDH-F primer
  source 1..20: mol_type = other DNA
gagtcaacggatttggtcgt

SEQ ID NO: 22 - DNA, 20 bp, synthetic construct
  misc_feature 1..20: note = GAPDH-R primer
  source 1..20: mol_type = other DNA
ttgattttggagggatctcg

(19) United States
(12) Patent Application Publication
(10) Pub. No.: US 2024/0402171 A1
(43) Pub. Date: Dec. 5, 2024

(54) CORNEAL ENDOTHELIAL CELL MARKER

(71) Applicants: KYOTO PREFECTURAL PUBLIC UNIVERSITY CORPORATION, Kyoto-Shi (JP); ACTUALEYES INC., Kyotanabe-City (JP); AURION BIOTECH, INC., Seattle, WA (US)

(72) Inventors: Noriko Koizumi, Kyotanabe (JP); Naoki Okumura, Kyotanabe (JP); Hiroatsu Hirano, Kyotanabe (JP); Shigeru Kinoshita, Kyoto (JP); Morio Ueno, Kyoto (JP)

(73) Assignees: KYOTO PREFECTURAL PUBLIC UNIVERSITY CORPORATION, Kyoto-Shi (JP); ACTUALEYES INC., Kyotanabe-City (JP); AURION BIOTECH, INC., Seattle, WA (US)

(21) Appl. No.: 18/815,522

(22) Filed: Aug. 26, 2024

Related U.S. Application Data
(63) Continuation of application No. 17/133,326, filed on Dec. 23, 2020, now Pat. No. 12,072,334.
Application No. 17/133,326 is a continuation of application No. 14/908,036, filed on Jan. 27, 2016, now Pat. No. 10,908,161.

(30) Foreign Application Priority Data
Jul. 30, 2013 (JP) 2013-157597

(51) Int. Cl. G01N 33/569; A61K 35/30; C12N 5/079

(52) U.S. Cl. CPC G01N 33/56966; A61K 35/30; C12N 5/0621; G01N 2333/705; G01N 2333/70596

(57) ABSTRACT

The purpose of the present invention is to provide a method of purification and preparation of cultured corneal endothelial cells, and in particular, to provide cell surface markers for use in corneal endothelial cells not including transformed cells. Provided are cell markers for distinguishing normal cells and transformed cells, in particular normal and transformed corneal endothelium cells. These cell markers relate to specific cell surface markers, for example, to a normal corneal endothelial surface marker such as CD166, and a transformed cell surface marker such as CD73. By using the transformed cell surface marker such as CD73 to remove transformed cells by sorting, it becomes possible to improve purity of a normal cultured corneal endothelium. By using normal corneal endothelial surface marker such as CD166, or by combined use with the transformed cell surface marker, it becomes possible to provide a means for verifying the purity of a prepared corneal endothelium.

### CROSS-REFERENCE TO RELATED APPLICATIONS

The present application is a continuation of U.S. application Ser. No. 17/133,326, filed Dec. 23, 2020, now U.S. Pat. No. 12,072,334, which is a continuation of U.S. application Ser. No. 14/908,036, filed Jan. 27, 2016, now U.S. Pat. No. 10,908,161, which is the U.S. National Stage of International Application No. PCT/JP2014/070412, filed Jul. 28, 2014, and claims priority to Japanese Patent Application No. 2013-157597, filed Jul. 30, 2013.

### SEQUENCE LISTING

The present application contains a sequence listing which has been submitted electronically in XML format and is hereby incorporated by reference in its entirely. Said XML copy, created on Jul. 22, 2024, is named “DS006-2US_Sequence Listing.xml” and is 58.8 KB in size.

### TECHNICAL FIELD

The present invention relates to a cell, particularly a differentiated marker in the ophthalmological region, and particularly a corneal endothelial cell marker.

### BACKGROUND ART

Visual information is recognized when light transmitted into the cornea, which is a transparent tissue at the front-most part of an eye ball, reaches the retina and excites nerve cells of the retina and a generated electric signal is transmitted through the optic nerve to the visual cortex of the cerebrum. To attain good vision, it is necessary that the cornea is transparent. The transparency of the cornea is maintained by maintaining constant water content with pumping and barrier functions of corneal endothelial cells.

The cornea is a transparent tissue positioned in the front side of an eyeball and having a three layer structure of mainly a corneal epithelial cell layer, a corneal stroma layer, and a corneal endothelial layer. The corneal endothelial layer is a single-layer cell layer present in the deeper part of the cornea. The corneal endothelial cell layer has barrier and pumping functions and plays a role in maintaining transparency of the cornea by maintaining a constant amount of moisture in the cornea. Further, it is known that corneal endothelial cells do not grow in a living body even if they are damaged. In addition, it is known that a decrease in the number of corneal endothelial cells from damage due to trauma, disease or the like to the cells leads to severe visual impairment.

Human corneal endothelial cells are present at a density of about 3000 per 1 mm² at birth. However, once damaged, corneal endothelial cells do not have the ability to regenerate. Corneal endothelial dystrophy or bullous keratopathy induced by dysfunction of the corneal endothelium due to various causes results in edema or turbidity of the cornea, leading to significant deterioration in vision. Currently, penetrating keratoplasty is performed to transplant the entire three layer structure consisting of epithelium, stroma, and endothelium of the cornea for bullous keratopathy. However, cornea donation is insufficient in Japan. While there are about 2600 patients waiting for cornea transplantation, the number of corneal transplantations cases performed domestically with a donor cornea is about 1700 annually.

In recent years, the concept of “part transplantation” for transplanting only damaged tissue has drawn attention in order to alleviate the risk of post-operative complication or rejection and to attain better visual function. Among corneal transplantations, deep lamellar keratoplasty, which is a transplantation of stromal tissue, Descemet's stripping automated endothelial keratoplasty, which is a transplantation of corneal endothelial tissue, and the like have been performed. Further, cultured mucosal epithelium transplantation for transplanting corneal epithelium or oral mucosa cultured in vitro to replace the corneal epithelium is already in clinical application. Similarly, a method of transplanting corneal endothelium cultured in vitro also has been investigated.

A method of regenerating and utilizing corneal endothelial cells is being investigated, but there is hardly any marker for identifying regenerated corneal endothelial cells.

Although Patent Literature 1 has descriptions on corneal endothelial cell markers, they are different from the markers of the present invention. Although Patent Literature 2 discloses corneal endothelial cell markers, none are surface antigens. Although Patent Literature 3 discloses the concept of identifying corneal endothelial cells, the marker used is a neural crest marker.

Although Non Patent Literature 1 has descriptions on identification of corneal endothelial cell-specific cell surface markers, there is no specific disclosure. Although Non Patent Literature 2 discloses a corneal endothelial cell marker, the marker is not a surface antigen. Although Non Patent Literature 3 has descriptions on epithelial cell or endothelial cell markers, there is no specific description related to corneal endothelial cells. Non Patent Literature 4 is a document describing a marker for corneal stroma cells, which does not have a description related to corneal endothelial cells. Although Non Patent Literature 5 discloses corneal endothelial cell markers, none are surface antigens. Although Non Patent Literature 6 discloses corneal endothelial cell markers, none are surface antigens. Although Non Patent Literature 7 discloses corneal endothelial cell markers, none are surface antigens. Although Non Patent Literature 8 discloses corneal endothelial cell markers, there is no specific disclosure. Although Non Patent Literature 9 discloses corneal endothelial cell markers, none are surface antigens.

In this manner, the state of surface molecules for determining whether transformation of a corneal endothelium is normal has not been elucidated.

### CITATION LIST

### Patent Literature

[PTL 1] International Publication No. WO 2013/086236
[PTL 2] International Publication No. WO 2013/051722
[PTL 3] International Publication No. WO 2013/012087

### Non Patent Literature

[NPL 1] Saisei Iryo [Regenerative Medicine]: Vol. 9, Extra issue, Page. 283 (2010.02.05)
[NPL 2] The Proceedings of the General Meeting of the Japan Cornea Society/Keratoplasty Society of Japan Program: Vol. 35th-27th, Page. 75 (2011)
[NPL 3] Am J Pathol. 2011 February; 178 (2) 572-9.
[NPL 4] Cytotherapy. 2007; 9 (3): 252-8
[NPL 5] Japanese Journal of Ophthalmology, Vol. 117, Extra edition, Page. 378 (2013.03.04)
[NPL 6] Kunzevitzky, N J, et al. ARVO E-Abstract1698, Poster Board Number: D0333, 2013
[NPL 7] Mehta J S, et al. ARVO E-Abstract 2198, 2013
[NPL 8] Saisei Iryo [Regenerative Medicine] Vol. 10, Extra edition, Page. 201 (2011.02.01)
[NPL 9] Japanese Journal of Ophthalmology, Vol. 105, Extra edition, Page. 195 (2001.03.15)

### SUMMARY OF INVENTION

### Solution to Problem

The present invention provides a marker for identifying corneal cells, especially normal and transformed corneal endothelial cells. Thus, the present invention also provides the following.

(1) A marker for identifying a normal cell and a transformed cell in corneal endothelial cells, comprising at least one marker selected from
a normal corneal endothelial cell marker comprising a nucleic acid of a gene selected from the group consisting of CD166, HLA-A2, CD66a, CD66c, CD66d, CD66e, CD98, CD59, CD54, CD340, CD47, EGF-R, CD29, CD74, CD165, CD221, CD49a, SSEA-4, CD130, and CD49f and/or a protein encoded by the nucleic acid, and
a transformed corneal endothelial cell marker comprising a nucleic acid of a gene selected from the group consisting of CD26, CD9, CD49b, CD49e, CD13, CD99, CD105, CD63, CD58, CD201, CD56, CD44, CD55, CD71, CD73, CD91, HLA-DQ, CD164, CD49d, CD49c, CD90, MICA/B, CD46, CD140b, CD146, CD147, CD81, CD151, CD200, and CD10 and/or a protein encoded by the nucleic acid.

(2) The marker of item 1, wherein the marker is a cell surface marker.

(3) The marker of item 1 or 2, wherein the marker comprises at least one transformed corneal endothelial cell marker and at least one normal corneal endothelial cell marker.

(4) The marker according to any one of items 1-3, wherein the marker comprises at least one molecule selected from the group consisting of CD98, CD166, and CD340 as the normal corneal endothelial cell marker.

(5) The marker according to any one of items 1-4, wherein the marker comprises at least one molecule selected from the group consisting of CD9, CD49e, CD44, and CD73 as the transformed corneal endothelial cell marker.

(6) The marker according to any one of items 1-5, wherein the marker comprises at least one molecule selected from the group consisting of CD98, CD166, and CD340 as the normal corneal endothelial cell marker and at least one molecule selected from the group consisting of CD9, CD49e, CD44, and CD73 as the transformed corneal endothelial cell marker.

(7) The marker according to any one of items 1-6, wherein the marker comprises at least one marker selected from the group consisting of CD166 and CD73.

(8) A detection agent for identifying a normal corneal endothelial cell and a transformed corneal endothelial cell, comprising a substance that binds to the marker according to any one of items 1-7.

(9) A method of using the marker according to any one of items 1-7 as an indicator for identifying a normal corneal endothelial cell and a transformed corneal endothelial cell.

(10) A method of preparing a corneal endothelial cell sample with an elevated ratio of normal corneal endothelial cells, comprising,
a) providing a sample comprising corneal endothelial cells, and
b) elevating a ratio of normal corneal endothelial cells while using the marker according to any one of items 1-7 as an indicator in the sample.

(11) An agent for determining purity when making a sample comprising corneal endothelial cells, comprising the marker according to any one of items 1-7 or a substance that binds to the marker.

(12) A method of using the marker according to any one of items 1-7 as an indicator for determining purity when making a sample comprising corneal endothelial cells.

(13) A method of preparing purified corneal endothelial cells, wherein the method comprises:
inducing corneal endothelial cells from undifferentiated cells; and
elevating a ratio of the corneal endothelial cells while using the marker according to any one of items 1-7 as an indicator.

(14) The method of item 13, wherein the undifferentiated cells are induced pluripotent stem (iPS) cells or embryonic stem (ES) cells.

(15) A composition comprising a cell prepared by the method of item 10 or 13.

(16) The composition of item 15 for corneal therapy.

(17) The composition of item 16, wherein a subject of the corneal therapy includes corneal endothelial diseases.

Alternatively, the present invention may provide the following.

(A1) A marker for identifying a normal cell and a transformed cell in corneal endothelial cells, comprising at least one marker selected from
a normal corneal endothelial cell marker comprising a nucleic acid of a gene selected from the group consisting of CD166, HLA-A2, CD66a, CD66c, CD66d, CD66e, CD98, CD59, CD54, CD340, CD47, EGF-R, CD29, CD74, CD165, CD221, CD49a, SSEA-4, CD130, and CD49f and/or a protein encoded by the nucleic acid, and
a transformed corneal endothelial cell marker comprising a nucleic acid of a gene selected from the group consisting of CD26, CD9, CD49b, CD49e, CD13, CD99, CD105, CD63, CD58, CD201, CD56, CD44, CD55, CD71, CD73, CD91, HLA-DQ, CD164, CD49d, CD49c, CD90, MICA/B, CD46, CD140b, CD146, CD147, CD81, CD151, CD200, and CD10 and/or a protein encoded by the nucleic acid.

(A2) The marker of item A1, wherein the marker is a cell surface marker.

(A3) The marker of item A1 or A2, wherein the marker comprises at least one transformed corneal endothelial cell marker and at least one normal corneal endothelial cell marker.

(A4) The marker according to any one of items A1-A3, wherein the marker comprises at least one molecule selected from the group consisting of CD98, CD166, and CD340 as the normal corneal endothelial cell marker.

(A5) The marker according to any one of items A1-A4, wherein the marker comprises at least one molecule selected from the group consisting of CD9, CD49e, CD44, and CD73 as the transformed corneal endothelial cell marker.

(A6) The marker according to any one of items A1-A5, wherein the marker comprises at least one molecule selected from the group consisting of CD98, CD166, and CD340 as the normal corneal endothelial cell marker and at least one molecule selected from the group consisting of CD9, CD49e, CD44, and CD73 as the transformed corneal endothelial cell marker.

(A7) The marker according to any one of items A1-A6, wherein the marker comprises at least one marker selected from the group consisting of CD166 and CD73.

(A8) A detection agent for identifying a normal corneal endothelial cell and a transformed corneal endothelial cell, comprising a substance that binds to the marker according to any one of items A1-A7.

(A9) An agent for diagnosis for identifying a normal corneal endothelium cell and a transformed corneal endothelium cell, comprising a substance that binds to the marker according to any one of items A1-A7.

(A10) The agent for diagnosis of item A9, wherein the corneal endothelial cells are cells present in a living body.

(A11) The agent for diagnosis of item A9 or A10, wherein the diagnosis comprises diagnosis of a corneal endothelial disease.

(A12) The agent for diagnosis of item A11, wherein the corneal endothelial disease is a disease associated with a fibroblast.

(A13) The agent for diagnosis of item A11 or A12, wherein the corneal endothelial disease is selected from the group consisting of bullous keratopathy, corneal endothelial disorders due to trauma or intraocular surgery, and corneal dystrophy including Fuchs corneal endothelial dystrophy and posterior polymorphous corneal endothelial dystrophy.

(A14) The agent for diagnosis according to any one of items A9-A13, wherein a transformed corneal endothelial cell is determined to be comprised when at least one of the transformed corneal endothelial cell marker is expressed.

(A15) An agent for diagnosis of a corneal endothelial cell, comprising a substance that binds to the marker according to any one of items A1-A7, wherein the corneal endothelial cell is diagnosed to be suffering from irreversible dystrophy when at least one of the transformed corneal endothelial cell marker is expressed.

(A16) The agent for diagnosis according to any one of items A9-A15, further comprising an agent for staining the substance that binds to the marker.

(A17) A method of using the marker according to any one of items A1-A7 as an indicator for identifying a normal corneal endothelial cell and a transformed corneal endothelial cell.

(A18) A method of preparing a corneal endothelial cell sample with an elevated ratio of normal corneal endothelial cells, comprising,
a) providing a sample comprising corneal endothelial cells, and
b) elevating a ratio of normal corneal endothelial cells while using the marker according to any one of items A1-A7 as an indicator in the sample.

(A19) An agent for determining purity when making a sample comprising corneal endothelial cells, comprising the marker according to any one of items A1-A7 or a substance that binds to the marker.

(A20) A method of using the marker according to any one of items A1-A7 as an indicator for determining purity when making a sample comprising corneal endothelial cells.

(A21) A method of preparing purified corneal endothelial cells, wherein the method comprises:
inducing corneal endothelial cells from undifferentiated cells; and
elevating a ratio of the corneal endothelial cells while using the marker according to any one of items A1-A7 as an indicator.

(A22) The method of item A21, wherein the undifferentiated cells are induced pluripotent stem (iPS) cells or embryonic stem (ES) cells.

(A23) A composition comprising a cell prepared by the method according to any one of items A18-A22.

(A24) The composition of item A23 for corneal therapy.

(A25) The composition of item 24A, wherein a subject of the corneal therapy includes corneal endothelial diseases.

(B8) A method of detection for identifying a normal corneal endothelial cell and a transformed corneal endothelial cell, comprising: applying a substance that binds to the marker according to any one of items A1-A7 to a sample comprising a corneal endothelial cell; and detecting a binding between the substance and the corneal endothelial cell.

(B9) A method of diagnosis of a corneal endothelial cell, comprising: applying a substance that binds to the marker according to any one of items A1-A7 to a sample comprising the corneal endothelial cell; and detecting a binding between the substance to the cell, wherein a normal corneal endothelial cell and a transformed corneal endothelial cell are identified by determining the presence of the binding.

(B10) The method of item B9, wherein the corneal endothelial cell is a cell present in an organism.

(B11) The method of item B9 or B10, wherein the diagnosis comprises diagnosis of a corneal endothelial disease.

(B12) The method of item B11, wherein the corneal endothelial disease is a disease associated with a fibroblast.

(B13) The method of item B11 or B12, wherein the corneal endothelial disease is selected from the group consisting of bullous keratopathy, corneal endothelial disorder due to trauma or intraocular surgery, and corneal dystrophy including Fuchs corneal endothelial dystrophy and posterior polymorphous corneal endothelial dystrophy.

(B14) The agent for diagnosis according to any one of items B9-B13, wherein the transformed corneal endothelial cell is determined to be comprised when at least one of the transformed corneal endothelial cell marker is expressed.

(B15) A method of diagnosis of a corneal endothelial cell, comprising: applying a substance that binds to the marker according to any one of items A1-A7 to a sample comprising a corneal endothelial cell; and detecting a binding between the substance and the corneal endothelial cell, wherein the corneal endothelial cell is diagnosed to be suffering from irreversible dystrophy when at least one of the transformed corneal endothelial cell marker is expressed.

(B16) The method according to any one of items B9-B15, wherein the substance that binds to the marker further comprises an agent for staining the substance that binds to the marker.

(B19) A method of determining purity when making a sample comprising a corneal endothelial cell, comprising: applying a substance that binds to the marker according to any one of items A1-A7 to a sample comprising the corneal endothelial cell; and detecting a binding between the substance and the cell, wherein a normal corneal endothelial cell and a transformed corneal endothelial cell are identified by determining the presence of the binding.

(B23) A method of therapy or prevention using a cell prepared by the method according to any one of items A18-A22.

(B24) The method of item B23, wherein the therapy or prevention is for a cornea.

(B25) The method of item B24, wherein a subject of the therapy and prevention includes corneal endothelial diseases.

In still another aspect, the present invention provides an agent for diagnosis, a detection kit, a diagnostic kit, a detection system, a diagnostic system or the like using the detection agent, marker or the like of the present invention.

In still another aspect, the present invention provides a therapeutic method, a prevention method, use or the like using the pharmaceutical composition, therapeutic agent or progression preventive agent of the present invention.

It is understood that the present invention can further combine and use one or more of the aforementioned features.

Further embodiments and advantages of the present invention are recognized by those skilled in the art by reading the understanding the following Detailed Description as needed.

### Advantageous Effects of Invention

The present invention can identify the cell differentiation capability of corneal endothelial cells to identify cells that are highly functional. As a result, it is possible to treat or prevent corneal endothelial disease or disorder that was previously impossible or difficult. Further, a method of determining purity when making a sample comprising a cell, a method of purifying a corneal endothelial cell, and a method of preparing a purified corneal endothelial cell are also provided.

### BRIEF DESCRIPTION OF DRAWINGS

FIG. 1 is a diagram related to solving problems in the production of cultured corneal endothelial cells. A novel surgical procedure for the transplantation of cultured corneal endothelial cells was established in monkey and rabbit models in order to establish novel therapy for corneal endothelial dysfunction. Conventional techniques culture monkey endothelial cells on a type I collagen sheet and transplant a cultured corneal endothelial sheet thereof in a corneal endothelial dysfunction monkey model or inject the cells as a cell suspension into the anterior chamber. The top row in the Figure describes a schematic flow chart for, from the left, separating human corneal endothelial cell from a donor cornea, culturing the cells, and transplanting the cell. The picture on the bottom left shows a corneal endothelium cultured into a normal form, and the bottom right shows a cell transformed by culturing.

FIG. 2 shows images from a phase difference microscope in cultured monkey corneal endothelial cells. The normal corneal endothelial cells shown on the left side show how the cells consist of polygonal, mainly hexagonal, cells. In contrast, the cells transformed into a fibroblast-like shape shown on the right side are not polygonal cells, but have an elongated fibroblast-like shape.

FIG. 3 shows results of flow cytometry analysis on candidate markers in cultured monkey corneal endothelial cells. The left side shows normal cells and the right side shows transformed cells. Further, the top row shows results for CD166 and the bottom row shows results for CD73. It is clearly confirmed from the results that CD166 is highly expressed in normal cells and CD73 is highly expressed in transformed cells.

FIG. 4 shows results of PCR analysis on candidate markers in cultured monkey corneal endothelial cells. The results are for, from the top, CD166, CD73, and GAPDH as a control. The left column is for normal cells and the right column is for transformed cells. It can be clearly confirmed from the results that especially CD166 is highly expressed in normal cells and CD73 is highly expressed in transformed cells. This is consistent with the results of flow cytometry analysis.

FIG. 5 shows images of immunostaining of candidate markers in cultured monkey corneal endothelial cells. The staining is performed using, from the left, CD166 and CD73. The top row shows normal corneal endothelial cells and the bottom row shows transformed cells. It can be seen from the results that CD166 is highly expressed in normal corneal endothelial cells and CD73 is highly expressed in transformed cells. The results are consistent with the results of flow cytometry analysis. The bar indicates 100 μm.

FIG. 6 shows results of images from a phase difference microscope in cultured monkey corneal endothelial cells. The top left shows normal corneal endothelial cells and the top right shows transformed cells. The bottom row shows a mixture of the normal cells and the transformed cells, which was then sorted and cultured depending on the level of CD73 expression. Cells with low level of CD73 expression exhibit a normal polygonal cell form. Meanwhile, cells with high level of CD73 expression exhibit a transformed fibroblast-like form.

FIG. 7 shows results of images of immortalized human corneal endothelial cells from a phase difference microscope. The left shows normal corneal endothelial cells and the middle shows transformed cells. The left shows a cultured mixture of the normal cells and transformed cells. The bar indicates 100 μm.

FIG. 8 examines the CD73 expression of a mixture of normal cells and transformed cells in the immortalized human corneal endothelial cells shown in FIG. 7 by flow cytometry. FIG. 8 shows that the results separate into a high expression peak and a low expression peak for CD73.

FIG. 9 shows a mixture of normal cells and transformed cells in the immortalized human corneal endothelial cells shown in FIG. 7 which is sorted and cultured depending on the CD73 expression by flow cytometry. Cells with low level of CD73 expression exhibit a normal polygonal cell form. In contrast, cells with high level of CD73 expression exhibit a transformed fibroblast-like form. Cells with high and low levels of CD73 expression cultured without sorting are partially comprised of normal cells and partially comprised of transformed cells.

FIG. 10 shows results of analyzing expression of candidate markers after sorting by CD73. The top row shows results after sorting cells with low level of CD73 expression and the bottom row shows results after sorting cells with high level of CD73 expression. The left shows staining by CD73 and the right shows staining by CD166. While cells with low level of CD73 expression had a high level of CD166 expression in immunostaining, cells with high level of CD73 expression had low level of CD166 expression in immunostaining.

FIG. 11 shows results of analyzing expression of function associated markers of corneal endothelial cells after sorting by CD73. The top row shows results after sorting cells with low level of CD73 expression and the bottom row shows results after sorting cells with high level of CD73 expression. The left side shows expression of ZO-1 and the right side shows expression of Na⁺/K⁺-ATPase. Expression of ZO-1 and Na⁺/K⁺-ATPase was observed in cells with low level of CD73 expression in immunostaining.

### DESCRIPTION OF EMBODIMENTS

The present invention is described hereinafter. Throughout the entire specification, a singular expression should be understood as encompassing the concept thereof in the plural form unless specifically noted otherwise. Thus, singular articles (e.g., “a”, “an”, “the” and the like in case of English) should also be understood as encompassing the concept thereof in the plural form unless specifically noted otherwise. Further, the terms used herein should be understood as being used in the meaning that is commonly used in the art, unless specifically noted otherwise. Thus, unless defined otherwise, all terminologies and scientific technical terms that are used herein have the same meaning as the terms commonly understood by those skilled in the art to which the present invention pertains. In case of a contradiction, the present specification (including the definitions) takes precedence.

As used herein, “CD98” is one type of CD protein and a glycoprotein, which is a heterodimer comprised of SLC7A5 (light subunit protein of CD98; also called LAT1, E16, D16S469E, MPE16 or the like. Gene ID (Entrez): 8140 NM 003486; NP 003477) and SLC3A2 (solute carrier family 3 (dibasic and neutral amino acid transport activator) member 2, 4F2hc/CD98; heavy subunit protein of CD98; also called MDU1, 4T2HC, 4F2, or NACAE. The transporter serves a role in intracellular calcium level regulation and transportation of L-amino acid. Gene ID (Entrez): 6520; NM 001013251 (NM 001012661) and NP 001012680) forming a large neutral amino acid transporter 1; LAT1). SEQ ID NOS: 1-4 are representative sequences thereof (SLC3A2=SEQ ID NOS: 1-2; SLC7A5=SEQ ID NOS: 3-4; nucleic acid sequence and amino acid sequence, respectively).

As used herein, “CD166” is a type I transmembrane protein known as activated leukocyte cell adhesion molecule (ALCAM). CD166 is an immunoglobulin superfamily molecule comprising 10 N-linked glycosylation sites. CD166 is comprised of 5 extracellular Ig-like domains, 32 amino acid intracellular terminals and a short transmembrane region with a molecular weight of 100-105 kDa. CD166 is an adhesion molecule forming a binding with a CD6 molecule or a homophilic binding of ALCAM-ALCAM. CD166 is also called ALCAM; CD166; or MEMD. Please also refer to documents PMID: 23056608 and PMID: 18334960. Various accession numbers are Gene ID (Entrez): 214 (human), NM_001243280 (mRNA), and NP_001230209 (protein). SEQ ID NOs: 5-6 are representative sequences thereof (nucleic acid sequence and amino acid sequence, respectively).

As used herein, “CD340” is one type of CD known as HER2 or neu oncogene. CD340 is a receptor tyrosine kinase, which is a glycoprotein of about 185 kDa present on the cell surface. CD340 is also called NEU; NGL; HER2; TKR1; CD340; HER-2; MLN 19; or HER-2/neu. CD340 is an oncogene homolog (avian) derived from an erythroblast leukemia ERBB2: virus v-erb-b2. This gene encodes a member of an epithelial growth factor (EGF) receptor family of receptor tyrosine kinases. Since this protein does not have its own ligand binding domain, it is understood that the protein cannot bind to a growth factor. However, it is understood that the protein binds to a member of another ligand binding EGF receptor family member to form a heterodimer to stabilize the ligand binding and enhances kinase mediated activation of a downstream signaling pathway (e.g., mitosis activated protein kinase, phosphatidylinositol-3 kinase and the like). Please refer to the document PMID: 15557433. Entrez Gene ID: (Hu) 2064 and NM_0010005862.1. SEQ ID NOs: 7-8 are representative sequences thereof (nucleic acid sequence and amino acid sequence, respectively).

As used herein, “CD9” is a member of the transmembrane 4 superfamily and is also called tetraspanin. CD9 is also called CD9; BTCC-1; DRAP-27; MIC3; MRP-1; TSPAN-29; or TSPAN29. Tetraspanin is a cell surface glycoprotein with a transmembrane 4 domain and forms a multimer complex with another cell surface protein. A human amniotic epithelial cell (hHEAC) expresses CD9, CD44, CD73, and CD90, as well as negligible levels of CD31, CD34, CD45, and CD117. Please refer to PMID: 21166885. A CD9 gene encodes 1.4 kb of mRNA and is detectably and dominantly expressed in a cornea and at a low level in the ciliary epithelium, retina, iris, and lens. Please refer to the document PMID: 1339429. CD9 is Entrez Gene ID: 928, RefSeq (mRNA): NM_001769 (Hu), RefSeq (protein): NP_001760 (Hu). SEQ ID NOs: 9-10 are representative sequences thereof (nucleic acid sequence and amino acid sequence, respectively).

As used herein, “CD49e” is a protein generally called integrin α5. CD49e is also called ITGA5; CD49e; FNRA; or VLA5A. CD49e is also called fibronectin receptor α polypeptide. The α-chain 5 is cleaved in an extracellular domain by a modification after translation to produce a light chain and a heavy chain of disulfide bond, and bind to β1 to form a fibronectin receptor. The promoter for the expression of the molecule is positively regulated through a transcription factor Sp1 by an extracellular matrix component fibronectin in a corneal epithelial cell. Please refer to the document PMID: 10995740. CD49e is Entrez Gene ID: 3678, RefSeq (mRNA): NM_002205 (Hu), RefSeq (protein): NP_002196 (Hu). SEQ ID NOs: 11-12 are representative sequences thereof (nucleic acid sequence and amino acid sequence, respectively).

As used herein, “CD44” is a cell surface glycoprotein that forms cell-cell adhesion or cell-ECM (extra cellular matrix) adhesion. A CD44 molecule exhibits a structure rich in glycans, having multiple N-linked glycan sites, O-linked glycan sites, and chondroitin sulfate binding sites. Phosphorylated serine is present at a portion inside the cell next to a transmembrane section. CD44 is also a receptor of hyaluronic acid (HA) involved in cell movement (metastasis of cancer) and binding to actin through ankyrin. It is understood that CD44 can also interact with other ligands (e.g., osteopontin, collagen, and matrix metalloproteinase (MMP)). A human amniotic epithelial cell (hAEC) expresses CD9, CD44, CD73, and CD90, as well as negligible levels of CD31, CD34, CD45, and CD117. Please refer to the document PMID: 21166885. CD44 is also called CDW44; CSPG8; ECMR-III; HCELL; HUTCH-I; IN; LHR; MC56; MDU2; MDU3; MIC4; or Pgp1. CD44 is Entrez Gene ID: 960, RefSeq (mRNA): NM_000610, and RefSeq (protein): NP_000601. SEQ ID NOS: 13-14 are representative sequences thereof (nucleic acid sequence and amino acid sequence, respectively).

As used herein, “CD73” is an enzyme also called ecto-5′-nucleotidase, which converts AMP into adenosine. CD73 is also called NT5E; CD73; E5NT; NT; NT5; NTE; eN; or eNT. A protein encoded by this gene is a plasma membrane protein catalyzing a reaction that converts an extracellular nucleotide into a transmembrane nucleoside. A human amniotic epithelial cell (hAEC) expresses CD9, CD44, CD73, and CD90, as well as negligible levels of CD31, CD34, CD45, and CD117. An isolated corneal keratocyte exhibits a fibroblast-like shape and expresses CD13, CD29, CD44, CD56, CD73, CD90, CD105, and CD133, but is negative for HLA-DR, CD34, CD117, and CD45. These properties are similar to those of BM-MSC (bone marrow mesenchymal stem cells). It is understood that marginal bone marrow mesenchymal stem cells (L-MSC) are CD34⁻, CD34⁻, CD90⁺, CD73⁺, and CD105⁺. Please refer to the documents PMID: 21166885, PMID: 17464757 and PMID: 22587591. CD73 is Entrez Gene ID: 4907, RefSeq (mRNA): NM_001204813, and RefSeq (protein): NP_001191742. SEQ ID NOs: 15-16 are representative sequences thereof (nucleic acid sequence and amino acid sequence, respectively). For other CDS, information in, for example, http://hcdm.org/index.php/component/molecule/?Itemid=132 can be referred. Further, the following is known, and such information can be utilized for the implementation of the present invention. The content of such information is incorporated herein by reference as needed.

TABLE 1

| | Accession number of | Accession number of | |
|---|---|---|---|
| | human amino acid | human nucleic acid | |
| CD and explanation thereof | sequence | sequence | Entrez Gene ID: |
| HLA-A2 | — | — | — |
| (See Komlos L, Klein T. | | | |
| Korostishevsky M (2007). “HLA-A2 | | | |
| class I antigens in couples with | | | |
| recurrent spontaneous abortions”. | | | |
| Int. J. Immunogenet. 34 (4): 241-6 | | | |
| and the like) | | | |
| CD66a (CEACAM1) | NM_001024912 | NP_001020083 | Entrez 634 |
| CD66c (CEACAM6) | NM_002483 | NP_002474 | Entrez 4680 |
| CD66d (CEACAM3) | NM_001277163 | NP_001264092 | Entrez 1084 |
| CD66e (CEACAM5) | NM_004363 | NP_004354 | Entrez 1048 |
| CD59 (MAC-IP) complement | NM_000611 | NP_000602 | Entrez 966 |
| control protein is a cell surface | | | |
| glycoprotein which controls | | | |
| complement-mediated | | | |
| cytolysis. It is associated with | | | |
| lymphocyte signaling. Corneal | | | |
| cells are known to be resistant | | | |
| to complements. Relation to | | | |
| high level of CD59 expression in | | | |
| corneal epithelium is | | | |
| considered See document PMID: | | | |
| 16061345 (Sladowski D et al., | | | |
| Toxicol In Vitro2005 October: 19(7): | | | |
| 875-8) | | | |
| CD54 (ICAM-1) | NM_000201 | NP_000192 | Entrez 3383 |
| CD47 (IAP) | NM_001025079 | NP_001768 | Entrez 961 |
| EGF-R | NM_005228 | NM_005219 | Entrez 1956 |
| CD29 (ITGB1) integrin β1 | NM_002211 | NP_002202 | Entrez 3688 |
| (fibronectin receptor, β | | | |
| polypeptide, CD29 includes MDF2, | | | |
| MSK12). Required for maintaining | | | |
| the integrity of a corneal structure. | | | |
| See document PMID: | | | |
| 21873563 (Parapuram SK et al., | | | |
| Invest Ophthalmol Vis Sci. 2011 October | | | |
| 3:52(11): 7799-806) | | | |
| CD74 (HLADG) | NM_001025158 | NP_001020329 | Entrez 972 |
| CD165: a difference is observed in | — | — | Gene ID: 23449 |
| molecular phenotypes and | | | |
| differentiation potency for human | | | |
| stromal (mesenchymal) stem cells | | | |
| (MSC) from the bone marrow, fat | | | |
| tissue and skin. A microarray-based | | | |
| study has found 33 common MSC | | | |
| molecular signatures, including | | | |
| CD165, CD276, and CD82. | | | |
| See document PMID: | | | |
| 22529014(Al-Nbaheen M et al., | | | |
| Stem Cell Rev. 2013 February; | | | |
| 9(1): 32-43) | | | |
| CD221 | NM_000875 | N13_000866 | Entrez 3480 |
| CD49a (ITGA1) | NM_181501 | NP_852478 | Entrez 3672 |
| See | — | — | — |
| SSEA-4 (Stage Specific | | | |
| Embryonic Antigen-; Neurochem | | | |
| Res. 2011 September: 36(9): 1623-35 | | | |
| and the like | | | |
| CD 130 (gp130, IL6ST, IL6-beta) | NM_001190981 | NP_001177910 | Entrez 3572 |
| CD49f (ITGA6) | NM_000210 | NP_000201 | — |
| CD26 (DPP4) | NM_001935 | NP_001926 | Entrez 1803 |
| CD49b (ITGA2) | NM_002203 | NP_002194 | Entrez 3673 |
| CD13 (ANPEP) | NM_001150 | NP_001141 | Entrez 290 |
| CD99 (MIC2) | NM_001122898 | NP_001116370 | Entrez 4267 |
| CD105 (Endogrin) | NM_000118 | NP_000109 | Entrez 2022 |
| CD63 (LAMP-3): This molecule is | NM_001040034 | NP_001244318 | Entrez 967 |
| a member of the transmembrane | | | |
| 4 superfamily, belonging to the | | | |
| tetraspanin family. CD63 is a cell | | | |
| surface glycoprotein, complexed | | | |
| with integrins. CD63 may also | | | |
| function as a platelet activation | | | |
| marker. | | | |
| CD58 (LFA-3) | NM_001779 | — | Entrez 965 |
| CD201 (EPCR) | NM_006404 | NP_006395 | Entrez 10544 |
| CD56 (NCAM) | NM_000615 | NP_000606 | Entrez 4684 |
| CD55 (DAF) | NM_000574 | NP_000565 | Entrez 1604 |
| CD71 (TfR1) | NM_001128148 | NP_001121620 | Entrez 7037 |
| CD91 (LRP1) | NM_002332 | NP_002323 | Entrez 4035 |
| HLA-DQ, T-lymphocyte is activated | (HLA-DQA1 and HLA-DQB1) | | |
| by a corneal endothelial cell, and | | | |
| HLA-DP, -DQ, DR, and CD40 | | | |
| expression increases by | | | |
| pretreatment with γ interferon | | | |
| See document PMID: | | | |
| 18552515(Wang F., | | | |
| Ophthalmologica. | | | |
| 2008: 222(4): 272-6) | | | |
| Induction of class H antigens in | | | |
| human corneal epithelial cells is | | | |
| independently regulated by | | | |
| interferon γ for each antigen. DQ | | | |
| induction may depend on | | | |
| differentiation of cultured human | | | |
| corneal epithelial cells. | | | |
| See document PMID: | | | |
| 1639617(Iwata M, et at, Invest | | | |
| Ophthalmol Vis Sci. 1992 August: | | | |
| 33(9): 2714-21) | | | |
| .HLA-DQA1 | NM_002122 | NP_002113 | Entrez 3117 |
| .HLA-DQB1 | NM_001243961 | NP_001230890 | Entrez 3119 |
| CD164 | NM_001142401 | NP_001135873 | Entrez 8763 |
| CD49d | NM_000885 | NP_000876 | Entrez 3676 |
| CD49c (ITGA3) | NM_002204 | NP_002195 | Entrez 3675 |
| CD90 (Thy-1) | NM_006288 | NP_006279 | Entrez 7070 |
| MICA/B | (MICA (MHC class I chain-related gene A), | | |
| | MICB (MHC class I chain-related gene B)) | | |
| MICA: | NM_000247 | NP_000238 | Entrez 100507436 |
| MICB: | NM_005931 | NP_005922 | Entrez 4277 |
| CD46 | NM_002389 | NP_002380 | Entrez 4179 |
| CD140b (PDGFRB = | NM_002609 | NP_002600 | Entrez 5159 |
| Platelet-derived growth factor | | | |
| receptor, β polypeptide | | | |
| CD146 (MCAM | MUC18) | NM_006500 | Entrez 4162 |
| | | NP_006491 | |
| CD147 (Basigin (BSG), | NM_001728 | NP_001719 | Entrez 682 |
| EMMPRIN) | | | |
| CD81 | NM_004356 | NP_004347 | Entrez 975 |
| CD151 | NM_001039490 | NP_001034579 | Entrez 977 |
| CD200 (OX-2 membrane glycoprotein), | NM_001004196 | NP_001004196 | Entrez 4345 |
| may regulate bone marrow cell activity | | | |
| and transmits inhibition signals for the | | | |
| macrophage system in various tissues. | | | |
| An HSV infection results in infiltration | | | |
| into the cornea by CD200R(+) cells. | | | |
| Most of the cells are CD11 (+). See | | | |
| document PMID: 19070547 | | | |
| (Sarangi PP et al., Clin Immunol. 2009 | | | |
| April: 131(1): 31-40) | | | |
| CD10(Neprilysin, | NM_000902 | NP_000893 | Entrez 4311 |
| NEP, CALLA) | | | |

It is understood that each CD name refers to not only a protein (or a nucleic acid encoding the same) having the amino acid sequence described by the specific accession number (and SEQ ID NOs when specifically described herein), but also functionally active derivatives thereof, functionally active fragments thereof, homologs thereof, and mutants encoded by a nucleic acid that hybridizes to a nucleic acid encoding the protein under a highly or lowly stringent condition.

The same applies to all other proteins mentioned in the present invention. Thus, the predetermined names of proteins or nucleic acids not only refer to proteins or nucleic acids set forth in Sequence Listing, but also functionally active derivatives, functionally active fragments thereof, homologs thereof, and mutants encoded by a nucleic acid that hybridizes to a nucleic acid encoding the protein under a highly or lowly stringent condition, preferably under conditions discussed above. As used herein, a “derivative”, “analog of a constituent protein”, or “mutant” preferably includes a molecule comprising a region substantially homologous to a constituent protein, but not intended to be limited thereto. Such a molecule is at least 30%, 40%, 50%, 60%, 70%, 80%, 90%, 95% or 99% identical throughout the amino acid sequence of the same size or compared to a sequence aligned by a homology computer program known in the art in various embodiments. Alternatively, a nucleic acid encoding such a molecule can hybridize to a sequence encoding the constituent protein under a stringent condition, moderately stringent condition, or non-stringent condition. This is the outcome of modifying a naturally-occurring protein by an amino acid substitution, deletion and addition, respectively, and indicates that a derivative thereof exhibits the biological function of the naturally-occurring protein, although not necessarily to the same degree. For instance, the biological function of such a protein can be studied by a suitable and available in vitro assay described herein or known in the art. The discussion in the present invention mainly pertains to humans, but it is understood that the discussion is applicable to other species, such as other species within primates, or animal species in other genera. It is understood that mammals are within the scope of the present invention.

As used herein, “functionally active” refers to polypeptides of the present invention, i.e., polypeptides having a structural function, regulatory function or biochemical function of a protein such as biological activity according to an embodiment associated with the fragments or derivatives of the present invention.

In the present invention, a “fragment” of a molecule such as CD98 is a polypeptide comprising any region of the molecule such as CD98. As long as the fragment can be used for the objective of the present invention (e.g., detection, diagnosis, or the like), the fragment may not have the natural biological function of the molecule. An example of a fragment includes, but is not limited to, a fragment comprising an extracellular region of a molecule such as CD98.

A representative nucleotide sequence of a molecule such as CD98 may be:
(a) a polynucleotide having a base sequence set forth in SEQ ID NO: X or a fragment sequence thereof;
(b) a polynucleotide encoding a polypeptide consisting of the amino acid sequence set forth in SEQ ID NO: X+1 or a fragment thereof;
(c) a polynucleotide encoding a variant polypeptide having a mutation selected from the group consisting of substitution, addition and deletion in one or more amino acids in the amino acid sequence set forth in SEQ ID NO: X+1, or a fragment thereof, where the variant polypeptide has biological activity;
(d) a polynucleotide, which is a splice mutant or allelic mutant of the base sequence set forth in SEQ ID NO: X, or a fragment thereof;
(e) a polynucleotide encoding a species homolog of a polypeptide consisting of the amino acid sequence set forth in SEQ ID NO: X+1 or a fragment thereof;
(f) a polynucleotide encoding a polypeptide having biological activity and hybridizing to the polynucleotide according to any one of (a)-(e) under a stringent condition; or
(g) a polynucleotide encoding a polypeptide having biological activity and consisting of the polynucleotide according to any one of (a)-(e) or base sequence having at least 70% identity to a complement sequence thereof. Herein, biological activity typically refers to activity of a molecule such as CD98.

The amino acid sequence of a molecule such as CD98 may be:
(a) a polypeptide consisting of the amino acid sequence set forth in SEQ ID No.: X+1 or a fragment thereof;
(b) a polypeptide having one mutation selected from the group consisting of substitution, addition and deletion in one or more amino acids in the amino acid sequence set forth in SEQ ID NO: X+1 and having biological activity;
(c) a polypeptide encoded by a splicing mutant or an allele mutant of the base sequence set forth in SEQ ID NO: X+1;
(d) a polypeptide which is a species homolog of the amino acid sequence set forth in SEQ ID NO: X+1; or
(e) a polypeptide having an amino acid sequence having at least 70% identity to the polypeptide according to any one of (a)-(d), and having biological activity.

Herein, SEQ ID NO: X (X is an odd number from 1-15) indicates a nucleic acid sequence of a molecule (including a subunit) such as CD98. X=1 is a heavy subunit protein of CD98, X=3 is a light subunit protein of CD98, X=5 is CD166, X=7 is CD340, X=9 is CD9, X=11 is CD49e, X=13 is CD44, and X=15 is CD73. Biological activity typically refers to activity of a molecule such as CD98. Further, the nucleic acid sequences of SEQ ID NOs: 1-15 may comprise a sequence other than CDR (coding region). Such regions can also be used as a primer or a probe. In other embodiments, only CDR may be utilized as a nucleic acid region.

As used herein, “such as CD98” refers to any marker in the present invention. More specifically, markers expressed significantly more in normal cells than transformed cells include, but not limited to, CD166, HLA-A2, CD66a, CD66c, CD66d, CD66e, CD98, CD59, CD54, CD340, CD47, EGF-R, CD29, CD74, CD165, CD221, CD49a, SSEA-4, CD130, and CD49f. Markers expressed significantly more in transformed cells than in normal cells include, but not limited to, CD26, CD9, CD49b, CD49e, CD13, CD99, CD105, CD63, CD58, CD201, CD56, CD44, CD55, CD71, CD73, CD91, HLA-DQ, CD164, CD49d, CD49c, CD90, MICA/B, CD46, CD140b, CD146, CD147, CD81, CD151, CD200, and CD10.

In the context of the present invention, “substance that binds to a molecule such as CD98” or “molecular interaction molecule such as CD98” is a molecule or substance that binds at least transiently to a molecule such as CD98 and is preferably capable of indicating that the molecule or substance is bound (i.e., labelled or labelable state). A substance that binds to a molecule such as CD98 may be an inhibitor of the molecule such as CD98. Examples thereof include antibodies, antisense oligonucleotides, siRNA, low molecular weight molecules (LMW), binding peptides, aptamers, ribozymes, peptidomimetics and the like, as well as, for instance, binding proteins or binding peptides directed to a molecule such as CD98, especially those directed to an active site of the molecule such as CD98 and nucleic acids directed to a gene of the molecule such as CD98. A nucleic acid for a molecule such as CD98 refers to, for example, double stranded or single stranded DNA or RNA for inhibiting the expression of a gene of the molecule such as CD98 or activity of a molecule such as CD98 or a modified product or derivative thereof, including, without limitation, antisense nucleic acid, aptamers, siRNA (small interfering RNA) and ribozymes. As used herein, “binding protein” or “binding peptide” with respect to a molecule such as CD98 refers to a type of protein or peptide that binds to the molecule such as CD98, and includes, but is not limited to, polyclonal antibodies or monoclonal antibodies, antibody fragments and protein backbones directed to the molecule such as CD98.

As used herein, “protein”, “polypeptide”, “oligopeptide” and “peptide” are used herein to have the same meaning and refer to a polymer of amino acids with any length. The polymer may be straight, branched or cyclic. An amino acid may be a natural-occurring, non-naturally occurring or modified amino acid. The term may also encompass those assembled into a complex of multiple polypeptide chains. The term also encompasses naturally-occurring or artificially modified amino acid polymers. Examples of such a modification include disulfide bond formation, glycosylation, lipidation, acetylation, phosphorylation, and any other manipulation or modification (e.g., conjugation with a labeling component). The definition also encompasses, for example, polypeptides comprising one or more analogs of an amino acid (e.g., including non-naturally occurring amino acids and the like), peptide-like compounds (e.g., peptoids) and other known modifications in the art.

As used herein, “amino acid” may be naturally-occurring or non-naturally-occurring amino acids as long as the objective of the present invention is met.

As used herein, “polynucleotide”, “oligonucleotide” and “nucleic acid” are used herein to have the same meaning, and refer to a polymer of nucleotides with any length. The term also encompasses “oligonucleotide derivative” and “polynucleotide derivative”. “Oligonucleotide derivative” and “polynucleotide derivative” refer to an oligonucleotide or polynucleotide that comprises a nucleotide derivative or has a linkage between nucleotides which is different from normal. The terms are used interchangeably. Specific examples of such an oligonucleotide include 2′-O-methyl-ribonucleotide, oligonucleotide derivatives having a phosphodiester linkage in an oligonucleotide converted to a phosphorothioate linkage, oligonucleotide derivatives having a phosphodiester linkage in an oligonucleotide converted to an N3′-P5′ phosphoramidate linkage, oligonucleotide derivatives having ribose and phosphodiester linkage in an oligonucleotide converted to a peptide nucleic acid linkage, oligonucleotide derivatives having uracil in an oligonucleotide replaced with C-5 propinyluracil, oligonucleotide derivatives having uracil in an oligonucleotide replaced with C-5 thiazoluracil, oligonucleotide derivatives having cytosine in an oligonucleotide replaced with C-5 propinylcytosine, oligonucleotide derivatives having cytosine in an oligonucleotide replaced with phenoxazine-modified cytosine, oligonucleotide derivatives having ribose in DNA replaced with 2′-O-propylribose, oligonucleotide derivatives having ribose in an oligonucleotide replaced with 2′-methoxyethoxyribose and the like. Unless noted otherwise, specific nucleic acid sequences are also intended to encompass conservatively modified variants (e.g., degenerate codon substitute) and complement sequences as in the expressly shown sequences. Specifically, degenerate codon substitutes can be achieved by preparing a sequence with the third position of one or more selected (or all) codons substituted with a mixed base and/or deoxyinosine residue (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). As used herein, “nucleic acid” is used interchangeably with a gene, CDNA, mRNA, an oligonucleotide, and polynucleotide. As used herein, a “nucleotide” may be a naturally-occurring or non-naturally occurring amino acid.

As used herein, “gene” refers to an agent defining a genetic trait. A gene is generally arranged in a certain order on a chromosome. A gene defining the primary structure of a protein is referred to as a structural gene and a gene determining the expression thereof is referred to as a regulator gene. As used herein, “gene” may refer to “polynucleotide”, “oligonucleotide” and “nucleic acid”. A “gene product” is a substance produced based on a gene and refers to a protein, mRNA or the like.

As used herein, “homology” of genes refers to the level of identity of two or more genetic sequences with one another. In general, having “homology” refers to having a high level of identity or similarity. Thus, two genes with high homology have higher identity or similarity of sequences. It is possible to find whether two types of genes have homology by direct comparison of sequences or, in the case for nucleic acids, by a hybridization method under a stringent condition. When two genetic sequences are directly compared, the genes are homologous when DNA sequences are representatively at least 50% identical, preferably at least 70% identical, and more preferably at least 80%, 90%, 95%, 96%, 97%, 98%, or 99% identical between the genetic sequences. Thus, as used herein, “homolog” or “homologous gene product” refers to a protein in another species, preferably mammal, exerting the same biological function as a protein constituent of a complex which will be further described herein. Such a homolog is also called “ortholog gene product”. An algorithm for detecting orthologous gene pair from humans and mammals or another species uses the entire genome of these organisms. First, an expected complete Smith-Waterman alignment of a protein is used to a collect a pairwise best hit. To further improve the reliability, a pairwise best hit comprising Drosophila melanogaster and C. elegans proteins may be used to form a cluster of pairs. Such analysis is provided in, for example, Nature, 2001, 409:860-921. Based on sequence homology of genes encoding the proteins provided herein with respect to genes of other species, it is also possible to isolate a homolog of the proteins described herein by applying a conventional technique to clone each gene and allowing a protein from such genes to be expressed or by isolating a similar complex to isolate a protein of another species in accordance with the method provided herein or in accordance with other suitable methods well known in the art.

Amino acids may be mentioned herein by either the common, known three letter symbol thereof or by a one character symbol recommended by the IUPAC-IUB Biochemical Nomenclature Commission. Nucleotides may similarly be mentioned by a commonly recognized one character code. Comparison of similarity, identity and homology of an amino acid sequence and a base sequence is calculated herein by using a default parameter using BLAST, which is a sequence analysis tool. For example, identity can be searched by using BLAST 2.2.9 (published on May 12, 2004) of NCBI. Herein, values for identity generally refer to a value obtained by alignment under the default condition using the above-described BLAST. However, when a higher value is obtained by changing a parameter, the highest value is considered the value of identity. When identity is evaluated in multiple regions, the highest value thereamong is considered the value of identity. Similarity is a value by taking into consideration similar amino acid in addition to identity into the calculation.

As used herein, polynucleotide which “hybridizes under a stringent condition” refers to commonly used, well-known conditions in the art. Such a polynucleotide can be obtained by using a method such as colony hybridization, plaque hybridization, or southern blot hybridization while using a polynucleotide selected from among the polynucleotides of the present inventions as a probe. Specifically, the above-described polynucleotide refers to a polynucleotide that can be identified by using a filter with immobilized DNA from a colony or plaque and performing hybridization at 65° C. in the presence of 0.7-1.0 M NaCl and then using an SSC (saline-sodium citrate) solution with 0.1-2 times concentration (composition of an SSC solution with 1 time concentration is 150 mM sodium chloride and 15 mM sodium citrate) and washing the filter under the condition of 65° C. Hybridization can be performed in accordance with the method described in an experimental manual such as Molecular Cloning 2nd ed., Current Protocols in Molecular Biology, Supplement 1-38, DNA Cloning 1: Core Techniques, A Practical Approach, Second Edition, Oxford University Press (1995) or the like. In this regard, sequences comprising only A sequence or T sequence are preferably excluded from the sequence that hybridizes under a stringent condition. Thus, the polypeptides used in the present invention (e.g., transthyretin and the like) also encompass polypeptides encoded by a nucleic acid molecule that hybridizes under stringent condition to a nucleic acid molecule encoding a polypeptide especially described in the present invention. Such low stringency conditions include hybridization for 18-20 hours at 40° C. in a buffer solution comprising 35% formamide, 5×SSC, 50 mM Tris-HCl (pH 7.5), 5 mM EDTA, 0.02% polyvinylpyrrolidone (PVP), 0.02% BSA, 100 μg/ml denatured salmon sperm DNA, and 10% (w/v) dextran sulfate, washing 1-5 hours at 55° C. in a buffer consisting of 2×SSC, 25 mM Tris-HCl (pH 7.4), 5 mM EDTA and 0.1% SDS, and washing for 1.5 hours at 60° C. in a buffer consisting of 2×SSC, 25 mM Tris-HCl (pH 7.4), 5 mM EDTA, and 0.1% SDS.

As used herein, a “purified” substance or biological agent (e.g., nucleic acid or protein) refers to a substance or a biological agent from which at least a part of an agent naturally associated with the biological agent has been removed. Thus, the purity of a biological agent in a purified biological agent is generally higher than the purity in the normal state of the biological agent (i.e., concentrated). The term “purified” as used herein refers to the presence of preferably at least 75% by weight, more preferably at least 85% by weight, still more preferably at least 95% by weight, and most preferably at least 98% by weight of the same type of a biological agent. A substance used in the present invention is preferably a “purified” substance.

As used herein, a “corresponding” amino acid or nucleic acid refers to an amino acid or a nucleotide which has or is expected to have, in a certain polypeptide molecule or polynucleotide molecule, similar action as a predetermined amino acid or nucleotide in a benchmark polypeptide or a polynucleotide, and, particularly in the case of enzyme molecules, refers to an amino acid which is present at a similar position in an active site and makes a similar contribution to catalytic activity. For example, for an antisense molecule, it can be a similar part in an ortholog corresponding to a specified part of the antisense molecule. A corresponding amino acid can be a specified amino acid subjected to, for example, cysteination, glutathionylation, S—S bond formation, oxidation (e.g., oxidation of methionine side chain), formylation, acetylation, phosphorylation, glycosylation, myristylation or the like. Alternatively, a corresponding amino acid can be an amino acid responsible for dimerization. Such a “corresponding” amino acid or nucleic acid may be a region or a domain over a certain range. Thus, it is referred herein as a “corresponding” region or domain in such a case.

As used herein, a “corresponding” gene (e.g., polynucleotide sequence or molecule) refers to a gene (e.g., polynucleotide sequence or molecule) of a certain species which has or is expected to have similar action as that of a predetermined gene in a benchmark species. When there is a plurality of genes having such action, the corresponding gene refers to a gene having the same evolutionary origin. Thus, a corresponding molecule such as CD98 can be found in humans for each of molecules such as CD98 of mice and rats. Such a corresponding gene can be identified by using a technique that is well known in the art. For example, a corresponding gene in a certain animal (e.g., mouse, or rat) can be found by searching a sequence database of the animal using the sequence of SEQ ID NO: 1 or the like as a query sequence as a benchmark gene of a corresponding gene (e.g., of a human).

As used herein, a “fragment” refers to a polypeptide or a polynucleotide having a sequence length of 1 to n−1, relative to a full length polypeptide or polynucleotide (length: n). The length of the fragment can be appropriately changed depending on the objective thereof. Examples of the lower limit of the length for a polypeptide include 3, 4, 5, 6, 7, 8, 9, 10, 15, 20, 25, 30, 40, 50 and more amino acids. A length represented by an integer which is not specifically listed herein (e.g., 11 or the like) can also be appropriate as the lower limit. For a polynucleotide, examples of the lower limit include 5, 6, 7, 8, 9, 10, 15, 20, 25, 30, 40, 50, 75, 100 and more nucleotides. A length represented by an integer which is not specifically listed herein (e.g., 11 or the like) can also be appropriate as the lower limit. For such a fragment as used herein, it is understood, for example, that when a full length polypeptide or polynucleotide functions as a marker, the fragment itself is also within the scope of the present invention as long as it functions as a marker.

According to the present invention, the term “activity” as used herein refers to the function of a molecule in a broadest sense. The activity is not intended to be particularly limited, but generally includes a biological function, a biochemical function, a physical function or a chemical function of a molecule. The activity includes, for example, an enzyme activity, an ability to interact with other molecules, an ability to activate, promote, stabilize, inhibit, suppress, or destabilize the function of other molecules, stability, and an ability to be localized at a specific position in a cell. When applicable, this term also relates to the function of a protein complex in a broadest sense.

As used herein, “biological function”, when referring to a certain gene or a nucleic acid molecule or a polypeptide related thereto, refers to a specific function that the gene, the nucleic acid molecule or the polypeptide may have in a living body. Examples of such a function include, but are not limited to, production of a specific antibody, enzyme activity, impartation of resistance and the like. In the present invention, examples of this function include, but are not limited to, a function of CD98 or the like of recognizing a ligand. Documents cited by an Entrez number, accession number or the like listed in the aforementioned Tables can be referred for such biological activity. Such documents or the like are incorporated herein by reference. As used herein, biological function can be exerted by “biological activity”. As used herein, “biological activity” refers to the activity a certain agent (e.g., polynucleotide, protein or the like) may have in a living body. Biological activity encompasses an activity of exerting a variety of functions (e.g., transcription promoting activity), and also encompasses, for example, an activity that activates or inactivates another molecule by an interaction with a certain molecule. When two agents interact, biological activity thereof is understood to be binding between two molecules and a biological change induced thereby. For example, two molecules are considered to be bound together if, when one molecule is precipitated using an antibody, the other molecule co-precipitates. Observation of such co-precipitation is one example of a determination procedure. For example, when a certain agent is an enzyme, the biological activity thereof encompasses enzyme activity. In another example, when a certain agent is a ligand, binding to a receptor corresponding to the ligand is encompassed. Such biological activity can be measured by a technique that is well known in the art. Thus, “activity” refers to various measurable indicators, which indicate or reveal binding (either direct or indirect) or affect a response (i.e., having a measurable effect in response to some exposure of stimulus). Examples thereof includes affinity of a compound that directly binds to the polypeptide or polynucleotide of the present invention, the amount of proteins upstream or downstream after some stimulations or events, or the measure of other similar functions.

As used herein, “expression” of a gene, a polynucleotide, a polypeptide or the like refers to the gene or the like being subjected to a certain action in vivo to be converted into another form. Preferably, expression refers a gene, a polynucleotide or the like being transcribed and translated into a form of a polypeptide. However, transcription to make mRNA can also be one embodiment of expression. More preferably, such a form of a polypeptide can be a form which has undergone processing after translation (derivative as is referred to herein). For example, the expression level of a molecule such as CD98 can be determined by any method. Specifically, the expression level of a molecule such as CD98 can be found by evaluating the amount of mRNA of a molecule such as CD98, the amount of protein of a molecule such as CD98, and the biological activity of protein of a molecule such as CD98. The amount of mRNA or a protein of a molecule such as CD98 can be determined by the method described herein.

As used herein, it is understood that a “molecule such as CD98 or functional equivalent thereof” or a “group consisting of molecule such as CD98 and functional equivalents thereof” refers to, in addition to the molecule such as CD98 itself, mutants or variants of the molecule such as CD98 (e.g., amino acid sequence variant and the like) which have an action of controlling differentiation and/or promoting proliferation of ocular cells or the like or function as a marker described herein, as well as those that can be changed into the molecule such as CD98 itself or a mutant or a variant thereof (e.g., including nucleic acids encoding the molecule such as CD98 itself or a mutant or a variant of the molecule such as CD98, vectors, cells and the like comprising the nucleic acid) at the time of action. In the present invention, it is understood, even when not specifically mentioned, that a functional equivalent of a molecule such as CD98 can be used similarly to the molecule such as CD98.

A functional equivalent of a molecule such as CD98 used in the present invention can be found by searching a database or the like. As used herein, “search” refers to finding another nucleic acid base sequence having a specific function and/or property by utilizing a certain nucleic acid base sequence with electronic, biological, or other methods. Electronic search includes, but is not limited to, BLAST (Altschul et al., J. Mol. Biol. 215:403-410 (1990)), FASTA (Pearson & Lipman, Proc. Natl. Acad. Sci., USA 85:2444-2448 (1988)), Smith and Waterman method (Smith and Waterman, J. Mol. Biol. 147:195-197 (1981)), Needleman and Wunsch method (Needleman and Wunsch, J. Mol. Biol. 48:443-453 (1970)) and the like. Biological search includes, but is not limited to, stringent hybridization, microarray in which genomic DNA is applied on a nylon membrane, glass plate or the like (microarray assay), PCR, in situ hybridization and the like. Herein, genes used in the present invention are intended to include corresponding genes identified by such electronic or biological search.

An amino acid sequence with an insertion, substitution, deletion, or addition to one or both ends thereof of one or more amino acids can be used as a functional equivalent of the present invention. As used herein, “amino acid sequence with an insertion, substitution, deletion, or addition to one or both ends thereof of one or more amino acids” means that a sequence is modified by a well-known technical method such as site-specific mutagenesis, or by substitution of a plurality of amino acids to the extent that may occur naturally by natural mutations.

A modified amino acid sequence of a molecule such as CD98 can be a sequence with, for example, insertion, substitution, deletion, or addition to one or both ends, of 1 to 30, preferably 1 to 20, more preferably 1 to 9, still more preferably 1 to 5, and especially preferably 1 to 2 amino acids. A modified amino acid sequence may be an amino acid sequence of a molecule such as CD98 preferably having one or more (preferably, 1 or a few, or 1, 2, 3 or 4) conservative substitutions. Herein, “conservative substitution” refers to substitution of one or more amino acid residues with other chemically similar amino acid residues so that the function of a protein is not substantially modified. Examples thereof include substitution of a certain hydrophobic residue with another hydrophobic residue, substitution of a certain polar residue with another polar residue having the same charge, and the like. A functionally similar amino acid which can be subjected to such substitution is known in the art for every amino acid. Specific examples thereof as a non-polar (hydrophobic) amino acid include alanine, valine, isoleucine, leucine, proline, tryptophan, phenylalanine, methionine and the like. Examples thereof as a polar (neutral) amino acid include glycine, serine, threonine, tyrosine, glutamine, asparagine, cysteine and the like. Examples thereof as a (basic) amino acid having a positive charge include arginine, histidine, lysine and the like. Further, examples thereof as an (acidic) amino acid having a negative charge include aspartic acid, glutamic acid and the like.

As used herein, a “marker (substance, protein, or gene (nucleic acid))” refers to a substance which serves as a indicator for tracking whether a cell is in a certain state (e.g., the level or presence of a normal cell state, a transformed state, a disease state, a disorder state, a proliferation ability, or a differentiated state), or whether there is risk thereof. Examples of such a marker include genes (nucleic acid=DNA level), gene products (mRNA, protein and the like), metabolites, enzymes and the like. In the present invention, detection, diagnosis, preliminary detection, prediction or advance diagnosis of a certain state (e.g., a disease such as differentiation disorder) can be realized using an agent or means specific to a marker associated with the state, or a composition, a kit, a system or the like comprising them. As used herein, “gene product” refers to mRNA or a protein encoded by a gene. It was found herein that a gene product which has not been shown to be associated with an ocular cell (i.e. molecule such as CD98, etc.) can be used as an indicator of whether an ocular cell, especially a corneal endothelial cell, is normal (whether the cell is transformed).

As used herein, “nerve cell” is used in a broad sense, referring to any cell included in an organ of the nervous system. It is understood in particular that cells derived from a neural crest cell (e.g., corneal endothelial cell and the like) are also encompassed.

As used herein, “ocular cell” is used in a broad sense, referring to any cell in an eye. Any cell in the eyelid, sclera, cornea, uvea, crystalline lens, vitreous body, retina, and optic nerve is encompassed thereby.

As used herein, “corneal endothelial cell” is used in the general sense used in the art. The cornea is one of the lamellar tissues constituting an eye. A cornea is transparent and is positioned at a part closest to the external environment. In humans, it is understood that the cornea is composed of five layers in order of corneal epithelium, Bowman's membrane (external boundary), Lamina propria, Descemet's membrane (internal boundary), and corneal endothelium from the outside (body surface). Unless specifically noted otherwise, parts other than epithelium and endothelium may be collectively called “corneal stroma”, which is also called as such herein.

As used herein, “corneal tissue” is used in the general sense, referring to the tissue itself constituting the cornea. When referring to a corneal tissue, it may include all constituents of a cornea, i.e., corneal epithelium, Bowman's membrane (external boundary), Lamina propria, Descemet's membrane (internal boundary), and corneal endothelium (for humans; for other animals, all corresponding sections thereof), lack a part, or include another tissue (sclera) in addition to the cornea, which may be particularly called sclerocornea. Thus, a sclerocornea or a sclerocornea fragment is recognized as one embodiment of a corneal tissue.

As used herein, “normal corneal endothelial cell marker” refers to a marker, which is expressed in a normal corneal endothelial cell and has significantly reduced amount of expression when transformed. Threshold values of expression in normal cells include, but are not limited to, about 1.01 times or more, about 1.02 times or more, about 1.03 times or more, about 1.04 times or more, about 1.05 times or more, about 1.1 times or more, about 1.2 times or more, about 1.3 times or more, about 1.4 times or more, about 1.5 times or more, about 1.6 times or more, about 1.7 times or more, about 1.8 times or more, about 1.9 times or more, about 2 times or more, about 2.5 times or more, about 3 times or more, about 3.5 times or more, about 4 times or more, about 4.5 times or more, about 5 times or more, about 6 times or more, about 7 times or more, about 8 times or more, about 9 times or more, about 10 times or more than the value in transformed cell and the like.

As used herein, “transformed corneal endothelial cell marker” refers to a marker, which is expressed in transformed corneal endothelial cell, but significantly less in normal cells. Threshold values of expression in transformed cells include, but are not limited to, about 1.01 times or more, about 1.02 times or more, about 1.03 times or more, about 1.04 times or more, about 1.05 times or more, about 1.1 times or more, about 1.2 times or more, about 1.3 times or more, about 1.4 times or more, about 1.5 times or more, about 1.6 times or more, about 1.7 times or more, about 1.8 times or more, about 1.9 times or more, about 2 times or more, about 2.5 times or more, about 3 times or more, about 3.5 times or more, about 4 times or more, about 4.5 times or more, about 5 times or more, about 6 times or more, about 7 times or more, about 8 times or more, about 9 times or more, about 10 times or more than the value in normal cells.

As used herein, “transform” refers to a characteristic of a cell changing to a non-normal state and includes to mean a normal animal cell undergoing unrestrained cell division, i.e., cancer formation, or especially dynamic metaplasia (dedifferentiation to be a stem cell or changes beyond the realm of basic form of tissue).

Whether a corneal endothelial cell is cultured normally or not can be determined herein by confirming if a corneal endothelial cell maintains at least one characteristic such as its inherent function (also referred to as “normal function” herein, and such a cell is also referred to as a “normal cell”). Examples of such a function include, but not limited to, adaptability to corneal transplantation, ZO-1, Na+/K+-ATPase and the like (Matsubara M, Tanishima T: Wound-healing of the corneal endothelium in the monkey: a morphometric study, Jpn J Ophthalmol 1982, 26:264-273; Matsubara M, Tanishima T: Wound-healing of corneal endothelium in monkey: an autoradiographic study, Jpn J Ophthalmol 1983, 27:444-450; Van Horn D L, Hyndiuk R A: Endothelial wound repair in primate cornea, Exp Eye Res 1975, 21:113-124 and Van Horn D L, Sendele D D, Seideman S, Buco P J: Regenerative capacity of the corneal endothelium in rabbit and cat, Invest Ophthalmol Vis Sci 1977, 16:597-613). That is, it is understood that the “normal function” may be an indicator indicating a function necessary for realizing corneal transplantation or sufficiency for realizing corneal transplantation. For example, a method of determining normalization can be performed by observing a change in expression using a functional protein in a corneal endothelial cell such as ZO-1 and Na+/K+-ATPase as an indicator, or investigating whether the cell is engrafted and functions by transplantation into a monkey or the like. A determination method by transplantation can be carried out as follows. That is, corneal endothelium is cultured on a carrier such as type I collagen, amnion, corneal stroma or the like to make a cultured corneal endothelium sheet. Under general anesthesia, 1.5 mm incision is made on the limbus corneae of a cynomolgus monkey, a silicon surgical tool is inserted into the anterior chamber, and a corneal endothelial cell is mechanically curetted to make a bullous keratopathy model. Subsequently, a 5 to 6 mm incision is made on the limbus corneae, the cultured corneal endothelium sheet is inserted into the anterior chamber, and the anterior chamber is replaced with air to make the sheet adhere to the corneal endothelium surface. The therapeutic effect of transplantation of the cultured corneal endothelium sheet on bullous keratopathy can be evaluated by cornea transparency with a slit lamp microscope. Further, a therapeutic effect on bullous keratopathy can be similarly evaluated by corneal transparency with a slit-lamp microscope by injecting cells as a suspension with a cell adhesion promotor such as a Rho kinase inhibitor into the anterior chamber without using a carrier. Thus, non-normal, i.e., transformed cells are not assessed as normal cells in this evaluation. Transformed cells have lost or weakened pumping and barrier functions, which are functions of a corneal endothelial cell. Thus, transformed cells are evaluated in categories such as non-transparent cornea, thick cornea, low corneal endothelial cell density and the like.

In the present invention, “Rho kinase” refers to serine/threonine kinase which is activated with activation of Rho. Examples thereof include ROKα (ROCK-II: Leung, T. et al., J. Biol. Chem., 270, 29051-29054, 1995), p160ROCK (ROKβ, ROCK-I: Ishizaki, T. et al., The EMBO J., 15 (8), 1885-1893, 1996) and other proteins having serine/threonine kinase activity.

Examples of Rho kinase inhibitors include compounds disclosed in the following documents: U.S. Pat. No. 4,678,783, Japanese Patent No. 3421217, International Publication No. WO 95/28387, International Publication No. WO 99/20620, International Publication No. WO 99/61403, International Publication No. WO 02/076976, International Publication No. WO 02/076977, International Publication No. WO 2002/083175, International Publication No. WO 02/100833, International Publication No. WO 03/059913, International Publication No. WO 03/062227, International Publication No. WO 2004/009555, International Publication No. WO 2004/022541, International Publication No. WO 2004/108724, International Publication No. WO 2005/003101, International Publication No. WO 2005/039564, International Publication No. WO 2005/034866, International Publication No. WO 2005/037197, International Publication No. WO 2005/037198, International Publication No. WO 2005/035501, International Publication No. WO 2005/035503, International Publication No. WO 2005/035506, International Publication No. WO 2005/080394, International Publication No. WO 2005/103050, International Publication No. WO 2006/057270, International Publication No. WO 2007/026664 and the like. Such compounds can be manufactured by the methods described in the respective documents where the compounds are disclosed. The specific examples thereof include 1-(5-isoquinolinesulfonyl) homopiperazine or a salt thereof (e.g., fasudil (1-(5-isoquinolinesulfonyl) homopiperazine)), (+)-trans-4-(1-aminoethyl)-1-(4-pyridylcarbamoyl) cyclohexane ((R)-(+)-trans-(4-pyridyl)-4-(1-aminoethyl)-cyclohexanecarboxamide) or a salt thereof (e.g., Y-27632 ((R)-(+)-trans-(4-pyridyl)-4-(1-aminoethyl)-cyclohexanecarboxamide dehydrochloride monohydrate) and the like) and the like. For these compounds, a commercially available product (Wako Pure Chemical Industries, Ltd, Asahi Kasei Pharma Corporation and the like) can also be preferably used.

Since (+)-trans-4-(1-aminoethyl)-1-(4-pyridylcarbamoyl) cyclohexane, 1-(5-isoquinolinesulfonyl) homopiperazine, pharmaceutically acceptable salts thereof and the like are particularly excellent for promoting adhesion of corneal endothelial cells, they are preferably used.] pharmaceutically acceptable acid addition salt is preferred as a salt of the compounds. Examples of such an acid include hydrochloric acid, hydrobromic acid, sulfuric acid and other inorganic acid, methanesulfonic acid, fumaric acid, maleic acid, mandelic acid, citric acid, tartaric acid, salicylic acid and other organic acids and the like. (+)-trans-4-(1-aminoethyl)-1-(4-pyridylcarbamoyl) cyclohexane ((R)-(+)-trans-(4-pyridyl)-4-(1-aminoethyl)-cyclohexanecarboxamide)·dihydrochloride (which may also be monohydrate) and 1-(5-isoquinolinesulfonyl) homopiperazine hydrochloride are more preferable.

As used herein, “specimen” refers to a subject which is to be subjected to diagnosis, detection or the like in the present invention (e.g., an organism such as a human, an organ (eye) or a cell which has been taken out from an organism or the like).

As used herein, a “sample” refers to any substance obtained from a specimen or the like, and includes, for example, a cell of an eye. Those skilled in the art can appropriately select a preferable sample based on the descriptions herein.

As used herein, an “agent” is used in a broad sense, and may be any substance or other elements (e.g., energy such as light, radiation, heat, and electricity) as long as the intended objective can be attained. Examples of such a substance include, but are not limited to, proteins, polypeptides, oligopeptides, peptides, polynucleotides, oligonucleotides, nucleotides, nucleic acids (e.g., including DNA such as cDNA and genomic DNA, and RNA such as mRNA), polysaccharides, oligosaccharides, fats, organic small molecules (e.g., hormones, ligands, information transmitting substances, organic small molecules, molecules synthesized by combinatorial chemistry, small molecules which can be utilized as a medicine (e.g., a low molecular weight ligand) and the like), and composite molecule thereof. Representative examples of an agent specific to a polynucleotide include, but are not limited to, a polynucleotide having complementarity with certain sequence homology (e.g., 70% or more sequence identity) relative to the sequence of the polynucleotide, a polypeptide such as a transcription factor binding to a promoter region. Representative examples of an agent specific to a polypeptide include, but are not limited to, an antibody specifically directed to the polypeptide or a derivative or an analog thereof (e.g., single-stranded antibody), a specific ligand or receptor when the polypeptide is a receptor or a ligand, and a substrate when the polypeptide is an enzyme.

As used herein, a “detection agent” in a broad sense refers to any agent capable of detecting a subject of interest (e.g., normal cells (e.g., normal corneal endothelial cell) or transformed cell (e.g., transformed corneal endothelial cell)).

As used herein, an “agent for diagnosis” in a broad sense refers to any agent with which a condition of interest (e.g., a disease or the like) can be diagnosed.

The detection agent of the present invention may be a complex or a composite molecule in which another substance (e.g., label or the like) is bound to a portion which allows detection (e.g., antibody or the like). As used herein, a “complex” or a “composite molecule” refers to any construct comprising two or more parts. For example, when one of the parts is a polypeptide, the other part may be a polypeptide or another substance (e.g., a sugar, a lipid, a nucleic acid, a different hydrocarbon or the like). As used herein, two or more parts constituting the complex may be bound by a covalent bond or another bond or linkage (e.g., a hydrogen bond, an ionic bond, hydrophobic interaction, or Van der Waals force). When the two or more parts are each a polypeptide, this can also be called a chimeric polypeptide. Thus, as used herein, a “complex” encompasses molecules obtained by connecting a plurality of kinds of molecules such as a polypeptide, a polynucleotide, a lipid, a sugar, and a small molecule.

As used herein, “interaction”, when referring to two substances, refers to a force (e.g., intermolecular force (Van der Waals force), a hydrogen bond, hydrophobic interaction or the like) being exerted between a substance and another substance. Generally, the two interacting substances are in an associated or a bound state.

The term “bond” or “linkage” or “binding” as used herein refers to physical interaction or chemical interaction between two substances or between combinations thereof. The bond, the linkage and the binding include an ionic bond, a non-ionic bond, a hydrogen bond, a Van der Waals bond, hydrophobic interaction, and the like. Physical interaction (bond) can be direct or indirect, where indirect bond is formed through or due to the effect of another protein or compound. A direct bond is not formed through or due to the effect of another protein or compound, referring to interaction accompanying substantially no other chemical intermediate. The level of expression of the marker of the present invention or the like can be measured by measuring the bond or interaction.

Thus, as used herein, an “agent” (or a detection agent or the like) which “specifically” interacts with (or binds to) a biological agent such as a polynucleotide or a polypeptide includes an agent whose affinity to the biological agent such as a polynucleotide or a polypeptide is representatively equal to or higher than, preferably significantly (e.g., statistically significantly) higher than the affinity to other irrelevant polynucleotide or polypeptide (particularly, those with less than 30% identity). Such affinity can be measured, for example, by a hybridization assay, a binding assay or the like.

As used herein, a first substance or agent “specifically” interacting with (or binding to) a second substance or agent refers to a first substance or agent interacting with (or binding to) a second substance or agent with higher affinity than that to a substance or agent other than the second substance or agent (particularly another substance or agent that is present in a sample containing the second substance or agent). Examples of interaction (or bond) specific to a substance or an agent include, but are not limited to, a ligand-receptor reaction, hybridization in nucleic acids, an antigen-antibody reaction in proteins, an enzyme-substrate reaction, and when both a nucleic acid and a protein are involved, a reaction between a transcription factor and a binding site of the transcription factor and the like, protein-lipid interaction, nucleic acid-lipid interaction and the like. Thus, when both of the substances or agents are nucleic acids, a first substance or agent “specifically interacting” with a second substance or agent encompasses the first substance or agent having complementarity to at least a part of the second substance or agent. For example, when both of the substances or agents are proteins, examples of “specific” interaction (or bond) of a first substance or agent with a second substance or agent includes, but are not limited to, interaction by an antigen-antibody reaction, interaction by a receptor-ligand reaction, enzyme-substrate interaction. When two kinds of substances or agents include a protein and a nucleic acid, “specific” interaction (or bond) of a first substance or agent with a second substance or agent encompasses interaction (or bond) between a transcription factor and a binding region of a nucleic acid molecule which is a subject of the transcription factor.

As used herein, “detection” or “quantitation” of polynucleotide or polypeptide expression can be attained, for example, by using an appropriate method including mRNA measurement and an immunological measuring method, which includes binding or interaction with a marker detection agent. Examples of a molecular biological measuring method include a Northern blotting method, a dot blotting method, a PCR method and the like. Examples of an immunological measuring method include, as a method, an ELISA method using a microtiter plate, an RIA method, a fluorescent antibody method, a luminescence immunoassay (LIA), an immunoprecipitation method (IP), a single radical immuno-diffusion method (SRID), turbidimetric immunoassay (TIA), a Western blotting method, and an immunohistological staining method. Further, examples of a quantitation method include, but are not limited to, an ELISA method and an RIA method. Detection or quantitation can also be performed by a genetic analysis method using an array (e.g., DNA array or protein array). The DNA array is extensively reviewed in (Cell Technology, separate volume, “DNA Microarray and Advanced PCR method”, edited by Shujunsha Co., Ltd.). A protein array is described in detail in Nat Genet. 2002 December; 32 Suppl: 526-532. Examples of a method for analyzing gene expression include, but are not limited to, RT-PCR, a RACE method, a SSCP method, an immunoprecipitation method, a two-hybrid system, in vitro translation and the like in addition to the aforementioned methods. Such additional analysis methods are described, for example, in Genome Analysis Experimental Method, Nakamura Yusuke Lab. Manual, edited by Yusuke Nakamura, Yodosha Co., Ltd. (2002) and the like. The entirety descriptions in the document are incorporated herein by reference.

As used herein, an “expression amount” refers to an amount of expression of a polypeptide, mRNA, or the like in a cell, tissue or the like of interest. Examples of such an expression amount include an expression amount at the protein level of the present polypeptide evaluated by any appropriate method including an immunological measuring method such as an immunohistological staining method, an ELISA method, an RIA method, a fluorescent antibody method, and a Western blotting method using the antibody of the present invention, and an expression amount at the mRNA level of a polypeptide used in the present invention which is evaluated by any appropriate method including a molecular biological measuring method such as a PCR method, a Northern blotting method, and a dot blotting method. “Change in an expression amount” refers to an increase or decrease in an expression amount at the protein level or the mRNA level of a polypeptide used in the present invention, which is evaluated by any appropriate method including the immunological measuring method and the molecular biological measuring method described above. By measuring an expression amount of a certain marker, a variety of detections or diagnoses based on the marker can be performed.

As used herein, “decrease”, “suppression” or a synonym thereof of the activity, an expression product (e.g., a protein or a transcription product (such as RNA) or the like) refers to a decrease in an amount, quality or the effect of a specific activity, transcription product or protein, or an activity that decreases them.

As used herein, “increase”, “activation” or a synonym thereof of the activity or an expression product (e.g., a protein, a transcription product (such as RNA) or the like) refers to an increase in an amount, quality or the effect of a specific activity, transcription product or protein, or an activity that increases them.

Thus, it is understood that an agent which performs differentiation modulation of an ocular cell can be detected or screened using the modulation ability such as decrease, suppression, increase or activation of the marker of the present invention as an indicator.

As used herein, an “antibody” includes, in a broad sense, polyclonal antibodies, monoclonal antibodies, multi-specific antibodies, chimeric antibodies, anti-idiotype antibodies, and fragments thereof, e.g., Fv fragments Fab′ fragments, F(ab′)₂ and Fab fragments, as well as other conjugates or functional equivalents produced by recombination (e.g., chimeric antibodies, humanized antibodies, multifunctional antibodies, bispecific or oligospecific antibodies, single chain antibodies, scFV, diabody, sc(Fv)₂(single chain (Fv)₂), and scFv-Fc). Furthermore, such an antibody may be covalently bound, or recombinantly fused with an enzyme such as alkaline phosphatase, horseradish peroxidase, or a galactosidase. Antibodies to CD98 or the like used in the present invention is sufficient if they bind to a protein such as CD98, respectively, regardless of the origin, type, shape or the like thereof. Specifically, known antibodies such as a non-human animal antibody (e.g., a mouse antibody, a rat antibody, or a camel antibody), a human antibody, a chimeric antibody, or a humanized antibody can be used. In the present invention, a monoclonal or polyclonal antibody can be utilized as an antibody, but a monoclonal antibody is preferable. It is preferable that an antibody binds specifically to each protein such as CD98.

As used herein, an “antigen” refers to any substrate which can be specifically bound with an antibody molecule. As used herein, an “immunogen” refers to an antigen which can initiate lymphocyte activation that produces an antigen-specific immunological response. As used herein, an “epitope” or an “antigen determinant” refers to a site in an antigen molecule to which an antibody or a lymphocyte receptor binds. A method for determining an epitope is well known in the art. When a primary sequence of a nucleic acid or an amino acid is provided, such an epitope can be determined by those skilled in the art using such a well-known conventional technique.

As used herein, “means” refers to any matter which can serve as a tool for attaining a certain objective (e.g., detection, diagnosis, or treatment). As used herein, “means for selective recognition (detection)” especially refers to means which can recognize (detect) a certain subject differently from others.

It is understood that an antibody of any specificity may be used as an antibody used herein, as long as pseudopositivity is decreased. Thus, an antibody used in the present invention may be a polyclonal antibody or a monoclonal antibody. As used herein, a “ligand” refers to a substance that specifically binds to a certain protein. Examples of ligands include lectin, an antigen, an antibody, a hormone, a neurotransmitter and the like, which specifically bind to a variety of receptor protein molecules on a cell membrane.

The detection agent, the agent for diagnosis or other pharmaceuticals of the present invention can be in a form of a probe and a primer. The probe and the primer of the present invention can specifically hybridize with a molecule such as CD98. As described herein, expression of a molecule such as CD98 is an indicator a normal cell or transformed cell in corneal endothelial cells and is useful as an indicator of the level of transformation. Thus, the probe and the primer according to the present invention can be used for identifying a normal cell or transformed cell among corneal endothelial cells and/or identifying the level of transformation. In one embodiment, the probe and the primer of the present invention is sufficient if they detect expression of a molecule such as CD98, and refer to a polymer consisting of a plurality of bases or base pairs such as deoxyribonucleic acids (DNAs) or ribonucleic acids (RNAs). It is known that a double-stranded cDNA can also be utilized in tissue in situ hybridization. The probe and the primer of the present invention also encompass such a double-stranded cDNA. Examples of the probe and the primer particularly preferable in detection of RNA in a tissue include an RNA probe (riboprobe).

As used herein, a “(nucleic acid) primer” refers to a substance required for initiation of a reaction of a polymer compound to be synthesized in a polymer synthesizing enzyme reaction. In a reaction of synthesizing a nucleic acid molecule, a nucleic acid molecule (e.g., DNA, RNA, or the like) complementary to a part of a sequence of a polymer compound to be synthesized can be used. As used herein, a primer can be used as marker detection means.

Examples of a nucleic acid molecule which is generally used as a primer include molecules having a nucleic acid sequence having a length of at least 8 consecutive nucleotides, which is complementary to a nucleic acid sequence of a gene of interest. Such a nucleic acid sequence can be a nucleic acid sequence with a length of preferably at least 9 consecutive nucleotides, more preferably at least 10 consecutive nucleotides, still more preferably at least 11 consecutive nucleotides, at least 12 consecutive nucleotides, at least 13 consecutive nucleotides, at least 14 consecutive nucleotides, at least 15 consecutive nucleotides, at least 16 consecutive nucleotides, at least 17 consecutive nucleotides, at least 18 consecutive nucleotides, at least 19 consecutive nucleotides, at least 20 consecutive nucleotides, at least 25 consecutive nucleotides, at least 30 consecutive nucleotides, at least 40 consecutive nucleotides, or at least 50 consecutive nucleotides. A nucleic acid sequence used as a probe includes nucleic acid sequences which are at least 70% homologous, more preferably at least 80% homologous, still more preferably at least 90% homologous, or at least 95% homologous to the aforementioned sequences. A sequence appropriate as a primer can vary depending on the nature of a sequence which is intended to be synthesized (amplified). Those skilled in the art can appropriately design a primer depending on the intended sequence. Design of such a primer is well known in the art. Designing may be performed manually or by using a computer program (e.g., LASERGENE, PrimerSelect, or DNAStar).

The primer according to the present invention can also be used as a primer set consisting of two or more of the primers.

The primer and the primer set according to the present invention can be utilized as a primer and a primer set in accordance with a conventional method in a known method for detecting a gene of interest by utilizing a nucleic acid amplification method such as a PCR method, an RT-PCR method, a real time PCR method, an in situ PCR method, a LAMP method or the like.

The primer set according to the present invention can be selected so that a nucleotide sequence of a protein of interest such as a molecule such as CD98 can be amplified by a nucleic acid amplification method such as a PCR method. The nucleic acid amplification method is well known. Selection of a primer pair in the nucleic acid amplification method is obvious to those skilled in the art. For example, primers can be selected in a PCR method so that one of two primers (primer pair) is paired with a plus chain of double-stranded DNA of a protein of interest, e.g., a molecule such as CD98, and the other primer is paired with a minus chain of the double-stranded DNA, and the latter primer is paired with an extended chain which is extended by the former primer. Further, in a LAMP method (WO 00/28082), three regions of F3c, F2c and F1c from the 3′ terminal side, and three regions of B1, B2 and B3 from the 5′ terminal side are defined, respectively, for a target gene, and these six regions can be used to design four types of primers. The primer of the present invention can be chemically synthesized based on the nucleotide sequences disclosed herein. Preparation of the primer is well known and can be performed in accordance with, for example, “Molecular Cloning, A Laboratory Manual 2^{nd} ed.” (Cold Spring Harbor Press (1989)) or “Current Protocols in Molecular Biology” (John Wiley & Sons (1987-1997)).

As used herein, a “probe” refers to a substance that can be means for retrieval, used in a biological experiment such as in vitro and/or in vivo screening. Examples thereof include, but are not limited to, a nucleic acid molecule comprising a specific base sequence or a peptide comprising a specific amino acid sequence, a specific antibody or a fragment thereof and the like. As used herein, the probe is used as means for marker detection.

Examples of a nucleic acid molecule which is generally used as a probe include nucleic acid molecules having a nucleic acid sequence with a length of at least 8 consecutive nucleotides, which is homologous or complementary to a nucleic acid sequence of a gene of interest. Such a nucleic acid sequence can be at least a nucleic acid sequence with a length of preferably at least 9 consecutive nucleotides, more preferably at least 10 consecutive nucleotides, still more preferably a at least 11 consecutive nucleotides, at least 12 consecutive nucleotides, at least 13 consecutive nucleotides, at least 14 consecutive nucleotides, at least 15 consecutive nucleotides, at least 20 consecutive nucleotides, at least 25 consecutive nucleotides, at least 30 consecutive nucleotides, at least 40 consecutive nucleotides, or at least 50 consecutive nucleotides. A nucleic acid sequence used as a probe includes nucleic acid sequences which are at least 70% homologous, more preferably at least 80% homologous, still more preferably at least 90% homologous, or at least 95% homologous to the aforementioned sequences.

In one embodiment, the detection agent of the present invention can be a labeled agent. Alternatively, the detection agent of the present invention may be an agent bound to a tag.

As used herein, a “label” refers to an entity (e.g., substance, energy, electromagnetic wave or the like) for distinguishing a molecule or substance of interest from others. Examples of a method of applying such a label include an RI (radioisotope) method, a fluorescence method, a biotin method, a chemiluminescence method and the like. When a plurality of markers of the present invention, or agents or means for capturing them is labeled by a fluorescence method, labeling is performed with fluorescent substances having different fluorescence maximum wavelengths. A difference in the fluorescence maximum wavelength is preferably 10 nm or more. When a ligand is labeled, any label can be used as long as the function is not affected, although Alexa™ Fluor is desirable as a fluorescent substance. Alexa™ Fluor is a water-soluble fluorescent dye obtained by modifying coumarin, rhodamine, fluorescein, cyanine or the like. Alexa™ Fluor is a series compatible with a wide range of fluorescent wavelengths, which is very stable, bright, and low in pH sensitivity as compared with other fluorescent dyes of corresponding wavelengths. Examples of a combination of fluorescent dyes with a fluorescent maximum wavelength of 10 nm or longer include a combination of Alexa™ 555 and Alexa™ 633, a combination of Alexa™ 488 and Alexa™ 555, and the like. When a nucleic acid is labeled, any label can be used as long as it can bind to a base portion thereof. It is preferable that a cyanine dye (e.g., Cy3, Cy5 or the like of CyDye™ series), a rhodamine 6G reagent, N-acetoxy-N2-acetylaminofluorene (AAF), AAIF (iodine derivative of AAF) or the like is used. Examples of fluorescent substances having a difference in fluorescence maximum wavelengths of 10 nm or more include a combination of Cy5 and a rhodamine 6G reagent, a combination of Cy3 and fluorescein, a combination of a rhodamine 6G reagent and fluorescein and the like. In the present invention, such a label can be utilized to modify a subject of interest to be detectable by detection means to be used. Such a modification is known in the art. Those skilled in the art can appropriately carry out such a method in accordance with a label and a subject of interest.

As used herein, a “tag” refers to a substance for selecting and sorting out a molecule by a specific recognition mechanism such as in receptor-ligand binding, more specifically a substance which plays a role of a binding partner for binding to a specific substance (e.g., a substance having a relationship such as biotin-avidin or biotin-streptavidin). Tags can be included in the category of “label”. For example, a specific substance with a tag bound thereto can be selected and sorted out by contacting the substance with a substrate with a binding partner of a tag sequence bound thereto. Such a tag or label is well known in the art. Representative tag sequences include, but are not limited to, a myc tag, a His tag, HA, an Avi, and the like. The marker or the marker detection agent of the present invention may be bound to such a tag.

In one aspect, the present invention provides a method for using a molecule such as CD98 as an indicator for identifying a cell with a high proliferation ability among corneal endothelial cells and/or the differentiation ability of a corneal endothelial cell, or a method of detecting or diagnosing a cell with a high proliferation ability among corneal endothelial cells and/or the differentiation ability of a corneal endothelial cell.

The method of the present invention can be implemented by performing, for example, a step of detecting a molecule such as CD98 or genes of these molecules in a living body, for using the molecule such as CD98 as an indicator for identifying a cell with a high proliferation ability among corneal endothelial cells and/or the differentiation ability of a corneal endothelial cell. For example, in such a case, a detection agent comprising a substance that binds to a molecule such as CD98, or genes of these molecules can be used. Such a detection agent is described herein. It is understood that those skilled in the art can implement the method of the present invention based on the description herein.

In the method of the present invention, the detection agent or the diagnostic agent of the present invention is contacted with a sample of interest to determine whether there is a molecule such as CD98 or genes of these molecules that is a sample of interest in the sample or to measure the level or amount thereof.

As used herein, “contact (contacted)” refers to physically proximating a substance to a polypeptide or a polynucleotide which can function as the marker, the detection agent, the agent for diagnosis, the ligand or the like of the present invention, either directly or indirectly. The polypeptide or the polynucleotide can be made to be present in many buffers, salts, solutions or the like. Contact includes placing a compound on, for example, a beaker, a microtiter plate, a cell culturing flask or a microarray (e.g., gene chip), containing a polypeptide encoding a nucleic acid molecule or a fragment thereof.

In one embodiment, a cell with a high proliferation ability which is a subject in the method of the present invention, is an undifferentiated cell.

In another embodiment, a cell with a high proliferation ability, which is a subject in the method of the present invention, is a stem cell.

In another aspect, a corneal endothelial cell, which is a subject in the method of the present invention, is a human cell.

In yet another embodiment, the proliferation ability of a corneal endothelial cell, which is a subject in the method of the present invention, is identified by a characteristic selected from the group consisting of colony forming ability, Ki-67 positivity and BrdU positivity.

In one embodiment of the present invention, the differentiated state can be diagnosed based on the method using an indicator of the present invention.

A specific method of detecting expression of a molecule such as CD98, or genes of these molecules is not particularly limited as long as it is a method which can detect expression of a molecule such as CD98 or genes of these molecules in a test sample (e.g., cell or the like). Examples thereof include a hybridization method, a nucleic acid amplification method, an antigen-antibody reaction method and the like.

Herein, a “test sample” only needs to be a sample that is a corneal endothelial cell, a cell of interest, or a substance derived therefrom, which is considered to contain a matter allowing gene expression. For example, a cell which is directly isolated from the corneal endothelium can be used. A cell of the corneal endothelium can be obtained by a known method (Koizumi N, Okumura N, Kinoshita S., Experimental Eye Research. 2012; 95:60-7.). Preferably, a cell obtained from a donor of the corneal endothelium, a corneal endothelial cell or the like can be used as a test cell sample. Further, a cultured cell containing a corneal endothelial cell which was differentiated and induced in vitro can be used as a sample. In vitro differentiation induction into a corneal endothelial cell can be implemented by performing differentiation treatment by a known method, for example, an AMED method while using a known cell such as an ES cell, an iPS cell, a bone marrow stromal cell or the like as a starting material <Ueno M, Matsumura M, Watanabe K, Nakamura T, Osakada F, Takahashi M, Kawasaki H, Kinoshita S, Sasai Y; Proc Natl Acad Sci USA. 103 (25): 9554-9559, 2006.>.

According to one embodiment of detection according to the present invention, expression of a molecule such as CD98 or genes of the molecules in a cell sample can be detected by hybridizing the probe according to the present invention with a nucleic acid sample (mRNA or a transcription product thereof) and directly or indirectly detecting a hybridization complex, i.e., a double-stranded nucleotide. The following can be referred to for a detailed procedure of a hybridization method: “Molecular Cloning, A Laboratory Manual 2^{nd} ed.” (Cold Spring Harbor Press (1989), particularly Section 9.47-9.58), “Current Protocols in Molecular Biology” (John Wiley & Sons (1987-1997), particularly Section 6.3-6.4), and “DNA Cloning 1: Core Techniques, A Practical Approach 2^{nd} ed.” (Oxford University (1995), see particularly section 2.10 for conditions).

Detection of expression of a molecule such as CD98 or genes of these molecules utilizing a hybridization method can be implemented by, for example, (a) contacting a polynucleotide derived from a test sample with the probe according to the present invention; and (b) detecting a hybridization complex. In the step (a), mRNA prepared from a test sample of interest or complementary DNA (CDNA) transcribed from the mRNA can be contacted with the probe as the polynucleotide derived from a test cell sample. In a method of detection using a probe, the probe can be labeled for use. Examples of the label include labels utilizing radioactivity (e.g., ³²P, ¹⁴C, and ³⁵S), fluorescence (e.g., FITC and europium), an enzyme reaction such as chemiluminescence (e.g., peroxidase and alkaline phosphatase) or the like. A hybridization product can be detected using a well-known method such as Northern hybridization, Southern hybridization, colony hybridization, or the like. Since a cell from which a hybridization complex was detected is a cell expressing a molecule such as CD98, the cell can be determined to have a high proliferation ability (an undifferentiated cell, a precursor cell, a stem cell or the like) and/or a high differentiation ability.

According to another embodiment of detection according to the present invention, expression of a molecule such as CD98 or genes of these molecules in a sample can be detected by amplifying a nucleic acid sample (mRNA or a transcription product thereof) by a nucleic acid amplification method using the primer or the primer set according to the present invention and detecting the amplification product.

Detection of expression of a molecule such as CD98 or genes of these molecules utilizing a nucleic acid amplification method can be implemented, for example, by (i) implementing a nucleic acid amplification method using the primer or the primer set according to the present invention while using a polynucleotide derived from a test sample as a template; and (ii) detecting the formed amplification product.

In step (i), mRNA prepared from a test sample of interest or complementary DNA (CDNA) transcribed from the mRNA can be used as a template. An amplification product can be detected using a nucleic acid amplification method such as a PCR method, an RT-PCR method, a real time PCR method, or a LAMP method. A cell from which an amplification product is detected is highly likely a normal corneal endothelial cell for a normal corneal endothelial cell marker and highly likely a transformed corneal endothelial cell for a transformed corneal endothelial cell marker. Thus, the cell can be determined to be a normal or transformed cell.

According to another embodiment of detection according to the present invention, whether a contained cell is a normal or transformed cell and/or the ratio thereof in a sample can be determined by contacting the antibody according to the present invention with the sample to detect an antigen-antibody reaction.

Detection of expression of a molecule such as CD98 utilizing an antigen-antibody reaction can be implemented, for example, by the following steps: (I) contacting a protein derived from a test cell sample with the antibody according to the present invention; and (II) measuring an antigen-antibody complex. A method of detecting an antigen-antibody reaction is well known to those skilled in the art. For example, a molecule such as CD98 in a test cell sample which is thought to contain a dopaminergic neuron proliferative progenitor cell can be detected by an immunological method. As the immunological method, a known method such as an immunohistological staining method, an enzyme immunometric assay, a Western blotting method, an agglutination method, a competition method, or a sandwich method can be applied to a sample obtained by subjecting a cell sample to an appropriate treatment as needed such as separation of a cell or an extraction operation. The immunohistological staining method can be performed, for example, by a direct method using a labeled antibody, an indirect method using a labeled antibody to the above antibody, or the like. As a labeling agent, a known labeling substance such as a fluorescent substance, a radioactive substance, an enzyme, a metal, or a dye can be used.

Since a cell from which an antigen-antibody complex is detected is a cell expressing a molecule such as CD98, the cell can be determined to have high proliferation ability (an undifferentiated cell, a precursor cell, a stem cell or the like) and/or high differentiation ability. It is desirable that cells with a high proliferation ability (an undifferentiated cell, a precursor cell, a stem cell, or the like) are of high purity for use in therapy of a disease requiring transplantation of the corneal endothelium such as bullous keratopathy, corneal edema, leukoma corneae, particularly corneal dystrophy, or a corneal endothelial disorder caused by trauma or intraocular surgery, or other specified corneal endothelial diseases (Fuchs endothelial corneal dystrophy, posterior polymorphous corneal endothelial dystrophy etc.).

Instead of performing each of the aforementioned detection steps once, the steps can be repeated or combined to enhance the precision of detection or selection of the level of normal/transformed cells. Thus, when such an embodiment is used, the level of normal/transformed cells can be detected or selected more precisely by performing the aforementioned steps two or more times according to the detection method according to the present invention.

Further, the precision of detection or selection of the level of normal/transformed cells can be enhanced by concurrently using another marker gene, preferably a proliferation marker gene (e.g., Ki-67, BrdU or the like), detection agent thereof or the like.

As used herein, “diagnosis” refers to identification of a variety of parameters associated with a disease, disorder, condition or the like in a subject (e.g., living body, cell or the like) to determine the current or future status of such a disease, a disorder, a condition or the like. By using the method, the apparatus or the system of the present invention, the state in a cell a body, or the like can be examined. Such information can be used to select a variety of parameters such as a disease, a disorder, or a condition in a subject, a formulation or a method for treatment or prevention to be administered. As used herein, in a narrow sense, “diagnosis” refers to diagnosis of the current status, while encompassing “early diagnosis”, “presumptive diagnosis”, “advance diagnosis” and the like in a broad sense. Since the diagnosis method of the present invention, in principle, can utilize what has come from a body (e.g., cells) and can be implemented without a healthcare professional such as a doctor, it is industrially useful. For example, a cell can be diagnosed by using a substance that binds to a marker of the present invention to identify a normal corneal endothelial cell and a transformed corneal endothelial cell. As used herein, “presumptive diagnosis, advance diagnosis or diagnosis” particularly may be called “assistance” in order to clarify that the method can be implemented without a healthcare professional such as a doctor.

A procedure of formulating an agent for diagnosis or the like of the present invention as a pharmaceutical or the like is known in the art and described, for example, in Japanese Pharmacopoeia, U.S. Pharmacopoeia, and other countries' Pharmacopoeias. Thus, those skilled in the art can determine the amount of the agent for diagnosis to be used from the descriptions herein without undue experiments.

An antibody used in the present invention can be produced as follows.

An antibody used in the present invention (e.g., anti-CD98 antibody or the like) can be obtained as a polyclonal or monoclonal antibody using known means. As an antibody used in the present invention, a monoclonal antibody derived from a mammal is especially preferable. Monoclonal antibodies derived from a mammal include monoclonal antibodies produced by a hybridoma, monoclonal antibodies produced by a host transformed with an expression vector comprising an antibody gene by a genetic engineering procedure and the like.

As one example, a method for preparing a monoclonal antibody is described below. The monoclonal antibody can be prepared by preparing a hybridoma by cell fusion between an antibody producing cell obtained from an animal immunized with an antigen and a myeloma cell, and selecting a clone producing an antibody which specifically inhibits the activity of a molecule such as CD98 from the resulting hybridoma.

The full amino acid sequence of a protein such as a mature protein, such as a molecule such as CD98 used as an antigen in immunization of an animal, or a fragment thereof having immunogenicity, can be used as an immunogen. Further, it is preferable to use a peptide consisting of any 10 or more in an amino acid sequence of a protein of the marker of the present invention as an antigen, as a monoclonal antibody for specifically detecting a protein that is present on a cell surface. An antigen can be designed similarly for any other agent of the present invention (e.g., a molecule such as CD98 as well as proteins corresponding thereto or the like).

After binding to the resulting molecule such as CD98 for an antigen, an adjuvant is added. Examples of the adjuvant include a Freund complete adjuvant, a Freund incomplete adjuvant, and the like. Any of them may be mixed.

The antigen obtained as described above is administered to a mammal such as a mouse, a rat, a horse, a monkey, a rabbit, a goat, or sheep. Any method can be used for immunization as long as it is an existing method, but immunization is mainly performed by intravenous injection, subcutaneous injection, intraperitoneal injection or the like. Further, an immunization interval is not particularly limited. Immunization is performed at an interval of a few days to a few weeks, preferably at an interval of 4 to 21 days.

After 2 to 3 days from the day of last immunization, an antibody producing cell is collected. Examples of the antibody producing cell include a spleen cell, a lymph node cell, and a peripheral blood cell. However, a spleen cell is generally used. As the immunization amount of an antigen, for example, 100 μg of antigen is used once per mouse.

A monoclonal antibody producing hybridoma can be basically produced as follows using a known technique. First, a protein of interest (e.g., a protein such as a molecule such as CD98) is used as a sensitizing antigen, which is immunized according to a common immunization method. An immune cell obtained from an immunized animal is fused with a known parent cell by a common cell fusion method to obtain a hybridoma. Further, by screening a cell producing an antibody of interest from this hybridoma by a common screening method, a hybridoma producing an antibody of interest can be selected.

Specifically, production of the monoclonal antibody is performed, for example, as follows. First, a protein of interest used as a sensitizing antigen for obtaining an antibody can be obtained by expressing a gene of interest (a molecule such as CD98 or the like). A base sequence of the gene of interest and an amino acid sequence of protein is described in other parts herein (e.g., disclosed in SEQ ID NOs: 1-16). That is, after a genetic sequence encoding the gene of interest is inserted into a known expression vector to transform an appropriate host cell, a protein of interest can be purified from the host cell or the culture supernatant by a known method. Further, a purified natural protein can be similarly used. Further, as used in the present invention, a fusion protein obtained by fusing a desired partial polypeptide of a protein of interest with a different polypeptide can also be utilized as an immunogen. In order to produce the fusion protein to be used as an immunogen, for example, an Fc fragment of an antibody, a peptide tag or the like can be utilized. A vector expressing the fusion protein can be produced by fusing genes encoding two or more types of desired polypeptide fragments in frame and inserting the fused gene in an expression vector. A method of producing the fusion protein is described in Molecular Cloning 2nd ed. (Sambrook, J et al., Molecular Cloning 2nd ed., 9.47-9.58, Cold Spring Harbor Lab. press, 1989).

The protein of interest purified in this manner (e.g., a protein of a molecule such as CD98) can be used as a sensitizing antigen used in immunization of a mammal. A partial peptide of a protein of interest can also be used as a sensitizing antigen. For example, the following peptides can be used as a sensitizing antigen: a peptide obtained by chemical synthesis based on an amino acid sequence of a protein of interest (a molecule such as CD98 or the like); a peptide obtained by incorporating a part of a gene of interest (a molecule such as CD98 or the like) into an expression vector to express the gene; and a peptide obtained by degrading a protein of interest (a molecule such as CD98) with a protease.

The region and size of a protein used as a partial peptide are not limited. An exemplary region can be selected from an amino acid sequence constituting an extracellular domain of a molecule such as CD98. It is preferable that the number of amino acids constituting a peptide used as a sensitizing antigen is at least 3 or more, for example, 5 or more, or 6 or more. More specifically, a peptide of 8 to 50, preferably 10 to 30 residues can be used as a sensitizing antigen.

In one embodiment, examples of the detection kit according to the present invention include a detection kit for detection of an embodiment according to the present invention, specifically, a kit for detecting expression of a molecule such as CD98, comprising at least the probe according to the present invention. This probe may be labeled. This kit for detection detects expression of a molecule such as CD98 by a hybrid forming method. Thus, a detection method of a first embodiment can further comprise a variety of reagents for carrying out the hybrid forming method, for example a substrate compound used in detection of a label, a hybridization buffer, instruction manual, and/or an instrument as desired.

The detection kit of this embodiment according to the present invention may further comprise a probe, a primer, a primer set, or an antibody which can detect expression of a differentiation marker gene (e.g., Ki-67, BrdU or the like) other than a molecule such as CD98, in order to perform highly precise detection. These probe, primer, primer set and antibody may be labeled. This kit for detection further detects expression of a differentiation marker gene other than a molecule such as CD98 by any of a hybrid forming method, a nucleic acid amplification method, and an antigen-antibody reaction method.

In another embodiment, examples of the kit for detection according to the present invention include a detection kit for carrying out detection of another embodiment according to the present invention, specifically, a kit for detecting expression of a molecule such as CD98, comprising at least the primer according to the present invention or the primer set according to the present invention. This kit for detection detects expression of a molecule such as CD98 by a nucleic acid amplification method. Thus, a detection method of a second embodiment may further comprise a variety of reagents for carrying out a nucleic acid amplification method, for example a buffer, an internal standard which indicates that PCR can progress normally, an instruction manual, and/or an instrument as desired.

The detection kit of this embodiment according to the present invention may further comprise a probe, a primer, a primer set, or an antibody which can detect expression of a differentiation marker gene other than a molecule such as CD98 for high precision detection. These probe, primer, primer set, and antibody may be labeled. This kit for detection further detects expression of a differentiation marker other than a molecule such as CD98 by any of a hybrid forming method, a nucleic acid amplification method, and an antigen-antibody reaction method.

In a further embodiment, examples of the detection kit according to the present invention include a detection kit for carrying out detection of the further embodiment according to the present invention, specifically, a kit for detecting a protein of a molecule such as CD98, comprising at least the antibody according to the present invention. This antibody may be labeled. This kit for detection detects expression of a molecule such as CD98 by detecting an antigen-antibody reaction. The detection method of this embodiment may further comprise a variety of reagents for carrying out an antigen-antibody reaction, for example a secondary antibody, a coloring reagent, a buffer, an instruction manual, and/or an instrument used in an ELISA method or the like as desired.

In this embodiment, the detection kit according to the present invention may further comprise a probe, a primer, a primer set, or an antibody which can detect expression of a differentiation marker other than a molecule such as CD98 for highly precise detection. These probe, primer, primer set, and antibody may be labeled. This kit for detection further detects expression of a differentiation marker other than a molecule such as CD98 by any of a hybrid forming method, a nucleic acid amplification method, and an antigen-antibody reaction method.

It can be understood that these kit, composition or system can use a marker in a sample derived from any subject, agents specifically interacting with the marker, or means selectively recognizing the marker as long as the marker of the present invention (e.g., a molecule such as CD98) can be identified. Thus, it is understood that not only an agent or means specifically described herein, but also any equivalent agent or means known in the art can be used.

In one embodiment, the agent used in the present invention is selected from the group consisting of a nucleic acid molecule, a polypeptide, a fat, a glycan, an organic small molecule and a composite molecule thereof, and the agent is preferably a protein or a composite molecule (e.g., a glycoprotein, a lipoprotein, or the like). Preferably, the agent is an antibody (e.g., a polyclonal antibody or a monoclonal antibody). It is preferable that such an agent is labeled, or can be labeled. This is because diagnosis would be facilitated.

In a preferred embodiment of the present invention, means to be used is selected from the group consisting of a mass spectrometry apparatus, a nuclear magnetic resonance measuring apparatus, an X-ray analysis apparatus, SPR, chromatography (e.g., HPLC, thin layer chromatography, or gas chromatography), an immunological means (e.g., Western blotting, EIA (enzyme immunoassay), RIA (radioimmunoassy), or ELISA (enzyme linked immunosorbent assay)), a biochemical means (e.g., pI electrophoresis, Southern blotting, or two-dimensional electrophoresis), an electrophoresis instrument, a chemical analysis instrument, a fluorescent two-dimensional differential electrophoresis method (2DE-DIGE), an isotope-coded affinity tag (ICAT), a tandem affinity purification method (TAP method), a physical means, laser microdissection and a combination thereof.

In a preferable embodiment of the present invention, the system or the kit of the present invention further comprises a standard of a marker. It is preferable that such a standard is used in order to confirm whether means for detecting a marker (an agent specifically interacting with the marker, means selectively recognizing the marker or the like) is functioning normally.

In a preferable embodiment, the present invention can further comprise means for purifying a sample as a subject. Examples of such purification means include chromatography and the like. Since the precision of diagnosis can be enhanced by purification, the purification means can be used, but it is not essential, in a preferred embodiment.

### PREFERRED EMBODIMENTS

Preferred embodiments of the present invention are disclosed below. Embodiments provided below are provided for better understanding of the present invention. It is understood that the scope of the present invention should not be limited to the following descriptions. Thus, it is apparent that those skilled in the art can make appropriately modifications within the scope of the present invention while referring to the descriptions herein.

### (Normal Cell/Transformed Cell Markers)

In one aspect, the present invention provides a novel marker for identifying a normal cell and a transformed cell in corneal endothelial cells, comprising at least one marker selected from a normal corneal endothelial cell marker and/or transformed corneal endothelial cell marker.

The normal corneal endothelial cell marker provided in the present invention includes CD166, HLA-A2, CD66a, CD66c, CD66d, CD66e, CD98, CD59, CD54, CD340, CD47, EGF-R, CD29, CD74, CD165, CD221, CD49a, SSEA-4, CD130, CD49f, and the like.

Further, the transformed corneal endothelial cell marker provided in the present invention includes CD26, CD9, CD49b, CD49e, CD13, CD99, CD105, CD63, CD58, CD201, CD56, CD44, CD55, CD71, CD73, CD91, HLA-DQ, CD164, CD49d, CD49c, CD90, MICA/B, CD46, CD140b, CD146, CD147, CD81, CD151, CD200, CD10 and the like.

Most of these markers are cell surface markers. Since the markers are cell surface markers, it is understood that they can be readily used in FACS or the like and facilitate cell analysis.

The markers of the present invention may include at least one transformed corneal endothelial cell marker and at least one normal corneal endothelial cell marker. Since both normal cells and transformed cells can be judged with a positive reaction of marker expression by providing (multiple) markers indicating two types of tendencies, analysis is further facilitated.

Higher level of marker expression is more advantageous for use as a marker. This is because normal and transformed cells would be readily identifiable. For humans, the levels of appropriate strong expression as a marker include, but are not limited to, levels exceeding about 1 time, such as at least about 1.1 times or more, at least about 1.2 times or more, at least about 1.3 times or more, at least about 1.4 times or more, at least about 1.5 times or more, at least about 1.6 times or more, at least about 1.7 times or more, at least about 1.8 times or more, at least about 1.9 times or more, at least about 2 times or more, at least about 2.5 times or more, at least about 3 times or more, at least about 4 times or more, and at least about 5 times or more. Further, one or more markers may be used. Please refer to Tables 2-3 for levels of expression. Table 4 shows preferred markers (CD98, CD166, and CD340 for normal cells, and CD9, CD49e, CD44, and CD73 for transformed cells), but the markers are not limited thereto. Further, it is understood that any of one, two, three, four, five, six, or all seven markers described in Table 4 may be combined.

In one preferred embodiment, CD98, CD166, and CD340 are advantageous as normal corneal endothelial cell markers. One, two or three of these markers can be used. Although not wishing to be bound by any theory, since a very significant difference in expression levels were observed between normal and transformed corneal endothelial cells for these markers, utility as markers thereof can be expected.

In another preferred embodiment, CD9, CD49e, CD44 and CD73 are advantageous as transformed corneal endothelial cell markers. One, two, three, or four of these markers can be used. Although not wishing to be bound by any theory, since a very significant difference in expression levels were observed between normal and transformed corneal endothelial cells for these markers, utility as markers thereof can be expected.

In a still more preferable embodiment, the markers of the present invention include at least one molecule selected from the group consisting of CD98, CD166, and CD340 as a normal corneal endothelial cell marker and at least one molecule selected from the group consisting of CD9, CD49e, CD44, and CD73 as a transformed corneal endothelial cell marker.

In a still more preferable embodiment, the markers of the present invention include at least one marker selected from the group consisting of CD166 and CD73. An even more significant difference in expression levels were observed between normal and transformed corneal endothelial cells, utilize as a marker thereof is expected even more.

In another aspect, the present invention provides a nucleic acid of a gene that can be used as a marker of the present invention and/or a protein encoded by the nucleic acid as a marker. In this case, the present invention provides a detection agent for identifying a normal corneal endothelial cell and a transformed corneal endothelial cell, comprising a substance that binds to the marker of the present invention. It is understood that any form described herein can be utilized as the marker of the present invention. Alternatively, the present invention provides an agent for diagnosis for identifying a normal corneal endothelial cell and a transformed corneal endothelial cell, comprising a substance that binds to a marker of the present invention. Further, the present invention provides a method of diagnosis for identifying a normal corneal endothelial cell and a transformed corneal endothelial cell by using a substance that binds to a marker of the present invention. The detection agent or agent for diagnosis of the present invention can use a substance that binds to a marker of the present invention when a target cell is provided to detect or diagnose whether the target cell is normal or transformed based on information related to the state of a binding between the substance and the cell (e.g., the substance can be used as a label to detect a signal or the like associated with the label or a substance binding thereto).

In one specific aspect, the present invention provides a method of detection for identifying a normal corneal endothelial cell and a transformed corneal endothelial cell, comprising: applying a sample that binds to a marker of the present invention to a sample comprising a corneal endothelial cell, and detecting a binding between the substance and the corneal endothelial cell. Alternatively, the present invention provides a method of diagnosing a corneal endothelial cell, comprising: applying a substance that binds to a marker of the present invention to a sample comprising the corneal endothelial cell; and detecting a binding between the substance and the cell, wherein a normal corneal endothelial cell and a transformed corneal endothelial cell are identified by judging the presence of the binding.

In one embodiment, the substance may be an antibody or an antigen binding fragment thereof, nucleic acid primer, probe or the like.

In a certain embodiment, a cell targeted by an agent for diagnosis or a detection agent of the present invention is a cell that is or was present in a living body. Alternatively, a cell targeted by an agent for diagnosis or detection agent of the present invention may be a subcultured cell or the like, which is not or has not been in a living body.

In a certain embodiment, an agent for diagnosis or method of diagnosis of the present invention can be used to diagnose a corneal endothelial disease. For instance, it is possible to diagnose that a corneal endothelial disease associated with a transformed cell or fibroblast has occurred or there is an indication thereof when an agent for diagnosis or a method of diagnosis of the present invention is used to study the state of a cell that is or was present in a living body and a transformed corneal endothelial cell is found, or when, in a certain embodiment, expression of a transformed corneal endothelial cell marker of the present invention is found at a certain level or higher. When studying in a living body, the state of cells can be diagnosed, for example, before surgery, during surgery or the like. Such a certain level can be appropriately determined by those skilled in the art using common general knowledge in the art such as statistics based on the information herein. Corneal endothelial disorders that may be the subject of detection or diagnosis of the present invention include, but are not limited to, primary disorders (diseases targeting endothelial cells such as corneal endothelial dystrophy (cornea guttata, Fuchs corneal endothelial dystrophy, posterior polymorphous corneal dystrophy, iridocorneal endothelial syndrome, and congenital hereditary corneal endothelial dystrophy), viral diseases (cytomegalovirus corneal endotheliitis, herpes virus simplex corneal endotheliitis and the like), exfoliation syndrome, post-corneal transplantation rejection and the like), secondary disorders (inflammation or physical damage involving an external factor such as keratouveitis, interstitial keratitis, post-cataract surgery, post-retina/vitreous body surgery, post-glaucoma surgery (filtration surgery, peripheral iridotomy), glaucoma attack, long-term contact lens usage, corneal trauma, and birth trauma), final symptoms such as bullous keratopathy. Particularly important corneal endothelial diseases include, but are not limited to, diseases involving fibrotic change of corneal endothelial cells including corneal dystrophy such as Fuchs corneal endothelial dystrophy and posterior polymorphous corneal endothelial dystrophy, corneal endothelial disorder due to trauma or intraocular surgery, and bullous keratopathy.

In one embodiment, an agent for diagnosis or method of diagnosis of the present invention determines that a transformed corneal endothelial cell is comprised when at least one transformed corneal endothelial cell marker is expressed. Alternatively, in another embodiment of the present invention, a corneal endothelial cell is diagnosed as having an irreversible degeneration when at least one transformed corneal endothelial cell marker is expressed.

In one embodiment, the present invention may further comprise an agent for staining the above-described binding substance used in the present invention. The binding state can be made more visible by such staining of a binding substance. Any agent known in the art can be used as an agent used in such staining, as long as the agent is capable of staining.

Any method known in the art can be used as the staining technique. For instance, use of an antibody is known in the art for detecting the presence or overexpression of a specific protein. Since a transformed corneal endothelial cell marker is expressed or have significantly increased expression in a transformed corneal endothelial cell in the present invention, an antibody specific to the transformed corneal endothelial cell marker or normal corneal endothelial cell marker of the present invention can be used for the detection of the expression and the detection of transformed state thereby. The aforementioned technique is not limited thereto and includes western blot, dot blot, precipitation, coagulation, ELISA assay, immunohistochemistry, in situ hybridization, flow cytometry in various tissues and body fluids, and various other sandwich assays. Such techniques are well known in the art. For example, see U.S. Pat. No. 5,876,949 (the entirety thereof is incorporated herein by reference). It is necessary to lyse a cell and incubate the cell with an antibody thereof when using the antibody specific to an intracellular epitope of a transformed corneal endothelial cell marker or normal corneal endothelial cell marker of the present invention. The aforementioned technique is performed in a whole cell lysate or a separated transformed corneal endothelial cell or normal corneal endothelial cell for testing by immunoprecipitation. Alternatively as another technique, for example, immunohistological straining requires the entire cell and further requires a cell layer with a specific cell density. The aforementioned test requires an antibody specific to an extracellular epitope of a transformed corneal endothelial cell marker or normal corneal endothelial cell marker of the present invention. Since a transformed corneal endothelial cell marker or a normal corneal endothelial cell marker is a surface antigen, cytolysis is generally considered unnecessary.

Further, an antibody specific to a transformed corneal endothelial cell marker or a normal corneal endothelial cell marker of the present invention can be utilized for detecting or diagnosing the state of a corneal endothelial cell. A processing method therefor such as immunohistological staining or immunofluorescence microscopy is well known in the art. Such a method can be used for visualizing a transformed corneal cell or normal corneal endothelial cell. For example, see U.S. Pat. No. 5,514,554 (the entirety thereof is incorporated herein by reference). For example, expression of a transformed corneal endothelial cell marker or a normal corneal endothelial cell marker of the present invention can be detected by using an antibody that can detect the entire protein of the transformed corneal endothelial cell marker or normal corneal endothelial cell marker of the present invention or a fragment of a protein thereof. Alternatively, other methods of detecting expression of a transformed corneal endothelial cell marker or normal corneal endothelial cell marker of the present invention include detection of DNA or RNA sequence encoding the transformed corneal endothelial cell marker or normal corneal endothelial cell marker of the present invention. Such a technique for detecting a DNA or RNA sequence is well known in the art and any method can be used.

In this case, an antibody specific to a transformed corneal endothelial cell marker or normal corneal endothelial cell marker of the present invention can be labeled by a covalent or non-covalent bond or linkage or binding by numerous known detectable labels such as fluorescent, radioactive, enzymatic substance or the like in order to detect the expression or distribution change of the transformed corneal endothelial cell marker or normal corneal endothelial cell marker of the present invention. Alternatively, a secondary antibody specific to an antibody specific to a transformed corneal endothelial cell marker or normal corneal endothelial cell marker of the present invention can be labeled with any suitable and detectable label known in the art and used to detect an antibody specific to the transformed corneal endothelial cell marker or normal corneal endothelial cell marker of the present invention.

In one embodiment, diagnosis in a living body can be performed as follows. As one example, an antibody specific to a fluorescently labeled transformed corneal endothelial cell marker or normal corneal endothelial cell marker can be administered into the anterior chamber (or into a vessel) and observed in a living body by a fluorescent microscope or the like. However, diagnostic methods in a living body are not limited thereto. Any other form can be used.

In another aspect, the present invention provides a method of using a marker of the present invention as an indicator for identifying a normal corneal endothelial cell and a transformed corneal endothelial cell. It is understood that any form described herein can be utilized as the marker of the present invention.

(Method of Preparing a Corneal Endothelial Cell Sample with Elevated Ratio of Normal Corneal Endothelial Cells/Method of Preparing Purified Corneal Endothelial Cells)

In another aspect, the present invention provides a method of preparing a corneal endothelial cell sample with an elevated ratio of normal corneal endothelial cells, comprising a) providing a sample comprising corneal endothelial cells, and b) elevating the ratio of normal corneal endothelial cells while using the marker of the present invention as an indicator. It is understood that any form described herein, including (Normal cell/transformed cell markers), can be utilized as the marker of the present invention.

In another aspect, the present invention provides a method of preparing purified corneal endothelial cells, wherein the method comprises: inducing corneal endothelial cells from undifferentiated cells; and elevating a ratio of the corneal endothelial cells while using the marker of the present invention as an indicator. It is understood that any form described herein, including (Normal cell/transformed cell markers) can be utilized as the marker of the present invention.

Examples of the purification method include, but are not limited to, a method of fractionating cells expressing a marker of interest in fluorescence activated cell sorter (FACS), magnetic-activated cell sorting (MACS) and the like.

One specific method includes, but is not limited to, the following: reacting a cultured corneal endothelial cell with an antibody to a normal cell or transformed cell marker and then separating and collecting either a normal cell or transformed cell depending on the level of intensity of expression of the normal cell or transformed cell marker of interest by a fluorescence activated cell sorter.

In one embodiment, undifferentiated cells are induced pluripotent stem (iPS) cells or embryonic stem (ES) cells.

In another aspect, the present invention provides a composition comprising a cell prepared by the method of the present invention.

In still another aspect, the present invention provides use of the composition of the present invention for cornea therapy. The subject of cornea therapy may include, but not limited to, corneal endothelial diseases.

### (Purity Test)

In another aspect, the present invention provides an agent for determining purity when making a sample comprising corneal endothelial cells, comprising a substance that binds to the marker of the present invention. Alternatively, the present invention provides a method of determining purity when making a sample comprising corneal endothelial cells, comprising applying a substance that binds to the marker of the present invention to a sample comprising a corneal endothelial cell and detecting a binding between the substance and the corneal endothelial cell, wherein a normal corneal endothelial cell and a transformed corneal endothelial cell are identified by determining the presence of the binding. The present invention further provides a method of therapy or prevention using a cell prepared by the method of determining purity of the present invention. In one embodiment, the therapy or prevention is for a cornea. In one embodiment, a subject of the therapy and prevention includes corneal endothelial diseases. It is understood that any technique and embodiment described herein may be applied to the diseases, therapy and prevention.

Further, in another aspect, the present invention provides a method of using the marker of the present invention as an indicator for determining purity when making a sample comprising corneal endothelial cells.

Methods of determining purity include, but are not limited to, measuring expressed antigens by flow cytometry or the like and dividing the result as the ratio of the whole to determine the purity, observation with a fluorescence microscope after immunostaining, magnetic-activated cell sorting (MACS), western blot, PCR and the like.

One specific method includes, but is not limited to the following: reacting a cultured corneal endothelial cell with an antibody to a normal or transformed cell marker and then determining the ratio of normal cells or transformed cells in a cell population by the level of intensity of expression of a normal cell or transformed cell marker of interest by flow cytometry.

It is understood that any form described herein, including (Normal cell/transformed cell markers), can be utilized as the marker of the present invention used in purity determining techniques.

### (General Techniques)

Molecular biological methods, biochemical methods, and microbiological methods used herein are well known and commonly used in the art, which are described in, for example, Sambrook J. et al. (1989). Molecular Cloning: A Laboratory Manual, Cold Spring Harbor and the 3rd Ed. (2001) thereof; Ausubel, F. M. (1987). Current Protocols in Molecular Biology, Greene Pub. Associates and Wiley-Interscience; Ausubel, F. M. (1989). Short Protocols in Molecular Biology: A Compendium of Methods from Current Protocols in Molecular Biology, Greene Pub. Associates and Wiley-Interscience; Innis, M. A. (1990). PCR Protocols: A Guide to Methods and Applications, Academic Press; Ausubel, F. M. (1992). Short Protocols in Molecular Biology: A Compendium of Methods from Current Protocols in Molecular Biology, Greene Pub. Associates; Ausubel, F. M. (1995). Short Protocols in Molecular Biology: A Compendium of Methods from Current Protocols in Molecular Biology, Greene Pub. Associates; Innis, M. A. et al. (1995). PCR Strategies, Academic Press; Ausubel, F. M. (1999). Short Protocols in Molecular Biology: A Compendium of Methods from Current Protocols in Molecular Biology, Wiley, and annual updates; Sninsky, J. J. et al. (1999). PCR Applications: Protocols for Functional Genomics, Academic Press, Experimental Medicine, separate volume, “Gene Introduction & Expression Analysis Experimental Method” Yodosha Co., Ltd., 1997 and the like. The relevant portion thereof (can be the entire document) is incorporated herein by reference.

A DNA synthesis technique for producing an artificially synthesized gene and nucleic acid chemistry are described, for example, in Gait, M. J. (1985). Oligonucleotide Synthesis: A Practical Approach, IRL Press; Gait, M. J. (1990). Oligonucleotide Synthesis: A Practical Approach, IRL Press; Eckstein, F. (1991). Oligonucleotides and Analogues: A Practical Approach, IRL Press; Adams, R. L. et al. (1992). The Biochemistry of the Nucleic Acids, Chapman & Hall; Shabarova, Z. et al. (1994). Advanced Organic Chemistry of Nucleic Acids, Weinheim; Blackburn, G. M. et al. (1996). Nucleic Acids in Chemistry and Biology, Oxford University Press; and Hermanson, G. T. (1996). Bioconjugate Techniques, Academic Press, and the like. The relevant portion thereof is incorporated herein by reference.

For example, as used herein, the oligonucleotide of the present invention can also be synthesized by a standard method known in the art, such as by using an automated DNA synthesizer (e.g., a synthesizer commercially available from Biosearch, Applied Biosystems or the like). For example, a phosphorothioate-oligonucleotide can also be synthesized by the method of Stein et al. (1988, Nucl. Acids Res. 16:3209), and a methyl phosphonate-oligonucleotide can also be prepared by using a control pore glass polymer support (Sarin et al., 1988, Proc. Natl. Acad. Sci. USA 85:7448-7451).

Reference literatures such as scientific literatures, patents, and patent applications cited herein are incorporated herein by reference to the same extent that the entirety of each document is specifically described.

As described above, the present invention has been illustrated by showing preferable embodiments to facilitate understanding. The present invention is illustrated below based on Examples. The aforementioned illustration and the following Examples are not provided for the purpose of limiting the present invention, but for the sole purpose of exemplification. Thus, the scope of the present invention is not limited to embodiments and Examples specifically described herein and is limited only by the scope of claims.

### EXAMPLES

If necessary, animals used in the following Examples were handled in compliance with the standard set forth at Kyoto Prefectural University of Medicine or Doshisha University and the Declaration of Helsinki. Further, animals were fed and handled in accordance with the ARVO Statement for the Use of Animals in Ophthalmic and Vision Research. For reagents, products specifically described in Examples were used. However, equivalent products of other manufacturers (Sigma, Wako Pure Chemical Industries Co., Ltd., Nacalai Tesque, Inc., abcam, Santa Cruz Biotechnology, R & D Systems, Abnova, Assay Pro, Origene, Biobyt, Biorad, Cell Signaling Technology, GE Healthcare, IBL, and the like) can be used as substitutes.

### EXPERIMENTAL MATERIALS AND METHODS

### (Material)

### (Corneal Tissue)

All human corneal tissues used in the present experiment were imported from SightLife™ (Northwest Lions Foundation) of American Seattle Eye Bank. All monkey corneal tissues used were corneas of a cynomolgus monkey euthanized for other research purposes (Nissei Bilis Co., Ltd., Ohtsu, Japan, or Keari Co., Ltd., Wakayama, Japan). All corneas were preserved at 4° C. in a preservation medium (Optisol; Chiron Vision Corporation, Irvine, CA).

### (Cell Culture)

In primary culture of a human corneal endothelial cell, a Descemet's membrane including an endothelial cell layer was peeled off from a corneal tissue, and placed in 2 mg/ml Collagenase A (catalog No.: 70164923; Roche Applied Science, Penzberg, Germany) dissolved in OPTIMEM-I and incubated at 37° C. After 12 hours, the sample was centrifuged at 1000 rpm for 5 minutes to remove the supernatant. A culture medium was then added to a precipitated corneal endothelial cell mass for admixing. The entire amount was seeded on a 12-well plate coated with FNC Coating Mix (catalog No.: 0407; Athena Enzyme Systems, Baltimore, MD, USA). As a culture medium, OPTIMEM-I (catalog No.: 51985; Gibco-Invitrogen, Carlsbad, CA), to which 8% fetal bovine serum (catalog No.: 10437-028; fetal bovine serum; FBS; BioWest, France), 50 μg/ml Gentamicin (Invitrogen), and 10 μg/ml Y-27632 (Calbiochem, La Jolla, CA) were added, was used.

In primary culture of a monkey corneal endothelial cell, a Descemet's membrane including an endothelial cell layer was peeled off from a corneal tissue, and placed in 2 mg/ml Collagenase A (catalog No.: 70164923; Roche Applied Science, Penzberg, Germany) dissolved in DMEM (Gibco-Invitrogen) and incubated at 37° C. After 12 hours, the sample was centrifuged at 1000 rpm for 5 minutes to remove the supernatant. A culture medium was then added to a precipitated corneal endothelial cell mass for admixing. The entire amount was seeded on a 12-well plate coated with FNC Coating Mix (catalog No.: 0407; Athena Enzyme Systems, Baltimore, MD, USA). As a culture medium, DMEM (catalog No.: 12320; Gibco-Invitrogen), to which 10% FBS, 50 g/ml Gentamicin (catalog number: 15710-064; Invitrogen), and 2 ng/ml basic fibroblast growth factor (catalog No.: 13256-029; bFGF; Invitrogen) were added, was used.

As a human cornea, a cornea with the period before primary culture of less than 14 days was used. A previously reported system [Koizumi N et al., Exp Eye Res., 2012; 95:60-67; Koizumi N et al., Invest Ophthalmol Vis Sci . . . 2007; 48:4519-4526; Okumura N et al., Am J Pathol. 2012; 181:268-277] was used for culture of human and monkey corneal endothelial cells (CEC).

A medium was exchanged every 2 days. Subculture was performed when reaching 50 to 80% confluence. As a subculture method, cells were washed with Ca²⁺Mg²⁺-free PBS (PBS-; Nissui Pharmaceutical Co., Ltd., Tokyo, Japan), and TrypLE™ Select (catalog No.: 12563; Invitrogen) was added and incubated at 37° C. for 5 minutes. Cells were peeled off and recovered from the plate. After the cells were centrifuged at 1000 rpm for 5 minutes, a culture medium was added to make a cell suspension. Cells were seeded on a plate coated with FNC Coating Mix at a density of 1:2-3.

### (Immunostaining)

After immobilizing the culture cells, immunostaining was performed on cells by using CD73, CD166, or, as a functionally related marker, ZO-1, Na⁺/K⁺-ATPase for observation with a fluorescent microscope. Cultured monkey or human corneal endothelial cells were cultured in a 24 well plate (Sigma-Aldrich Co., St. Louis, MO) and immobilized for 10 minutes with 4% formaldehyde at room temperature (RT). The cells were incubated for 30 minutes with 1% bovine serum albumin (BSA). Immunohistological staining was then performed with Anti-Human CD73 (catalog number: 550256; BD Pharmingen) or Anti-Human CD166 (catalog number: 559260; BD Pharmingen), or a barrier function associated protein Z0-1 (Zymed Laboratories, Inc., South San Francisco, CA) or a pumping function associated protein Na+/K+-ATPase (Upstate Biotec, Inc., Lake Placid, NY). 1:300 dilution of each antibody was used in immunohistological staining. 1:1000 dilution of Alexa Fluor® 488 labeling Alexa Fluor® was used for secondary antibodies. Cellular nuclei were then stained with DAPI (Vector Laboratories, Inc., Burlingame, CA). Slides were then observed with a fluorescent microscope (BZ-9000, KEYENCE, Osaka, Japan).

### (RT-PCR)

PCR was performed with respect to CD166, CD73, and GAPDH by RT-PCR (Semi-quantitative reverse transcription polymerase chain reaction). A primer was purchased from oligonucleotide synthesizing firm, INVITROGEN and desalinated for use. RNEasy Mini Kit (QIAGEN, catalog number: 74106) was used for extraction of total RNA from cells. Reverse transcription reaction (42° C., 60 minutes) was performed on the extracted RNA with ReverTra Ace (TOYOBO (catalog number: TRT-101)), and CD166 and CD73 were amplified with TAKARA Taq Hot Strat Version (Takara Bio, catalog number: RR001A) with GAPDH as the internal standard. The same amount of cDNA was amplified by a PCR system (GeneAmp 9700; Applied Biosystems) and the following primer pairs.

*CD166-F:
(SEQ ID NO: 17)
CCCCAGAGGAATTTTTGTTTTAC
*CD166-R:
(SEQ ID NO: 18)
AGCCTGATGTTATCTTTCATCCA
*CD73-F:
(SEQ ID NO: 19)
GTTCCTGTAGTCCAGGCCTATG
*CD73-R:
(SEQ ID NO: 20)
ACATTTCATCCGTGTGTCTCAG
*GAPDH-F:
(SEQ ID NO: 21)
GAGTCAACGGATTTGGTCGT
*GAPDH-R:
(SEQ ID NO: 22)
TTGATTTTGGAGGGATCTCG

An amplified DNA fragment was detected by electrophoresis with 1.5% agarose gel (Nacalai Tesque, catalog number: 01149-76) and staining with ethidium bromide (Nacalai Tesque, catalog number: 14603-51).

### (Flow Cytometry)

Cultured human corneal endothelial or cultured monkey corneal endothelial cells were seeded on a culture dish coated with FNC Coating Mix and cultured for about 14 days until reaching confluence under conditions of 5% CO: at 37° C. Cells were peeled off with TrypLE™ Select and collected. Human Cell Surface Marker Screening Panel (BD Lyoplate™, BD Bio-sciences, Franklin Lakes, NJ) was used. A flow cytometer (BD FACSCanto™ MII (BD Biosciences, Franklin Lakes, NJ) was used in accordance with the manual to perform high-throughput surface antigen analysis. Further, cultured monkey corneal endothelium (BD AccuriTy C6 (BD Biosciences, Franklin Lakes, NJ)) was used to analyze surface antigens. 70% ethanol was added to cells peeled off and collected with TrypLE™ Select, and the cells were incubated for 2 hours at −20° C. to immobilize the cells. After washing the cells twice with PBS, 1% BSA was added. The cells were incubated for 15 minutes at room temperature for blocking. APCMouse anti-Human CD73 (catalog number: 560847; BD Pharmingen) or PEMouse anti-Human CD166 (catalog number: 560903; BD Pharmingen) were used and the cells were incubated for 30 minutes at room temperature. Cultured monkey corneal endothelium and immortalized human corneal endothelial cells were sorted by using FACS Aria II (BD Bio-sciences, Franklin Lakes, NJ). Collected cells were incubated at room temperature for 15 minutes with Purified Mouse anti-Human CD73 (catalog number: 550256; BD Pharmingen) as a primary antibody. Alexa Fluor 488-conjugated goat anti-rabbit IgG (1:1500) was used and incubated for 20 minutes at room temperature as a secondary antibody. The cells were sorted by the level of expression of CD73 and cultured in a 24-well plate (Sigma-Aldrich Co., St. Louis, MO).

### Reference Example 1

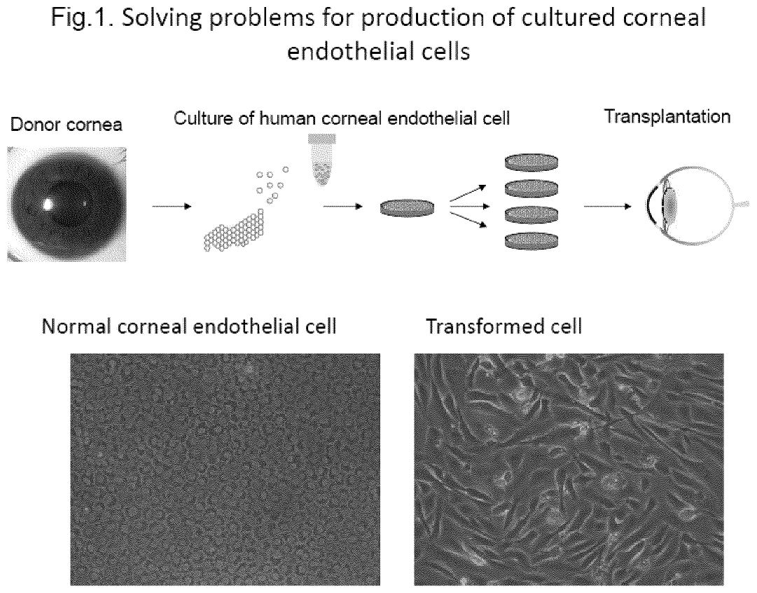

As shown in FIG. 1, the state of the cells when human corneal endothelial cells are separated from a donor cornea and cultured was observed in order to study the issues in conventional cultured corneal endothelial cell production. The experimental observation conditions are as described in the above-described section of (Experimental materials and methods).

As shown in FIG. 1, the left side shows corneal endothelium that could be cultured while maintaining a normal polygonal cellular form. However, when separated and cultured by a common method, the cells readily transform and change into a fibroblast-like form to lose cellular function as shown in the bottom right.

Production of corneal endothelial cells free of transformed cells is essential for clinical applications of cultured corneal endothelial cell transplantation. The current state of ophthalmic science is such that production thereof cannot be achieved with conventional techniques. In this regard, a normal or transformed corneal endothelial cell marker was selected, which can be used for objectives such as determination of purity of cultured cells, enhancement of purity by sorting, sorting and selection of a normal corneal endothelial cell when inducing a corneal endothelial cell from iPS cells and ES cells.

(Preparation Example: Production of immortalized strain of normal corneal endothelial cells and transformed corneal endothelial cells)

In the present example, immortalized strains of normal corneal endothelial cells and transformed corneal endothelial cells were produced.

### (Culture Method)

Corneal endothelial cells were mechanically peeled off with a basal membrane from a corneal for research purchased from the Seattle Eye Bank. After collagenase was used to detach and collect the corneal endothelial cell from the basal membrane, the cells were subjected to primary culture. As a medium for human cells, Opti-MEM I Reduced-Serum Medium, Liquid (INVITROGEN catalog No.: 31985-070) to which 8% fetal bovine serum (FBS) (BIOWEST, catalog No.: S1820-500), 200 mg/ml CaCl₂·2H₂O (SIGMA catalog No.: C7902-500G), 0.08% chondroitin sulfate (SIGMA catalog No.: C9819-5G), 20 μg/ml ascorbic acid (SIGMA catalog No.: A4544-25G), 50 μg/ml gentamicin (INVITROGEN catalog No.: 15710-064) and 5 ng/ml EGF (INVITROGEN catalog No.: PHG0311) were added and acclimated for a 3T3 feeder cell were used. Further, the cells were cultured a in basal medium to which SB431524 (4-[4-(1,3-benzodioxole-5-yl)-5-(2-pyridinyl)-1H-imidazole-2-yl]benzamide) (1 μmol/1) and SB203580 (4-(4-fluorophenyl)-2-(4-methylsulfonylphenyl)-5 (4-pyridyl)imidazole<4-[4-(4-fluorphenyl)-2-(4-methylsulfinylphenyl)-1H-imidazole-5-yl] pyridine) (1 μmol/l) were added (referred to as “SB203580+SB431542+3T3 acclimated medium”). SB431542 was obtained from TOCRIS (catalog number: 1614). SB203580 was obtained from CALBIOCHEM (catalog number: 559389).

### (Method of Acquisition)

Immortalized cell lines were prepared by introducing an SV40 large T antigen and hTERT gene into each of normal form cells and transformed cells among cultured corneal endothelial cells from a human donor cornea shown in FIG. 1. Specifically, SV40 large T antigen and hTERT gene were amplified by PCR and introduced into a lentiviral vector (pLenti6.3 V5-TOPO; Life Technologies Inc). The lentiviral vector was then used to infect 293T cells (RCB2202; Riken Bioresource Center, Ibaraki, Japan) by a transfection reagent (Fugene H D; Promega Corp., Madison, WI), together with three types of helper plasmids (pLP1, pLP2, pLP/VSVG; Life Technologies Inc.). Culture supernatant comprising viruses was collected after 48 hours from the infection. 5 μg/ml polybrene was used and added to culture solutions of each of cultured normal form corneal endothelial cells and transformed corneal endothelial cells. SV40 large T antigen and hTERT gene were introduce to immortalize the cells.

### Example 1: Flow Cytometry in Transformed Corneal Endothelial Cell and Normal Corneal Endothelial Cell

The inventors screened surface markers of normal human corneal endothelial cells and transformed cells by high-throughput analysis using flow cytometry to proceed with selection of candidate markers.

Human corneal endothelial cells were cultured by the method described above in (Experimental materials and methods). Cells with normal cellular form and transformed cells were subjected to high-throughput analysis by flow cytometry using Human Cell Surface Marker Screening Panel (BD Lyoplate™, BD Bioscience). Expression of surface antigens in each of normal cells and transformed cells was compared, and markers with enhanced expression in normal cells were used as marker candidates for normal cells (Table 2). Meanwhile, markers with enhanced expression in transformed cells were similarly used as markers of transformed cells (Table 3).

(Table 2 Surface Antigens with Enhanced Expression in Normal and Transformed Human Corneal Endothelial Cells)

TABLE 2

| | Human corneal | Monkey corneal |
|---|---|---|
| | endothelial cell | endothelial cell |
| | (normal/transformed) | (normal/transformed) |
| CD166 | 10.09 | 11.80 |
| HLA-A2 | 6.23 | 0.97 |
| CD66 (a, c, d, e) | 3.42 | 0.85 |
| CD98 | 3.22 | 2.94 |
| CD59 | 2.97 | 0.90 |
| CD54 | 2.95 | 0.90 |
| CD340 | 2.82 | 2.31 |
| CD47 | 1.88 | 0.48 |
| EGF-R | 1.74 | 6.58 |
| CD29 | 1.46 | 0.26 |
| CD74 | 1.43 | 2.31 |
| CD165 | 1.26 | 16.13 |
| CD221 | 1.19 | 1.87 |
| CD49a | 1.13 | 3.69 |
| SSEA-4 | 1.03 | 0.30 |
| CD130 | 1.02 | 2.26 |
| CD49f | 1.02 | 2.05 |

(Table 3 Surface Antigens with Enhanced Expression in Transformed Human Corneal Endothelial Cells)

TABLE 3

| | Human corneal | Monkey corneal |
|---|---|---|
| | endothelial cell | endothelial cell |
| | (transformed/normal) | transformed/normal) |
| CD26 | 17.96 | 1.05 |
| CD9 | 10.93 | 3.85 |
| CD49b | 8.91 | 0.82 |
| CD49e | 8.85 | 2.38 |
| CD13 | 8.44 | 1.29 |
| CD99 | 5.63 | 1.05 |
| CD105 | 4.60 | 0.99 |
| CD63 | 4.55 | 1.81 |
| CD58 | 4.06 | 0.25 |
| CD201 | 3.65 | 1.06 |
| CD56 | 3.45 | 0.06 |
| CD44 | 3.25 | 2.98 |
| CD55 | 3.01 | 1.15 |
| CD71 | 2.98 | 1.19 |
| CD73 | 2.94 | 2.02 |
| CD91 | 2.73 | 0.18 |
| HLA-DQ | 2.50 | 1.33 |
| CD164 | 2.47 | 0.83 |
| CD49d | 2.46 | 1.38 |
| CD49c | 2.42 | 0.07 |
| CD90 | 2.38 | 0.49 |
| MICA/B | 2.24 | 1.07 |
| CD46 | 2.15 | 1.21 |
| CD140b | 2.05 | 1.49 |
| CD146 | 1.90 | 0.02 |
| CD147 | 1.79 | 0.09 |
| CD81 | 1.73 | 0.37 |
| CD151 | 1.72 | 0.36 |
| CD200 | 1.49 | 0.18 |
| CD10 | 1.24 | 7.27 |

Further, analysis similar to that for human corneal endothelial cells was performed for normal and transformed corneal endothelial cells separated from cynomolgus monkey. Candidate markers in human corneal endothelial cells exhibiting similar tendency in monkey corneal endothelial cells are shown in Table 4 as candidate markers with particularly high accuracy. The protocol used was in accordance with the method described above in (Experimental materials and methods).

### (Table 4 Representative Surface Antigen Markers of Corneal Endothelial Cells)

TABLE 4

| Normal | Transformed |
|---|---|
| CD98 | CD9 |
| CD166 | CD49e |
| CD340 | CD44 |
| | CD73 |

As a result thereof, the following normal corneal endothelial cell markers and transformed corneal endothelial cell markers were identified. The markers are shown below.

Markers in normal cells of human corneal endothelial cells are CD166, HLA-A2, CD66 (a, c, d, and e), CD98, CD59, CD54, CD340, CD47, EGF-R, CD29, CD74, CD165, CD221, CD49a, SSEA-4, CD130, and CD49f.

Further, markers in transformed cells are CD26, CD9, CD49b, CD49e, CD13, CD99, CD105, CD63, CD58, CD201, CD56, CD44, CD55, CD71, CD73, CD91, HLA-DQ, CD164, CD49d, CD49c, CD90, MICA/B, CD46, CD140b, CD146, CD147, CD81, CD151, CD200, and CD10.

Markers with especially high accuracy are CD98, CD166, and CD340 for normal cells and CD9, CD49e, CD44, and CD73 for transformed cells.

The inventors selected CD166 and CD73 among the above markers to proceed with further analysis. It is demonstrated that CD166 is highly expressed and CD73 is lowly expressed in normal cells, while CD166 is lowly expressed and CD73 is highly expressed in transformed cells.

These surface antigen markers are explained. CD166 is a type I transmembrane protein broadly distributed in cell populations, which are associated with growth and migration. CD166 is expressed in various cells such as hemopoietic cells, endothelial cells, bone marrow mesenchymal stem cells, fibroblasts and the like. CD73 is a membrane-binding enzyme having action to convert AMP to adenosine under a neutral condition, which is expressed in T and B lymphocyte subsets, follicular dendritic cells, endothelial cells, bone marrow stroma cells and the like.

### Example 2: Analysis in Cultured Monkey Corneal Endothelial Cells

In the present Example, cultured monkey corneal endothelial cells were used to analyze cell surface markers thereof in transformed cells and normal cells.

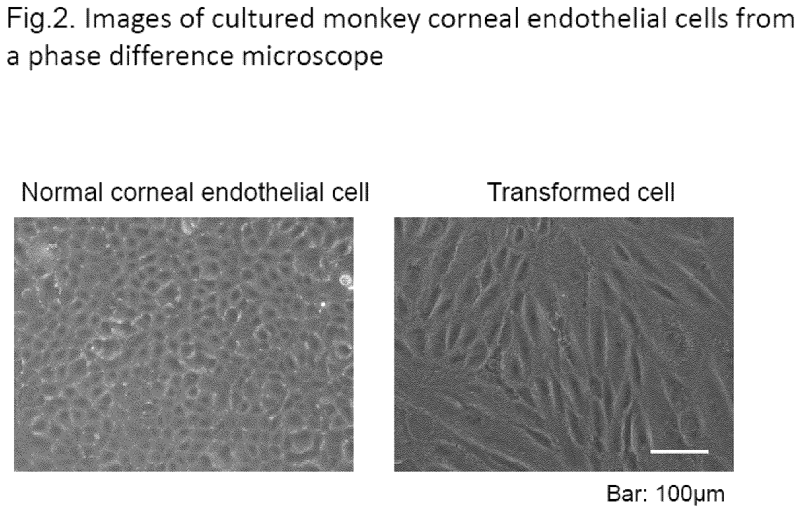

Monkey corneal endothelial cells were cultured. Specifically, a Descemet's membrane including an endothelial cell layer was peeled off from a monkey corneal tissue, and placed in 2 mg/ml Collagenase A (catalog No.: 70164923; Roche Applied Science, Penzberg, Germany) dissolved in DMEM (Gibco-Invitrogen) and incubated at 37° C. After 12 hours, the sample was centrifuged at 1000 rpm for 5 minutes to remove the supernatant. A culture medium was added to a precipitated corneal endothelial cell mass for admixing, and the entire amount was seeded on a 12-well plate coated with FNC Coating Mix (catalog No.: 0407; Athena Enzyme Systems, Baltimore, MD, USA). As a culture medium, DMEM (catalog No.: 12320; Gibco-Invitrogen), to which 10% FBS, 50 μg/ml Gentamicin (catalog number: 15710-064; Invitrogen), and 2 ng/ml basic fibroblast growth factor (catalog No.: 13256-029; bFGF; Invitrogen) were added, was used. Corneal endothelial cells that were able to be cultured in a normal form when cultured with the same method were used as normal corneal endothelial cells and cells undergoing transformation into a fibroblast-like form when cultured with the same method were used as transformed cells in the following study. The normal corneal endothelial cells shown on the left side in the image from a phase difference microscope in FIG. 2 are shown to have a small size for each cell and consist of polygonal, mainly hexagonal, cells. In contrast, cells that have undergone transformation into fibroblast-like form shown on the right side have a large size for each cell and have an elongated shape.

Flow cytometry of CD166 and CD73 in cultured monkey corneal endothelial cells in these two states (normal corneal endothelial cells, transformed cells) was performed. Immunostaining protocol was in accordance with the protocol described in (Experimental materials and methods).

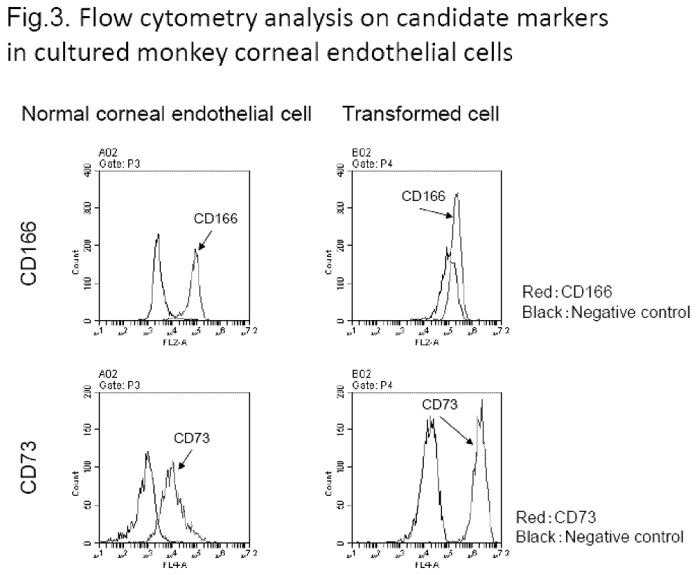

The results are shown in FIG. 3. The left side shows normal corneal endothelial cells and the right side shows transformed cells. The top row shows results from flow cytometry using CD166, while the bottom row shows results using CD73. In view of the results, CD166 is highly expressed in normal corneal endothelial cells compared to the control, while the amount of expression was similar to that for the control in transformed cells. Further, expression of CD73 was found to be strongly enhanced in transformed cells than in normal cells in comparison with the control. This demonstrates that CD166 is highly expressed in normal corneal endothelial cells and CD73 is highly expressed in transformed cells. The result is consistent with results from screening analysis using flow cytometry.

Subsequently, RT-PCR was performed for CD166 and CD73 in cultured monkey corneal endothelial cells in these two states (normal corneal endothelial cells, cells that have undergone transformation into fibroblast-like form). The protocol was in accordance with the protocol described in (Experimental materials and methods).

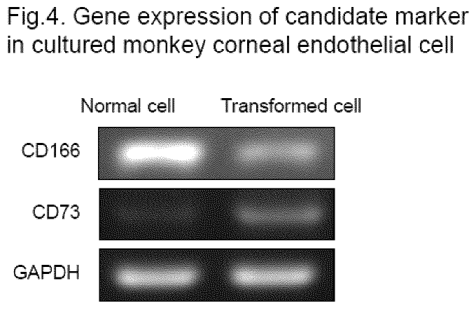

The results are shown in FIG. 4. The bands are for, from the top, CD166, CD73, and GAPDH as a control. The left column is for normal cells and the right column is for transformed cells. It was clearly confirmed from the results that CD166 is highly expressed in normal cells and CD73 is highly expressed in transformed cells also at gene level. This is consistent with the results from screening analysis by flow cytometry.

Subsequently, expression of CD166 and CD73 in cultured monkey corneal endothelial cells in these two states (normal corneal endothelial cells and transformed cells) was also studied with immunostaining. The protocol was in accordance with the protocol described in (Experimental materials and methods).

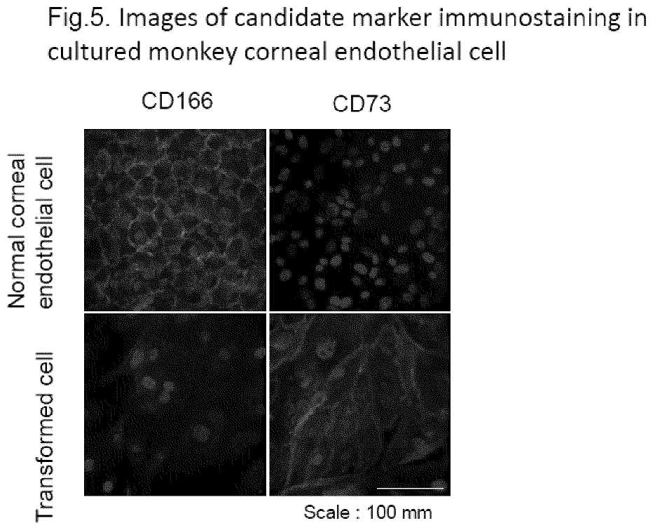

The results are shown in FIG. 5. The results are shown, from the left side, for CD166 and CD73. The top row shows normal cells and the bottom row shows transformed cells. The results show that CD166 is highly expressed and CD73 is lowly expressed in normal cells, while, CD166 is lowly expressed and CD73 is highly expressed in transformed cells. It was possible to confirm that the results are consistent with the results of screening analysis from flow cytometry. When detailed studied were conducted with CD166 and CD73 as representative normal cell marker and a transformed cell marker, respectively, in view of the above results, they were demonstrated to be usable as a marker in flow cytometry, PCR, and immunostaining.

### Example 3: Analysis in Cultured Monkey Corneal Endothelial Cells

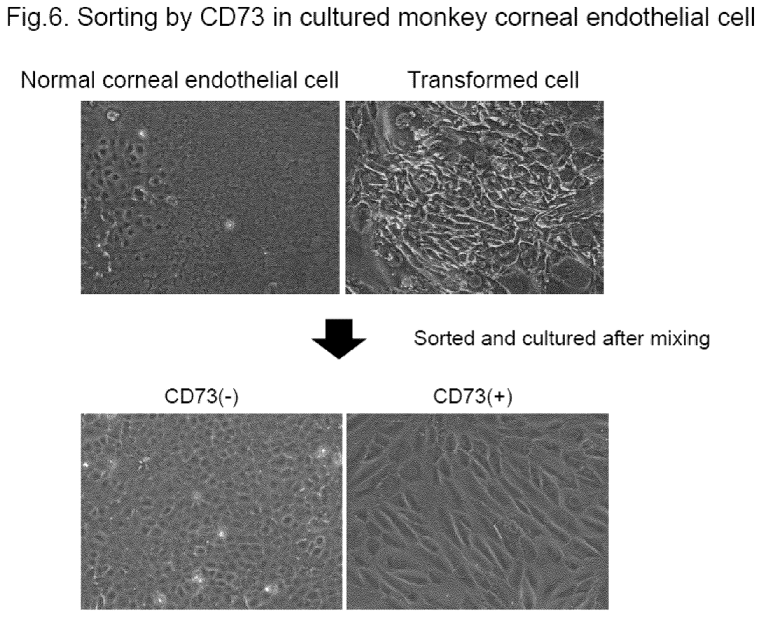

The present Example sorted normal and transformed monkey cells with CD73 as an illustration and demonstrated that it is applicable to cell purification. The top left portion of FIG. 6 shows an image of normal monkey corneal endothelial cells and the top right shows an image of transformed cells from a phase difference microscope. After mixing monkey corneal endothelial cells in these two states (normal corneal endothelial cells and transformed cells) at a ratio of 1:1, flow cytometry analysis was performed with CD73. Cells were sorted and cultured in two separate groups, high expression cell group and low expression cell group of CD73. The bottom left is a picture from a phase difference microscope after sorting of CD73 low expression cells (CD73(−)) and the bottom right is of CD73 high expression cells (CD73(+)). CD73(−) is a cell with a polygonal form which the form of normal cells, while CD73(+) is a cell with a transformed fibroblast-like form. This demonstrates that CD73 can be used as a marker for sorting normal cells and transformed fibroblast-like cells.

### Example 4: Analysis in Immortalized Human Corneal Endothelial Cells

The present Example used immortalized human corneal endothelial cells prepared in the Preparation Example to analyze cell surface markers in transformed cells and normal cells.

A Descemet's membrane including an endothelial cell layer was peeled off from a human corneal tissue, and placed in 2 mg/ml Collagenase A (catalog No.: 70164923; Roche Applied Science, Penzberg, Germany) dissolved in OPTIMEM-I and incubated at 37° C. After 12 hours, the sample was centrifuged at 1000 rpm for 5 minutes to remove the supernatant. A culture medium was then added to a precipitated corneal endothelial cell mass for admixing, and the entire amount was seeded on a 12-well plate coated with FNC Coating Mix (catalog No.: 0407; Athena Enzyme Systems, Baltimore, MD, USA). Cultured human corneal endothelial cells were introduced with SV40 and hTERT gene to produce immortalized cells. Each of normal cell forms and fibroblast-like transformed cells was cloned from the produced immortalized human corneal endothelial cells and the clones were used in the following study as normal cells and transformed cells.

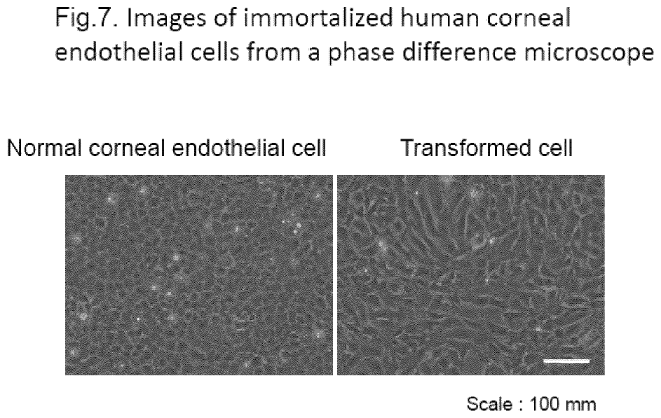

FIG. 7 shows images of immortalized human corneal endothelial cells from a phase difference microscope. The left side shows normal corneal endothelial cells and the right side shows transformed cells. As in cultured monkey cells, normal corneal endothelial cells shown on the left side are shown to have a small size for each cell and consist of polygonal, mainly hexagonal, cells. In contrast, cells that have undergone transformation into fibroblast-like form shown on the right side have a large size for each cell and have an elongated shape.

With CD73 as an example, a test was carried out to see whether a marker in the present invention can be used to sort and purify normal cells. Mixed culture cells consisting of normal form and transformed immortalized human corneal endothelial cells were analyzed by flow cytometry with CD73.

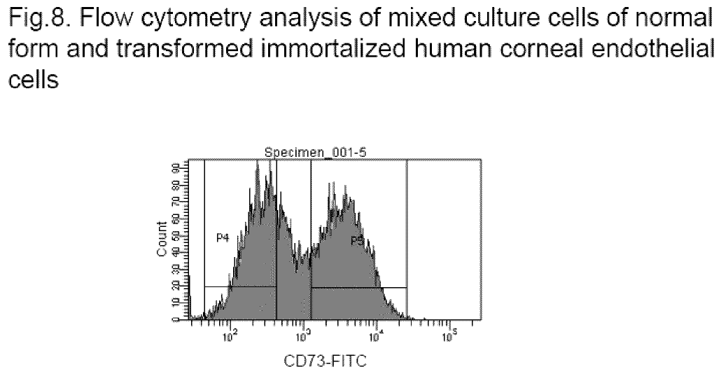

The results are shown in FIG. 8. It can be understood from flow cytometry with CD73 that the cells consist of two groups, high expression cell group and low expression cell group of CD73. Subsequently, CD73 low expression cells indicated by P4 and CD73 high expression cells indicated by P5 in FIG. 8 were sorted and cultured.

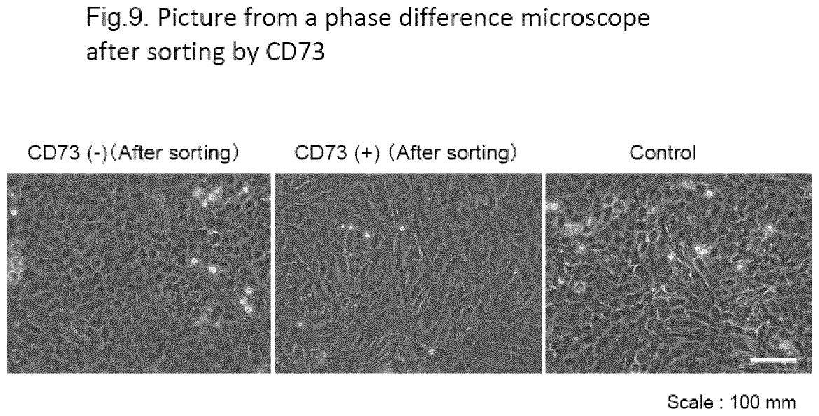

The results are shown in FIG. 9. The left side is a picture from a phase difference microscope after sorting of CD73 low expression cells (CD73(−)) and the middle portion is for CD73 high expression cells (CD73(+)). CD73(−) is a cell with a polygonal form which is the form of normal cells, while CD73(+) is a transformed cell with a fibroblast-like form. The right side is an image from a phase difference microscope of cells cultured without sorting as a control, where cells with a normal form and transformed cells co-exist. In view of the above, it is demonstrated that CD73 can be used as a marker for sorting normal cells and transformed fibroblast-like cells.

Expression was subsequently analyzed for a candidate marker after sorting with CD73. In this analysis, expression of CD73 and CD166 was analyzed. The protocol was in accordance with the protocol described in (Experimental materials and methods).

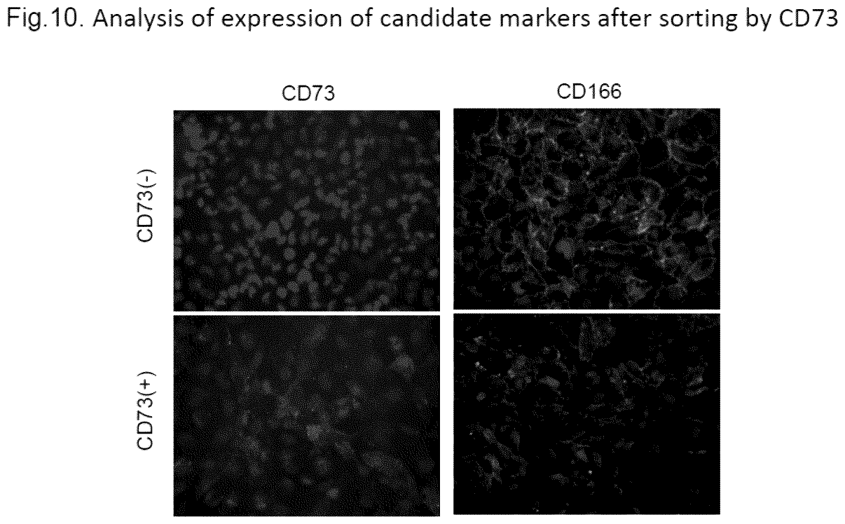

The results are shown in FIG. 10. The top row shows cells sorted as CD73(−) and the bottom row shows cells sorted as CD73(+). The left side shows staining by CD73 and the right side shows staining with CD166. CD73 was not stained in CD73(−), but CD166 was intensely stained. Further, CD73 was stained in CD73(+), while expression of CD166 was found to be attenuated therein in comparison to CD73(−).

Expression was subsequently analyzed for a function associated marker after sorting with CD73. In this analysis, expression of ZO-1 and Na⁺/K⁺-ATPase was analyzed. The protocol was in accordance with the protocol described in (Experimental materials and methods).

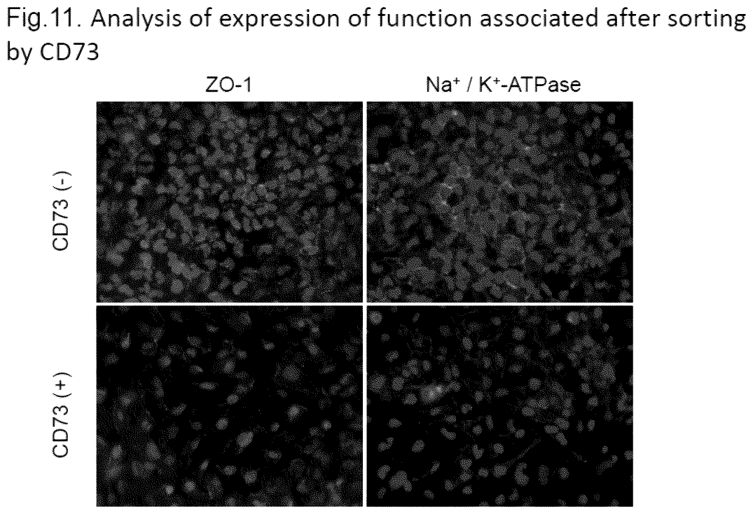

The results are shown in FIG. 11. The top row shows CD73(−) and the bottom row shows CD73(+). The left side shows expression of ZO-1 and the right side shows expression of Na⁺/K⁺-ATPase. It is shown that both ZO-1 and Na⁺/K⁺-ATPase indicating normal function are expressed significantly more in CD73(−). Thus, it is demonstrated that CD73(−) is a marker that can also distinguish normal cells from transformed cells with respect to functional aspects.

### Example 5: Method of Preparing Sample with Concentrated Normal Cells Using a Marker of the Present Invention

Cultured corneal endothelial cells can be sorted by using a marker for a transformed cell of the present invention to remove transformed cells for purified corneal endothelial cells with a normal form and normal function. Further, corneal endothelial cells with a normal form and normal function can be selectively cultured by sorting cells with a normal cell marker of the present invention. Further, when a sample is contaminated with cells other than corneal endothelial cells such as corneal epithelial cells, corneal stromal cells, conjunctival epithelial cells, or conjunctival stromal cells when culturing corneal endothelial cells from a corneal tissue, the corneal endothelial cells can be purified by sorting with a marker of the present invention. Further, by using this technique, corneal endothelial cells can be purified by sorting with a marker of the present invention from a cell group induced from iPS cells or ES cells.

### Example 6: Therapy Using Sample with Concentrated Normal Cells Using a Marker of the Present Invention

Therapy can be administered by transplanting cultured corneal endothelial cells or corneal endothelial cells induced from iPS cells or ES cells with enhanced purity produced in Example 5 as a corneal endothelial cell sheet using or without using a carrier into a corneal endothelial dysfunction patient. Further, therapy can be administered by infusion or injection of the cells into the eye, such as within the anterior chamber, as a cell suspension.

As described above, the present invention has been exemplified using preferable embodiments of the present invention. However, it is understood that the scope of the present invention should be construed only by the scope of claims. The present application claims priority to Japanese Patent Application No. 2013-157597, whose entire content is incorporated herein by reference. It is understood that patents, patent applications and literatures cited herein are incorporated herein by reference, as if the contents thereof are specifically described herein.

### INDUSTRIAL APPLICABILITY

A marker for distinguishing normal from cells fibroblast-like transformed cells for ocular cells is provided. In addition, a technique available in industries involved in techniques associated with corneal transplantation (cell culture industry, pharmaceuticals or the like) is provided. [Sequence Listing Free Text]
SEQ ID NO: 1: Nucleic acid sequence of heavy subunit protein of CD98 (Gene ID (Entrez): 6520; NM_001013251 (NM_001012661) and NP_001012680)
SEQ ID NO: 2: Amino acid sequence of heavy subunit protein of CD98 (Gene ID (Entrez): 6520; NM_001013251 (NM_001012661) and NP_001012680)
SEQ ID NO: 3: Nucleic acid sequence of light subunit protein of CD98 (Gene ID (Entrez): 8140 NM_003486; NP_003477)
SEQ ID NO: 4: Amino acid sequence of light subunit protein of CD98 (Gene ID (Entrez): 8140 NM_003486; NP_003477)
SEQ ID NO: 5: Nucleic acid sequence of CD166 (Gene ID (Entrez): 214 (human), NM_001243280 (mRNA), NP_001230209 (protein))
SEQ ID NO: 6: Amino acid sequence of CD166 (Gene ID (Entrez): 214 (human), NM_001243280 (mRNA), NP_001230209 (protein))
SEQ ID NO: 7: Nucleic acid sequence of CD340 (Entrez Gene ID: (Hu) 2064, and NM_0010005862.1)
SEQ ID NO: 8: Amino acid sequence of CD340 (Entrez Gene ID: (Hu) 2064, NM_0010005862.1)
SEQ ID NO: 9: Nucleic acid sequence of CD9 (Entrez Gene ID: 928, NM_001769 (Hu), NP_001760 (Hu))
SEQ ID NO: 10: Amino acid sequence of CD9 (Entrez Gene ID: 928, NM_001769 (Hu), NP_001760 (Hu))
SEQ ID NO: 11: Nucleic acid sequence of CD49e (Entrez Gene ID: 3678, NM_002205 (Hu), NP_002196 (Hu))
SEQ ID NO: 12: Amino acid sequence of CD49e (Entrez Gene ID: 3678, NM_002205 (Hu), NP_002196 (Hu))
SEQ ID NO: 13: Nucleic acid sequence of CD44 (Entrez Gene ID: 960, NM_000610, NP_000601)
SEQ ID NO: 14: Amino acid sequence of CD44 (Entrez Gene ID: 960, NM_000610, NP_000601)
SEQ ID NO: 15: Nucleic acid sequence of CD73 (Entrez Gene ID: 4907, NM_001204813, NP_001191742)
SEQ ID NO: 16: Amino acid sequence of CD73 (Entrez Gene ID: 4907, NM_001204813, NP_001191742)

SEQ ID NO: 17: primer CD166-F
CCCCAGAGGAATTTTTGTTTTAC
SEQ ID NO: 18: primer CD166-R
AGCCTGATGTTATCTTTCATCCA
SEQ ID NO: 19: primer CD73-F
GTTCCTGTAGTCCAGGCCTATG
SEQ ID NO: 20: primer CD73-R
ACATTTCATCCGTGTGTCTCAG
SEQ ID NO: 21: primer GAPDH-F
GAGTCAACGGATTTGGTCGT
SEQ ID NO: 22: primer GAPDH-R
TTGATTTTGGAGGGATCTCG

1-25. (canceled)

26. A method for determining the fibroblastic status of a corneal endothelial cell, comprising:
(a) contacting a sample comprising the corneal endothelial cell with one or more detection agents selected from:
(i) one or more detection agents selective for a cell-surface marker of corneal endothelial cells with a non-fibroblastic phenotype selected from the group consisting of CD166, HLA-A2, CD66a, CD66c, CD66d, CD66e, CD98, CD59, CD54, CD340, CD47, EGF-R, CD29, CD74, CD165, CD221, CD49a, SSEA-4, CD130, and CD49f, and
(ii) one or more detection agents selective for a cell-surface marker of corneal endothelial cells with a fibroblastic phenotype selected from the group consisting of CD26, CD9, CD49b, CD49e, CD13, CD99, CD105, CD63, CD58, CD201, CD56, CD44, CD55, CD71, CD73, CD91, HLA-DQ, CD164, CD49d, CD49c, CD90, MICA/B, CD46, CD140b, CD146, CD147, CD81, CD151, CD200, and CD10;
(b) detecting any recognition of the marker(s) by the detection agent(s); and
(c) determining the cell has a non-fibroblastic phenotype when a detected cell-surface marker of corneal endothelial cells with a non-fibroblastic phenotype is expressed at a higher level as compared to its expression level by a corneal endothelial cell with a fibroblastic phenotype, or
determining the cell has a fibroblastic phenotype when a detected cell-surface marker of corneal endothelial cells with a fibroblastic phenotype is expressed at a higher level as compared to its expression level by a corneal endothelial cell with a non-fibroblastic phenotype.

27. The method of claim 26, wherein the method comprises contacting the sample with one or more detection agent(s) selective for a cell-surface marker selected from CD166, CD98, and CD340.

28. The method of claim 26, wherein the method comprises contacting the sample with one or more detection agent(s) selective for a cell-surface marker selected from CD9, CD49e, CD44, and CD73.

29. The method of claim 26, wherein the method comprises contacting the sample with a detection agent selective for cell-surface marker CD166.

30. The method of claim 26, wherein the method comprises contacting the sample with a detection agent selective for cell-surface marker CD73.

31. The method of claim 26, wherein the method comprises contacting the sample with a detection agent selective for cell-surface marker CD166 and a detection agent selective for cell-surface marker CD73.

32. A method for differentiating between corneal endothelial cells with a non-fibroblastic phenotype and corneal endothelial cells with a fibroblastic phenotype in a sample comprising corneal endothelial cells with a non-fibroblastic phenotype and corneal endothelial cells with a fibroblastic phenotype, comprising at least one of:
detecting corneal endothelial cells with a non-fibroblastic phenotype in the sample by detecting at least one cell-surface marker of corneal endothelial cells with a non-fibroblastic phenotype selected from the group consisting of CD166, HLA-A2, CD66a, CD66c, CD66d, CD66e, CD98, CD59, CD54, CD340, CD47, EGF-R, CD29, CD74, CD165, CD221, CD49a, SSEA-4, CD130, and CD49f, wherein the detected cell-surface marker of corneal endothelial cells with a non-fibroblastic phenotype is expressed at a higher level as compared to the expression level of the same cell-surface marker by a corneal endothelial cell with a fibroblastic phenotype, and
detecting corneal endothelial cells with a fibroblastic phenotype in the sample by detecting at least one cell-surface marker of corneal endothelial cells with a fibroblastic phenotype selected from the group consisting of CD26, CD9, CD49b, CD49e, CD13, CD99, CD105, CD63, CD58, CD201, CD56, CD44, CD55, CD71, CD73, CD91, HLA-DQ, CD164, CD49d, CD49c, CD90, MICA/B, CD46, CD140b, CD146, CD147, CD81, CD151, CD200, and CD10, wherein the detected cell-surface marker of corneal endothelial cells with a fibroblastic phenotype is expressed at a higher level as compared to the expression level of the same cell-surface marker by a corneal endothelial cell with a non-fibroblastic phenotype.

33. The method of claim 32, wherein the method comprises detecting one or more cell-surface markers selected from CD166, CD98, and CD340.

34. The method of claim 32, wherein the method comprises detecting one or more cell-surface markers selected from CD9, CD49e, CD44, and CD73.

35. The method of claim 32, wherein the method comprises detecting cell-surface marker CD166.

36. The method of claim 32, wherein the method comprises detecting cell-surface marker CD73.

37. The method of claim 32, wherein the method comprises detecting cell-surface marker CD166 and cell-surface marker CD73.

38. A method of preparing a corneal endothelial cell sample with an elevated ratio of corneal endothelial cells with a non-fibroblastic phenotype to corneal endothelial cells with a fibroblastic phenotype, comprising:
in a corneal endothelial cell sample comprising cells with a non-fibroblastic phenotype and corneal endothelial cells with a fibroblastic phenotype, elevating the relative amount of corneal endothelial cells with a non-fibroblastic phenotype expressing one or more cell-surface markers of corneal endothelial cells with a non-fibroblastic phenotype selected from the group consisting of CD166, HLA-A2, CD66a, CD66c, CD66d, CD66e, CD98, CD59, CD54, CD340, CD47, EGF-R, CD29, CD74, CD165, CD221, CD49a, SSEA-4, CD130, and CD49f, at a higher level as compared to its expression level by a corneal endothelial cell with a fibroblastic phenotype, thereby obtaining a corneal endothelial cell sample with an elevated ratio of corneal endothelial cells with a non-fibroblastic phenotype to corneal endothelial cells with a fibroblastic phenotype.

39. The method of claim 38, further comprising, administering the corneal endothelial cell sample with an elevated ratio of corneal endothelial cells with a non-fibroblastic phenotype to corneal endothelial cells with a fibroblastic phenotype to a subject in need of prevention or treatment of a corneal endothelial disease.

40. The method of claim 38, wherein the method comprises elevating the relative amount of corneal endothelial cells expressing one or more cell-surface markers of corneal endothelial cells with a non-fibroblastic phenotype selected from CD166, CD98, and CD340 at a higher level as compared to its expression level by a corneal endothelial cell with a fibroblastic phenotype.

41. The method of claim 38, wherein the method comprises elevating the relative amount of corneal endothelial cells expressing cell-surface marker CD166 at a higher level as compared to its expression level by a corneal endothelial cell with a fibroblastic phenotype.
